(19) United States
(12) Patent Application Publication
(10) Pub. No.: US 2017/0135990 A1
(43) Pub. Date: May 18, 2017

(54) PHARMACEUTICAL COMPOSITIONS COMPRISING A 5,5-FUSED HETEROARYLENE FLAVIVIRIDAE INHIBITOR AND THEIR USE FOR TREATING OR PREVENTING FLAVIVIRIDAE INFECTION

(71) Applicant: IDENIX PHARMACEUTICALS LLC, Cambridge, MA (US)

(72) Inventors: Adel M. Moussa, Burlington, MA (US); Benjamin Alexander Mayes, Boston, MA (US); Sindhura Ganga, Waltham, MA (US)

(73) Assignee: Idenix Pharmaceuticals LLC, Cambridge, MA (US)

(21) Appl. No.: 15/122,954

(22) PCT Filed: Mar. 4, 2015
(86) PCT No.: PCT/US2015/018572

§ 371 Date: Sep. 1, 2016

Related U.S. Application Data
(60) Provisional application No. 61/948,466, filed on Mar. 5, 2014.

(51) Int. Cl. A61K 31/4184; A61K 45/06; A61K 9/16

(52) U.S. Cl. CPC A61K 31/4184; A61K 9/16; A61K 9/1635; A61K 9/1641; A61K 45/06

(57) ABSTRACT

Provided herein are spray-dried particles comprising a 5,5-fused heteroarylene hepatitis C virus inhibitor compound and methods of their preparation. Also provided herein are pharmaceutical compositions comprising a 5,5-fused heteroarylene hepatitis C virus inhibitor compound, method of their preparation, and their use for treating or preventing hepatitis C virus infection.

### FIELD

Provided herein are spray-dried particles comprising a 5,5-fused heteroarylene Flaviviridae inhibitor compound and methods of their preparation. Also provided herein are pharmaceutical compositions comprising a 5,5-fused heteroarylene Flaviviridae inhibitor compound, method of their preparation, and their use for treating or preventing Flaviviridae infection.

### BACKGROUND

The Flaviviridae family of viruses comprises at least three distinct genera: pestiviruses, which cause disease in cattle and pigs; flaviviruses, which are the primary cause of diseases such as West Nile virus, dengue fever, and yellow fever; and hepaciviruses, which sole member is hepatitis C virus (HCV). The flavivirus genus includes more than 68 members separated into groups on the basis of serological relatedness. Calisher et al., J Gen. Virol. 1993, 70, 37-43. Clinical symptoms vary and include fever, encephalitis, and hemorrhagic fever. Fields Virology, Editors: Fields et al., Lippincott-Raven Publishers, Philadelphia, Pa., 1996, Chapter 31, 931-959. Flaviviruses of global concern that are associated with human disease include the dengue hemorrhagic fever viruses (DHF), yellow fever virus, shock syndrome, and Japanese encephalitis virus. Halstead, Rev. Infect. Dis. 1984, 6, 251-264; Halstead, Science 1988, 239, 476-481; Monath, New Eng. J Med. 1988, 319, 641-643.

Hepatitis C virus (HCV) infection is a major global public health problem. The global prevalence of HCV infection is estimated to be about 3%, resulting in approximately 170 million HCV-infected people worldwide. Most of those infected develop persistent, chronic infection. An estimated 20-50% of the people with chronic HCV infection are at risk of developing long-term complications such as cirrhosis and hepatocellular carcinoma. HCV infection is one of the main causes of liver cirrhosis and hepatocellular carcinoma, and every year more than 350,000 people die from HCV-related liver diseases. World Health Organization 2012, Hepatitis C Fact Sheet No. 164; July 2012; World Health Organization 2003, Global Alert and Response, Hepatitis C.

HCV is an enveloped virus containing a positive-sense single-stranded RNA genome of approximately 9.4 kb. Kato et al., Proc. Natl. Acad. Sci. USA 1990, 87, 9524-9528; Kato, Acta Medica Okayama, 2001, 55, 133-159. The viral genome consists of a 5′ untranslated region (UTR), a long open reading frame encoding a polyprotein precursor of approximately 3011 amino acids, and a short 3′ UTR. The 5′ UTR is the most highly conserved part of the HCV genome and is important for the initiation and control of polyprotein translation. Translation of the HCV genome is initiated by a cap-independent mechanism known as an internal ribosome entry. This mechanism involves the binding of ribosomes to an RNA sequence known as the internal ribosome entry site (IRES). An RNA pseudoknot structure has recently been determined to be an essential structural element of the HCV IRES. Viral structural proteins include a nucleocapsid core protein (C) and two envelope glycoproteins E1 and E2. HCV also encodes two proteinases, a zinc-dependent metalloproteinase encoded by the NS2-NS3 region and a serine proteinase encoded in the NS3 region. These proteinases are required for cleavage of specific regions of the precursor polyprotein into mature peptides. The carboxyl half of nonstructural protein 5, NS5B, contains the RNA-dependent RNA polymerase. The function of the remaining nonstructural proteins, NS4A, NS4B, and NS5A (the amino-terminal half of nonstructural protein 5) remain unknown.

The current standard treatment for the infection of hepatitis C virus, a member of the Flaviviridae family, includes a combination of a protease inhibitor, boceprevir or telaprevir, with pegylated interferon and ribavirin. There is an unmet need for antivirals that are effective and well tolerated for treating a Flaviviridae infection.

### SUMMARY OF THE DISCLOSURE

Provided herein are spray-dried particles comprising a compound of [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester, having the structure of Formula A1;

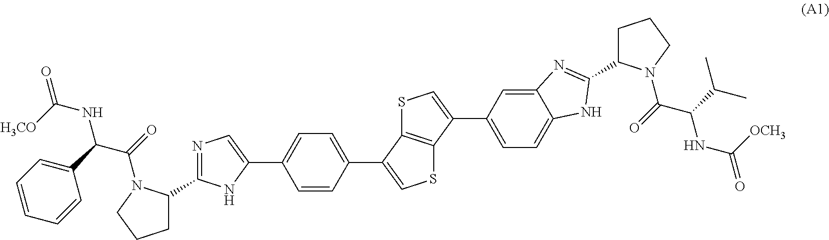

or [(S)-1-((S)-2-{5-[4-(6-{(S)-2-[1-((R)-2-methoxycarbonylamino-2-phenylacetyl)-pyrrolidin-2-yl]-3H-benzoimidazol-5-yl}-thieno[3,2-b]-thiophen-3-yl)-phenyl]-1H-imidazol-2-yl}pyrrolidine-1-carbonyl)-2-methylpropyl]-carbamic acid methyl ester, having the structure of Formula A2;

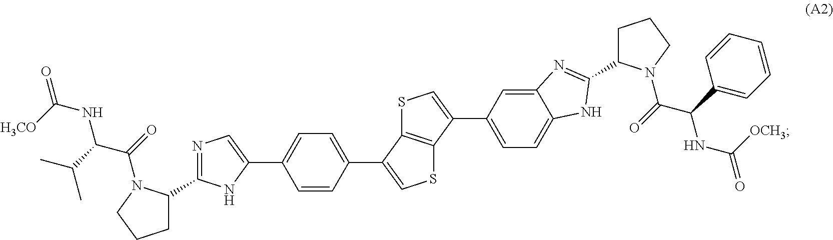

or an isotopic variant thereof, or a pharmaceutically acceptable salt thereof, or a pharmaceutically acceptable solvate thereof; and a pharmaceutically acceptable excipient.

Also provided herein is a granular pharmaceutical composition comprising (i) spray-dried particles comprising a compound of Formula A1 or A2, or an isotopic variant thereof, or a pharmaceutically acceptable salt thereof, or a pharmaceutically acceptable solvate thereof; and a first pharmaceutically acceptable excipient; and (ii) a second pharmaceutically acceptable excipient.

Additionally provided herein is a granular composition comprising (i) spray-dried particles comprising a compound of Formula A1 or A2, or an isotopic variant thereof, or a pharmaceutically acceptable salt thereof, or a pharmaceutically acceptable solvate thereof; and a dispersant or carrier; and (ii) a glidant and a lubricant.

Provided herein is a pharmaceutical composition comprising (i) spray-dried particles comprising a compound of Formula A1 or A2, or an isotopic variant thereof, or a pharmaceutically acceptable salt thereof, or a pharmaceutically acceptable solvate thereof; and a first pharmaceutically acceptable excipient; and (ii) a second pharmaceutically acceptable excipient. In one embodiment, the pharmaceutical composition is solid. In another embodiment, the pharmaceutical composition is a tablet. In yet another embodiment, the pharmaceutical composition is a capsule.

Provided herein is a pharmaceutical composition comprising (i) spray-dried particles comprising a compound of Formula A1 or A2, or an isotopic variant thereof, or a pharmaceutically acceptable salt thereof, or a pharmaceutically acceptable solvate thereof; and a dispersant or carrier; and (ii) a disintegrant, a filler, a glidant, and a lubricant. In one embodiment, the pharmaceutical composition is solid. In another embodiment, the pharmaceutical composition is a tablet. In yet another embodiment, the pharmaceutical composition is a capsule.

Provided herein is a pharmaceutical composition comprising (i) an intragranular components comprising: spray-dried particles comprising a compound of Formula A1 or A2, or an isotopic variant thereof, or a pharmaceutically acceptable salt thereof, or a pharmaceutically acceptable solvate thereof; and an intragranular excipient; and (ii) extragranular components comprising: an extragranular excipient. In one embodiment, the pharmaceutical composition is a tablet. In another embodiment, the pharmaceutical composition is a capsule.

Provided herein is a pharmaceutical composition comprising (i) an intragranular components comprising: spray-dried particles comprising a compound of Formula A1 or A2, or an isotopic variant thereof, or a pharmaceutically acceptable salt thereof, or a pharmaceutically acceptable solvate thereof; and a glidant and a lubricant; and (ii) extragranular components comprising: a disintegrant, a filler, a glidant, and a lubricant. In one embodiment, the pharmaceutical composition is a tablet. In another embodiment, the pharmaceutical composition is a capsule.

Provided herein is a granular pharmaceutical composition comprising a compound of Formula A1 or A2, or an isotopic variant thereof, or a pharmaceutically acceptable salt thereof, or a pharmaceutically acceptable solvate thereof; and a pharmaceutically acceptable excipient.

Provided herein is a granular pharmaceutical composition comprising a compound of Formula A1 or A2, or an isotopic variant thereof, or a pharmaceutically acceptable salt thereof, or a pharmaceutically acceptable solvate thereof; and a dispersant or carrier, a glidant, and a lubricant.

Provided herein is a solid pharmaceutical composition comprising a compound of Formula A1 or A2, or an isotopic variant thereof, or a pharmaceutically acceptable salt thereof, or a pharmaceutically acceptable solvate thereof; and a pharmaceutically acceptable excipient. In one embodiment, the pharmaceutical composition is a tablet. In another embodiment, the pharmaceutical composition is a capsule.

Provided herein is a pharmaceutical composition comprising a compound of Formula A1 or A2, or an isotopic variant thereof, or a pharmaceutically acceptable salt thereof, or a pharmaceutically acceptable solvate thereof; and a dispersant or carrier, a disintegrant, a filler, a glidant, and a lubricant. In one embodiment, the pharmaceutical composition is solid. In another embodiment, the pharmaceutical composition is a tablet. In yet another embodiment, the pharmaceutical composition is a capsule.

Provided herein is a pharmaceutical composition comprising (i) an intragranular components comprising: a compound of Formula A1 or A2, or an isotopic variant thereof, or a pharmaceutically acceptable salt thereof, or a pharmaceutically acceptable solvate thereof; and an intragranular excipient; and (ii) extragranular components comprising: an extragranular excipient. In one embodiment, the pharmaceutical composition is a tablet. In another embodiment, the pharmaceutical composition is a capsule.

Provided herein is a pharmaceutical composition comprising (i) an intragranular components comprising: a compound of Formula A1 or A2, or an isotopic variant thereof, or a pharmaceutically acceptable salt thereof, or a pharmaceutically acceptable solvate thereof; and a dispersant or carrier, a glidant and a lubricant; and (ii) extragranular components comprising: a disintegrant, a filler, a glidant, and a lubricant. In one embodiment, the pharmaceutical composition is a tablet. In another embodiment, the pharmaceutical composition is a capsule.

Provided herein is a method for treating or preventing a Flaviviridae infection, in one embodiment, an HCV infection, in a subject, comprising administering to the subject a therapeutically effective amount of a compound of Formula A1 or A2 in the form of spray-dried particles or a pharmaceutical composition provided herein.

Provided herein is a method for treating, preventing, or ameliorating one or more symptoms of a liver disease or disorder associated with a Flaviviridae infection, in one embodiment, an HCV infection, in a subject, comprising administering to the subject a therapeutically effective amount of a compound of Formula A1 or A2 in the form of spray-dried particles or a pharmaceutical composition provided herein.

Provided herein is a method for inhibiting replication of a Flaviviridae virus, in one embodiment, a hepatitis C virus, in a subject, comprising administering to the subject a therapeutically effective amount of a compound of Formula A1 or A2 in the form of spray-dried particles or a pharmaceutical composition provided herein.

### BRIEF DESCRIPTION OF THE DRAWINGS

FIG. 1 depicts a particle size distribution of an exemplary spray-dried dispersion prepared from a solution comprising 5% by weight of [(S)-1-((S)-2-{6-[6-(4-{(S)-2-[1-((R)-2-methoxycarbonylamino-2-phenyl-acetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methyl-propyl]-carbamic acid methyl ester (compound A1), 15% by weight of povidone PVP-K30, 64% by weight of tetrahydrofuran, and 16% of methanol.

FIG. 2 depicts a SEM image of an exemplary spray-dried dispersion prepared from a solution comprising 5% by weight of compound A1, 15% by weight of povidone PVP-K30, 64% by weight of tetrahydrofuran, and 16% of methanol.

FIG. 3 depicts a particle size distribution of an exemplary spray-dried dispersion prepared from a solution comprising 5% by weight of compound A1, 15% by weight of povidone PVP-K30, 72% by weight of methanol, and 8% by weight of water.

FIG. 4 depicts a SEM image of an exemplary spray-dried dispersion prepared from a solution comprising 5% by weight of compound A1, 15% by weight of povidone PVP-K30, 72% by weight of methanol, and 8% by weight of water.

FIG. 5 depicts a particle-size distribution of exemplary granules prepared from a spray-dried dispersion made from a solution comprising 5% by weight of compound A1, 15% by weight of povidone PVP-K30

### DETAILED DESCRIPTION

To facilitate understanding of the disclosure set forth herein, a number of terms are defined below.

Generally, the nomenclature used herein and the laboratory procedures in biology, biochemistry, organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject, in one embodiment, a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The term “therapeutically effective amount” are meant to include the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician. A therapeutically effective amount of a compound provided herein can be administered in one dose (i.e., a single dose administration) or divided and administered over time (i.e., continuous administration or multiple sub-dose administration). Single dose administration, continuous administration, or multiple sub-dose administration can be repeated, for example, to maintain the level of the compound in a biological molecule (e.g., a protein, enzyme, RNA, or DNA), cell, tissue, system, animal, or human.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 22nd ed.; Loyd et al., Eds.; The Pharmaceutical Press, 2012; Handbook of Pharmaceutical Excipients, 7th ed.; Rowe et al., Eds.; The Pharmaceutical Press, 2012; Handbook of Pharmaceutical Additives, 3rd ed.; Ash and Ash Eds.; Synapse Information Resources, Inc., 2007; Pharmaceutical Preformulation and Formulation, 2nd ed.; Gibson Ed.; CRC Press LLC, 2009.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The term “percent by weight” or “% by weight” refers to the weight of a specified component (e.g., an active compound or excipient) in a composition (e.g., a pharmaceutical composition) as a percentage of the total weight of the composition. Thus, the sum of the weight percentages of all the components in a composition is 100%.

The terms “active ingredient” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient” and “active substance” may be an optically active isomer or an isotopic variant of a compound described herein.

The terms “drug,” “therapeutic agent,” and “chemotherapeutic agent” refer to a compound, or a pharmaceutical composition thereof, which is administered to a subject for treating, preventing, or ameliorating one or more symptoms of a condition, disorder, or disease.

The term “hepatitis C virus” or “HCV” refers to a viral species or a variant thereof, a pathogenic strain of which causes hepatitis C. Examples of HCV include, but are not limited to, HCV genotypes 1, 2, 3, 4, 5, 6, 7, 8, 9, 10, 11, and subtype 1a, 1b, 1c, 2a, 2b, 2c, 3a, 3b, 4a, 4b, 4c, 4d, 4e, 5a, 6a, 7a, 7b, 8a, 8b, 9a, 10a, and 11a. In certain embodiments, an HCV variant is an HCV species that contains a protein substantially homologous to a native HCV protein, i.e., a protein having one or more naturally or non-naturally occurring amino acid deletions, insertions or substitutions (e.g., derivatives, homologs, and fragments), as compared to the amino acid sequence of the native protein. The amino acid sequence of a protein of an HCV variant is at least about 80% identical, at least about 90% identical, or at least about 95% identical to a native HCV protein. In certain embodiments, the HCV variant contains an NS5A protein variant.

The term “NS5A” refers to nonstructural protein 5A or a variant thereof. NS5A variants include proteins substantially homologous to a native NS5A, i.e., proteins having one or more naturally or non-naturally occurring amino acid deletions, insertions or substitutions (e.g., NS5A derivatives, homologs, and fragments), as compared to the amino acid sequence of a native NS5A. The amino acid sequence of an NS5A variant is at least about 80% identical, at least about 90% identical, or at least about 95% identical to a native NS5A.

In certain embodiments, “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, the compound comprises about 95% or more of one enantiomer and about 5% or less of the other enantiomer based on the total weight of the racemate in question.

In describing an optically active compound, the prefixes R and S are used to denote the absolute configuration of the molecule about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the molecule, R and S.

The term “isotopic variant” refers to a compound that contains an unnatural proportion of an isotope at one or more of the atoms that constitute such compounds. In certain embodiments, an “isotopic variant” of a compound contains unnatural proportions of one or more isotopes, including, but not limited to, hydrogen (H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (^{1s}F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸Br), iodine-123 (¹²³I), iodine-125 (¹²⁵I), iodine-127 (¹²⁷I), iodine-129 (¹²⁹I), and iodine-131 (I). In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an “isotopic variant” of a compound is in an unstable form, that is, radioactive. In certain embodiments, an “isotopic variant” of a compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I). It will be understood that, in a compound as provided herein, any hydrogen can be ²H, for example, or any carbon can be ¹³C, as example, or any nitrogen can be ¹⁵N, as example, and any oxygen can be ¹⁸O, where feasible according to the judgment of one of skill. In certain embodiments, an “isotopic variant” of a compound contains unnatural proportions of deuterium.

The term “solvate” refers to a complex or aggregate formed by one or more molecules of a solute, e.g., a compound provided herein, and one or more molecules of a solvent, which present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The phrase “an isotopic variant thereof; or a pharmaceutically acceptable salt thereof; or a pharmaceutically acceptable solvate thereof” has the same meaning as the phrase “an isotopic variant of the compound referenced therein; or a pharmaceutically acceptable salt of the compound referenced therein; or a pharmaceutically acceptable salt of an isotopic variant of the compound referenced therein; or a pharmaceutically acceptable solvate of the compound referenced therein; or a pharmaceutically acceptable solvate of an isotopic variant of the compound referenced therein; or a pharmaceutically acceptable solvate of a pharmaceutically acceptable salt of the compound referenced therein; or a pharmaceutically acceptable solvate of a pharmaceutically acceptable salt of an isotopic variant of the compound referenced therein or its variant or its variant.”

### Compounds for Use in Spray-Dried Particles, Pharmaceutical Compositions, and Methods Provided Herein

In one embodiment, the compound for use in the spray-dried particles (also known as “spray-dried dispersion”), pharmaceutical compositions (e.g., granular formulations, tablet formulations, or capsule formulations), and methods provided herein is methyl N-[(1R)-2-[(2S)-2-[5-[4-[6-[2-[(2S)-1-[(2S)-2-(methoxycarbonylamino)-3-methyl-butanoyl]pyrrolidin-2-yl]-3H-benzimidazol-5-yl]thieno[3,2-b]thiophen-3-yl]phenyl]-1H-imidazol-2-yl]pyrrolidin-1-yl]-2-oxo-1-phenyl-ethyl]carbamate, having the structure of Formula A1 (“compound A1”);

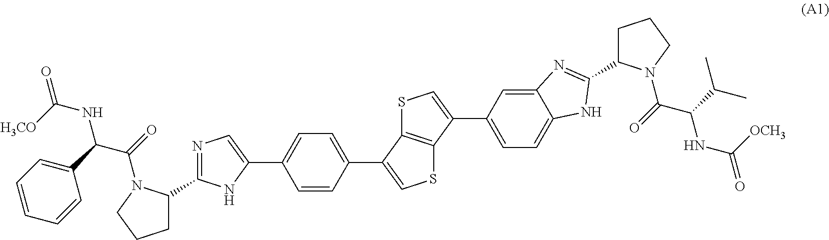

or [(S)-1-((S)-2-{5-[4-(6-{(S)-2-[1-((R)-2-methoxycarbonylamino-2-phenylacetyl)-pyrrolidin-2-yl]-3H-benzoimidazol-5-yl}-thieno[3,2-b]-thiophen-3-yl)-phenyl]-1H-imidazol-2-yl}pyrrolidine-1-carbonyl)-2-methylpropyl]-carbamic acid methyl ester, having the structure of Formula A2 (“compound A2”);

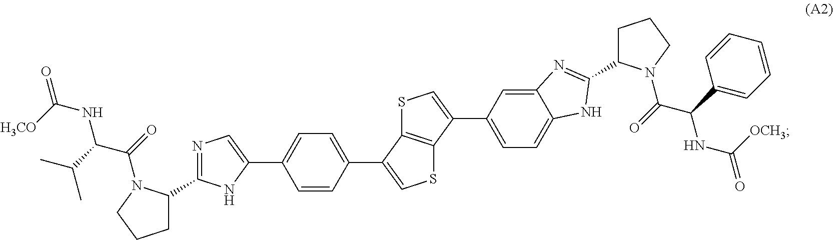

or an isotopic variant thereof, or a pharmaceutically acceptable salt thereof, or a pharmaceutically acceptable solvate thereof.

In one embodiment, the compound for use in the spray-dried particles, pharmaceutical compositions, and methods provided herein is compound A1 or an isotopic variant thereof, or a pharmaceutically acceptable salt thereof, or a pharmaceutically acceptable solvate thereof. In another embodiment, the compound provided herein is compound A1 or an isotopic variant thereof. In yet another embodiment, the compound provided herein is compound A1.

In one embodiment, the compound for use in the spray-dried particles, pharmaceutical compositions, and methods provided herein is compound A2 or an isotopic variant thereof, or a pharmaceutically acceptable salt thereof, or a pharmaceutically acceptable solvate thereof. In another embodiment, the compound provided herein is compound A2 or an isotopic variant thereof. In yet another embodiment, the compound provided herein is compound A2.

Compounds A1 and A2 are, inter alia, nonstructural protein 5A (NS5A) inhibitors. See U.S. Pat. No. 8,362,068; and U.S. Pat. App. Pub. No. US 2012/0252721; the disclosure of each of which is incorporated herein by reference in its entirety. For example, compound A1 is a potent and pan-genotypic inhibitor of HCV replication in vitro, e.g., having EC₅₀ values ranging from 2 to 24 pM against HCV genotypes 1a, 1b, 2a, 3a, 4a, and 5a. See U.S. Pat. App. Pub. No. US 2012/0252721, the disclosure of which is incorporated herein by reference in its entirety.

Compounds A1 and A2 can be prepared, for example, according to the methods described in U.S. Pat. No. 8,362,068. Compounds A1 and A2 can be also synthesized according to other methods apparent to those of skill in the art based upon the teaching herein.

In certain embodiments, the compound used in the spray-dried particles, pharmaceutical compositions (e.g., granular formulations, tablet formulations, or capsule formulations), and methods provided herein is compound A1 or A2, or a pharmaceutically solvate thereof. In certain embodiments, the compound used in the spray-dried particles, pharmaceutical compositions (e.g., granular formulations, tablet formulations, or capsule formulations), and methods provided herein is an isotopic variant of compound A1 or A2. In certain embodiments, the compound used in the spray-dried particles granulates, pharmaceutical compositions (e.g., granular formulations, tablet formulations, or capsule formulations), and methods provided herein is an isotopic variant of compound A1 or A2, or a pharmaceutically solvate thereof. In certain embodiments, the compound used in the spray-dried particles, granulates, pharmaceutical compositions (e.g., granular formulations, tablet formulations, or capsule formulations), and methods provided herein is a pharmaceutically acceptable salt of compound A1 or A2, or a pharmaceutically solvate thereof. In certain embodiments, the compound used in the spray-dried particles, granulates, pharmaceutical compositions (e.g., granular formulations, tablet formulations, or capsule formulations), and methods provided herein is a pharmaceutically acceptable salt of an isotopic variant of compound A1 or A2; or a pharmaceutically solvate thereof.

In certain embodiments, the compound used in the spray-dried particles, granulates, pharmaceutical compositions (e.g., granular formulations, tablet formulations, or capsule formulations), and methods provided herein is a pharmaceutically acceptable salt of compound A1 or A2, including, but not limited to, acetate, adipate, alginate, aspartate, benzoate, benzenesulfonate (besylate), bisulfate, butyrate, citrate, camphorate, camphorsulfonate, cyclopentanepropionate, digluconate, dodecylsulfate, 1,2-ethanedisulfonate (edisylate), ethanesulfonate (esylate), formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate (mesylate), 2-naphthalenesulfonate (napsylate), nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate, and undecanoate salts.

In certain embodiments, the compound used in the spray-dried particles, granulates, pharmaceutical compositions (e.g., granular formulations, tablet formulations, or capsule formulations), and methods provided herein is a solid. In certain embodiments, the compound used in the spray-dried particles, granulates, pharmaceutical compositions (e.g., granular formulations, tablet formulations, or capsule formulations), and methods provided herein is amorphous. In certain embodiments, the compound used in the spray-dried particles, granulates, pharmaceutical compositions (e.g., granular formulations, tablet formulations, or capsule formulations), and methods provided herein is crystalline.

As used herein, the compound used in the spray-dried particles, granulates, pharmaceutical compositions (e.g., granular formulations, tablet formulations, or capsule formulations), and methods provided herein is intended to encompass all possible stereoisomers, unless a particular stereochemistry is specified. Where structural isomers of the compound provided herein are interconvertible via a low energy barrier, the compound may exist as a single tautomer or a mixture of tautomers. This can take the form of proton tautomerism in the compound that contains, e.g., an imidazolyl or benzimidazolyl group; or so-called valence tautomerism in the compound that contain an aromatic moiety.

### Spray-Dried Particles

In one embodiment, provided herein are spray-dried particles comprising the compound provided herein (e.g., compound A1 or A2), or an isotopic variant thereof, or a pharmaceutically acceptable salt thereof, or a pharmaceutically acceptable solvate thereof; and a pharmaceutically acceptable excipient.

In one embodiment, the excipient is a polymer. In another embodiment, the excipient is a copolymer. In yet another embodiment, the excipient is a dispersant or carrier. In still another embodiment, the excipient is a carrier.

In certain embodiments, the excipient is a polyethylene glycol, a hydroxypropyl methylcellulose, a hypromellose phthalate, a hypromellose acetate succinate, a methacrylic acid and ethyl acrylate copolymer, a poloxamer, a tocopherol polyethylene glycol succinate, a polyvinyl pyrrolidone, or a mixture thereof.

In one embodiment, the excipient is a polyethylene glycol. In certain embodiments, the excipient is a polyethylene glycol having an average molecular weight of about 8,000 Da. In certain embodiments, the excipient is PEG 8000.

In another embodiment, the excipient is a hydroxypropyl methylcellulose. In certain embodiments, the excipient is HPMC 2910.

In yet another embodiment, the excipient is a hypromellose phthalate. In certain embodiments, the excipient is hypromellose phthalate having an average molecular weight of about 84,000 Da. In certain embodiments, the excipient is HPMPC, 55 grade.

In yet another embodiment, the excipient is a hypromellose acetate succinate. In certain embodiments, the excipient is a hypromellose acetate succinate having an average molecular weight of about 18,000 Da. In certain embodiments, the excipient is HPMCAS, MF grade.

In yet another embodiment, the excipient is a methacrylic acid and ethyl acrylate copolymer. In certain embodiments, the excipient is a methacrylic acid and ethyl acrylate copolymer having an average molecular weight of about 320,000 Da. In certain embodiments, the excipient is EUDRAGIT® L100-55.

In yet another embodiment, the excipient is a poloxamer. In certain embodiments, the excipient is a poloxamer having an average molecular weight of about 1,800 Da. In certain embodiments, the excipient is a poloxamer having an average molecular weight of about 4,000 Da. In certain embodiments, the excipient is Poloxamer 188. In certain embodiments, the excipient is Poloxamer 407.

In yet another embodiment, the excipient is a mixture of a tocopherol polyethylene glycol succinate and poloxamer. In certain embodiments, the excipient is a mixture of a tocopherol polyethylene glycol succinate and Poloxamer 188. In certain embodiments, the weight ratio of tocopherol the polyethylene glycol succinate to the poloxamer is ranging from about 0.1 to about 10, from about 0.2 to about 5, about 0.5 to about 2, or from about 0.75 to about 1.5. In certain embodiments, the weight ratio of the tocopherol polyethylene glycol succinate to the poloxamer is about 1.

In still another embodiment, the excipient is a polyvinyl pyrrolidone. In certain embodiments, the excipient is a polyvinyl pyrrolidone. In some embodiments, the polyvinyl pyrrolidone has an average molecular weight from about 30,000 Da to about 70,000 Da. In certain embodiments, the polyvinyl pyrrolidone has an average molecular weight from about 40,000 Da to about 60,000 Da. For example, the polyvinyl pyrrolidone can have an average molecular weight from about 40,000 Da to about 60,000 Da. In certain embodiments, the excipient is PVP-K30. In certain embodiments, the excipient is a polyvinyl pyrrolidone having an average molecular weight of about 360,000 Da. In certain embodiments, the excipient is PVP-K90.

In certain embodiments, the spray-dried particles provided herein comprise from about 5 to about 95%, from about 5 to about 50%, from about 10 to about 50%, from about 10 to about 40%, from about 20 to about 40%, or from about 20 to about 30% by weight of the compound provided herein (e.g., compound A1 or A2), or an isotopic variant thereof, or a pharmaceutically acceptable salt thereof, or a pharmaceutically acceptable solvate thereof.

In certain embodiments, the spray-dried particles provided herein comprise about 5%, about 10%, about 15%, about 20%, about 25%, about 30%, about 35%, about 40%, about 45%, or about 50% by weight of the compound provided herein (e.g., compound A1 or A2), or an isotopic variant thereof, or a pharmaceutically acceptable salt thereof, or a pharmaceutically acceptable solvate thereof. In certain embodiments, the spray-dried particles provided herein comprise about 20%, about 21%, about 22%, about 23%, about 24%, about 25%, about 26%, about 27%, about 28%, about 29%, or about 30% by weight of the compound provided herein (e.g., compound A1 or A2), or an isotopic variant thereof, or a pharmaceutically acceptable salt thereof, or a pharmaceutically acceptable solvate thereof.

In certain embodiments, the spray-dried particles provided herein comprise from about 5 to about 95%, from about 50 to about 95%, from about 50 to about 90%, from about 60 to about 90%, from about 60 to about 80%, or from about 70 to about 80% by weight of an excipient. In certain embodiments, the spray-dried particles provided herein comprise about 95%, about 90%, about 85%, about 80%, about 75%, about 70%, about 65%, about 60%, about 55%, or about 50% by weight of an excipient. In certain embodiments, the spray-dried particles provided herein comprise about 80%, about 79%, about 78%, about 77%, about 76%, about 75%, about 74%, about 73%, about 72%, about 71%, or about 70% by weight of an excipient.

In certain embodiments, the spray-dried particles provided herein have an average particle size ranging from about 1 to about 100 μm, from about 1 to about 60 μm, from about 1 to about 50 μm, from about 10 to about 50 m, from about 10 to about 30 μm, or from about 10 to about 25 μm. In certain embodiments, the spray-dried particles have an average particle size ranging from about 10 to about 30 μm. In certain embodiments, the spray-dried particles have an average particle size ranging from about 10 to about 25 μm.

In certain embodiments, the spray-dried particles provided herein have a bulk density ranging from about 0.01 to about 1 g/mL, from about 0.05 to about 0.5 g/mL, from about 0.05 to about 0.3 g/mL, from about 0.1 to about 0.5 g/mL, from about 0.1 to about 0.3 g/mL, or from about 0.2 to about 0.3. In certain embodiments, the spray-dried particles have a bulk density of about 0.1, about 0.12, about 0.14, about 0.16, about 0.18, about 0.20, about 0.22, about 0.24, about 0.25, about 0.26, about 0.28, or about 0.3 g/mL. In certain embodiments, the spray-dried particles have a bulk density ranging from about 0.2 to about 1 g/mL, from about 0.2 to about 0.8 g/mL, or from about 0.2 to about 0.6 g/mL. In certain embodiments, the spray-dried particles have a bulk density of about 0.25 g/mL. In certain embodiments, the bulk density of the spray-dried particles provided herein is quantitated according to Method 616 in USP XXVI (2003).

In certain embodiments, the spray-dried particles provided herein have a tapped density ranging from about 0.05 to about 1 g/mL, from about 0.1 to about 1 g/mL, from about 0.2 to about 0.8 g/mL, from about 0.2 to about 0.6 g/mL, from about 0.4 to about 0.60 g/mL, or from about 0.5 to about 0.60 g/mL. In certain embodiments, the spray-dried particles have a tapped density of about 0.2, about 0.25, about 0.3, about 0.35, about 0.4, about 0.45, about 0.5, about 0.55, or about 0.6 g/mL. In certain embodiments, the spray-dried particles have a tapped density of about 0.5 g/mL. In certain embodiments, the tapped density of the spray-dried particles provided herein is quantitated according to Method 616 in USP XXVI (2003).

In certain embodiments, the spray-dried particles provided herein have a Carr index ranging from about 5 to about 50, from about 10 to about 50, from about 20 to about 40, or from about 20 to about 30. In certain embodiments, the spray-dried particles provided herein have a Carr index of about 20, about 21, about 22, about 23, about 24, about 25, about 26, about 27, about 28, about 29, about 30, about 31, about 32, about 33, about 34, or about 35. In certain embodiments, the spray-dried particles provided herein have a Carr index of about 25.

In certain embodiments provided herein, the spray-dried particles provided herein have a residual water content of no greater than about 40%, no greater than about 30%, no greater than about 20%, no greater than about 10%, no greater than about 9%, no greater than about 8%, no greater than about 7%, no greater than about 6%, no greater than about 5%, no greater than about 4%, or no greater than about 3% by weight. In a certain embodiment, the spray-dried particles have a residual water content of no greater than about 6% by weight. In a certain embodiment, the spray-dried particles have a residual water content ranging from about 1 to about 30% by weight. In a certain embodiment, the spray-dried particles have a residual water content ranging from about 1 to about 20% by weight. In a certain embodiment, the spray-dried particles have a residual water content ranging from about 2 to about 9% by weight. In a certain embodiment, the spray-dried particles have a residual water content ranging from about 4 to about 10% by weight. In a certain embodiment, the spray-dried particles have a residual water content ranging from about 4 to about 6% by weight. In certain embodiments, the residual water content of the spray-dried particles provided herein is quantitated according to Method 919 in USP XXVI (2003).

In certain embodiments, the spray-dried particles provided herein have a residual organic solvent content of no greater than about 5,000 ppm, no greater than about 4,000 ppm, no greater than about 3,000 ppm, no greater than about 2,000 ppm, no greater than about 1,000 ppm, no greater than about 900 ppm, no greater than about 800 ppm, no greater than about 700 ppm, no greater than about 600 ppm, no greater than about 500 ppm, no greater than about 400 ppm, no greater than about 300 ppm, no greater than about 200 ppm, or no greater than about 100 ppm. In a certain embodiment, the spray-dried particles have a residual organic solvent content of no greater than about 3,000 ppm. In a certain embodiment, the spray-dried particles have a residual organic solvent content of no greater than about 800 ppm. In certain embodiments, the organic solvent is tetrahydrofuran (THF). In certain embodiments, the solvent is acetone. In certain embodiments, the solvent is an alcohol. In certain embodiments, the solvent is ethanol. In certain embodiments, the solvent is methanol. In certain embodiments, the residual organic solvent content of the spray-dried particles provided herein is quantitated according to Method 467 in USP XXVI (2003).

In certain embodiments, the spray-dried particles provided herein have a residual methanol content of no greater than about 5,000 ppm, no greater than about 4,000 ppm, no greater than about 3,000 ppm, no greater than about 2,000 ppm, no greater than about 1,000 ppm, no greater than about 900 ppm, no greater than about 800 ppm, no greater than about 700 ppm, no greater than about 600 ppm, no greater than about 500 ppm, no greater than about 400 ppm, no greater than about 300 ppm, no greater than about 200 ppm, or no greater than about 100 ppm. In certain embodiments, the spray-dried particles provided herein have a residual methanol content of no greater than about 3,000 ppm.

In certain embodiments, the spray-dried particles provided herein have a residual THF content of no greater than about 1,000 ppm, no greater than about 900 ppm, no greater than about 800 ppm, no greater than about 700 ppm, no greater than about 600 ppm, no greater than about 500 ppm, no greater than about 400 ppm, no greater than about 300 ppm, no greater than about 200 ppm, or no greater than about 100 ppm. In certain embodiments, the spray-dried particles provided herein have a residual THF content of no greater than about 800 ppm.

In certain embodiments, the spray-dried particles provided herein are stable at a 25° C. and 60% relative humidity for a period ranging from about 6 months to about 10 years, from about 6 months to about 5 years, from about 6 months to about 2 years, from about 12 months to about 24 months. In certain embodiments, the spray-dried particles provided herein are stable at a 25° C. and 60% relative humidity for a period from about 6 months to about 12 months, from about 6 months to about 18 months, or from about 6 months to about 24 months. In certain embodiments, the spray-dried particles provided herein are stable at a 25° C. and 60% relative humidity for at least 6 months, at least 12 months, at least 18 months, at least 24 months, or at least 5 years.

In certain embodiments, the compound (e.g., compound A1 or A2), or an isotopic variant thereof, or a pharmaceutically acceptable salt, or a pharmaceutically acceptable solvate thereof, used in preparing the spray-dried particles provided herein is amorphous. In certain embodiments, the compound (e.g., compound A1 or A2), or an isotopic variant thereof, or a pharmaceutically acceptable salt, or a pharmaceutically acceptable solvate thereof, used in preparing the spray-dried particles provided herein is crystalline.

In certain embodiments, the compound (e.g., compound A1 or A2), or an isotopic variant thereof, or a pharmaceutically acceptable salt, or a pharmaceutically acceptable solvate thereof, which is comprised in the spray-dried particles provided herein, is amorphous. In certain embodiments, the compound (e.g., compound A1 or A2), or an isotopic variant thereof, or a pharmaceutically acceptable salt, or a pharmaceutically acceptable solvate thereof, which is comprised in the spray-dried particles provided herein, is crystalline.

In one embodiment, the spray-dried particles provided herein comprise from about 5 to about 50% by weight of the compound provided herein (e.g., compound A1 or A2), or an isotopic variant thereof, or a pharmaceutically acceptable salt, or a pharmaceutically acceptable solvate thereof; and from about 50 to about 95% by weight of an excipient.

In another embodiment, the spray-dried particles provided herein comprise from about 10 to about 30% by weight of the compound provided herein (e.g., compound A1 or A2), or an isotopic variant thereof, or a pharmaceutically acceptable salt, or a pharmaceutically acceptable solvate thereof; and from about 70 to about 90% by weight of an excipient.

In yet another embodiment, the spray-dried particles provided herein comprise about 25% by weight of the compound provided herein (e.g., compound A1 or A2), or an isotopic variant thereof, or a pharmaceutically acceptable salt, or a pharmaceutically acceptable solvate thereof; and about 75% by weight of an excipient.

In still another embodiment, the spray-dried particles provided herein comprise about 20% by weight of the compound provided herein (e.g., compound A1 or A2), or an isotopic variant thereof, or a pharmaceutically acceptable salt, or a pharmaceutically acceptable solvate thereof; and about 80% by weight of an excipient.

In one embodiment, the spray-dried particles provided herein comprise the compound provided herein (e.g., compound A1 or A2), or an isotopic variant thereof, or a pharmaceutically acceptable salt, or a pharmaceutically acceptable solvate thereof; and a polyvinyl pyrrolidine, in one embodiment, PVP-K30.

In another embodiment, the spray-dried particles provided herein comprise from about 5 to about 50% by weight of the compound provided herein (e.g., compound A1 or A2), or an isotopic variant thereof, or a pharmaceutically acceptable salt, or a pharmaceutically acceptable solvate thereof; and from about 50 to about 95% by weight of a polyvinyl pyrrolidine, in one embodiment, PVP-K30.

In yet another embodiment, the spray-dried particles provided herein comprise from about 10 to about 30% by weight of the compound provided herein (e.g., compound A1 or A2), or an isotopic variant thereof, or a pharmaceutically acceptable salt, or a pharmaceutically acceptable solvate thereof; and from about 70 to about 90% by weight of a polyvinyl pyrrolidine, in one embodiment, PVP-K30.

In still another embodiment, the spray-dried particles provided herein comprise about 25% by weight of the compound provided herein (e.g., compound A1 or A2), or an isotopic variant thereof, or a pharmaceutically acceptable salt, or a pharmaceutically acceptable solvate thereof; and about 75% by weight of a polyvinyl pyrrolidine, in one embodiment, PVP-K30.

In certain embodiments, the PVP-K30-containing spray-dried particles provided herein have an average particle size ranging from about 15 μm to about 20 μm. In certain embodiments, the PVP-K30-containing spray-dried particles provided herein have a bulk density of about 0.25 g/mL. In certain embodiments, the PVP-K30-containing spray-dried particles provided herein have a tapped density of about 0.5 g/mL. In certain embodiments, the PVP-K30-containing spray-dried particles provided herein have a residual water content of no greater than about 6% by weight. In certain embodiments, the PVP-K30-containing spray-dried particles provided herein have a residual methanol content of no greater than about 3,000 ppm. In certain embodiments, the PVP-K30-containing spray-dried particles provided herein have a residual THF content of no greater than about 800 ppm.

In one embodiment, the spray-dried particles provided herein comprise the compound provided herein (e.g., compound A1 or A2), or an isotopic variant thereof, or a pharmaceutically acceptable salt, or a pharmaceutically acceptable solvate thereof; and a polyethylene glycol, in one embodiment, PEG 8000.

In another embodiment, the spray-dried particles provided herein comprise from about 5 to about 50% by weight of the compound provided herein (e.g., compound A1 or A2), or an isotopic variant thereof, or a pharmaceutically acceptable salt, or a pharmaceutically acceptable solvate thereof; and from about 50 to about 95% by weight a polyethylene glycol, in one embodiment, PEG 8000.

In yet another embodiment, the spray-dried particles provided herein comprise from about 10 to about 30% by weight of the compound provided herein (e.g., compound A1 or A2), or an isotopic variant thereof, or a pharmaceutically acceptable salt, or a pharmaceutically acceptable solvate thereof; and from about 70 to about 90% by weight of a polyethylene glycol, in one embodiment, PEG 8000.

In still another embodiment, the spray-dried particles provided herein comprise about 20% by weight of the compound provided herein (e.g., compound A1 or A2), or an isotopic variant thereof, or a pharmaceutically acceptable salt, or a pharmaceutically acceptable solvate thereof; and about 80% by weight of a polyethylene glycol, in one embodiment, PEG 8000.

In one embodiment, the spray-dried particles provided herein comprise the compound provided herein (e.g., compound A1 or A2), or an isotopic variant thereof, or a pharmaceutically acceptable salt, or a pharmaceutically acceptable solvate thereof; and a hydroxypropyl methyl cellulose, in one embodiment, HPMC 2910.

In another embodiment, the spray-dried particles provided herein comprise from about 5 to about 50% by weight of the compound provided herein (e.g., compound A1 or A2), or an isotopic variant thereof, or a pharmaceutically acceptable salt, or a pharmaceutically acceptable solvate thereof; and from about 50 to about 95% by weight of a hydroxypropyl methyl cellulose, in one embodiment, HPMC 2910.

In yet another embodiment, the spray-dried particles provided herein comprise from about 10 to about 30% by weight of the compound provided herein (e.g., compound A1 or A2), or an isotopic variant thereof, or a pharmaceutically acceptable salt, or a pharmaceutically acceptable solvate thereof; and from about 70 to about 90% by weight of a hydroxypropyl methyl cellulose, in one embodiment, HPMC 2910.

In still another embodiment, the spray-dried particles provided herein comprise about 20% by weight of the compound provided herein (e.g., compound A1 or A2), or an isotopic variant thereof, or a pharmaceutically acceptable salt, or a pharmaceutically acceptable solvate thereof; and about 80% by weight of a hydroxypropyl methyl cellulose, in one embodiment, HPMC 2910.

In one embodiment, the spray-dried particles provided herein comprise the compound provided herein (e.g., compound A1 or A2), or an isotopic variant thereof, or a pharmaceutically acceptable salt, or a pharmaceutically acceptable solvate thereof; and a hydroxypropyl methyl cellulose phthalate, in one embodiment, HPMCP, 55 grade.

In another embodiment, the spray-dried particles provided herein comprise from about 5 to about 50% by weight of the compound provided herein (e.g., compound A1 or A2), or an isotopic variant thereof, or a pharmaceutically acceptable salt, or a pharmaceutically acceptable solvate thereof; and from about 50 to about 95% by weight and a hydroxypropyl methyl cellulose phthalate, in one embodiment, HPMCP, 55 grade.

In yet another embodiment, the spray-dried particles provided herein comprise from about 10 to about 30% by weight of the compound provided herein (e.g., compound A1 or A2), or an isotopic variant thereof, or a pharmaceutically acceptable salt, or a pharmaceutically acceptable solvate thereof; and from about 70 to about 90% by weight of and a hydroxypropyl methyl cellulose phthalate, in one embodiment, HPMCP, 55 grade.

In still another embodiment, the spray-dried particles provided herein comprise about 20% by weight of the compound provided herein (e.g., compound A1 or A2), or an isotopic variant thereof, or a pharmaceutically acceptable salt, or a pharmaceutically acceptable solvate thereof; and about 80% by weight of and a hydroxypropyl methyl cellulose phthalate, in one embodiment, HPMCP, 55 grade.

In one embodiment, the spray-dried particles provided herein comprise the compound provided herein (e.g., compound A1 or A2), or an isotopic variant thereof, or a pharmaceutically acceptable salt, or a pharmaceutically acceptable solvate thereof; and a hydroxypropyl methylcellulose acetate succinate, in one embodiment, HPMCAS, MF grade.

In another embodiment, the spray-dried particles provided herein comprise from about 5 to about 50% by weight of the compound provided herein (e.g., compound A1 or A2), or an isotopic variant thereof, or a pharmaceutically acceptable salt, or a pharmaceutically acceptable solvate thereof; and from about 50 to about 95% by weight and a hydroxypropyl methylcellulose acetate succinate, in one embodiment, HPMCAS, MF grade.

In yet another embodiment, the spray-dried particles provided herein comprise from about 10 to about 30% by weight of the compound provided herein (e.g., compound A1 or A2), or an isotopic variant thereof, or a pharmaceutically acceptable salt, or a pharmaceutically acceptable solvate thereof; and from about 70 to about 90% by weight of and a hydroxypropyl methylcellulose acetate succinate, in one embodiment, HPMCAS, MF grade.

In still another embodiment, the spray-dried particles provided herein comprise about 20% by weight of the compound provided herein (e.g., compound A1 or A2), or an isotopic variant thereof, or a pharmaceutically acceptable salt, or a pharmaceutically acceptable solvate thereof; and about 80% by weight of and a hydroxypropyl methylcellulose acetate succinate, in one embodiment, HPMCAS, MF grade.

In one embodiment, the spray-dried particles provided herein comprise the compound provided herein (e.g., compound A1 or A2), or an isotopic variant thereof, or a pharmaceutically acceptable salt, or a pharmaceutically acceptable solvate thereof; and a poloxamer, in one embodiment, Poloxamer 188, in another embodiment, Poloxamer 407.

In another embodiment, the spray-dried particles provided herein comprise from about 5 to about 50% by weight of the compound provided herein (e.g., compound A1 or A2), or an isotopic variant thereof, or a pharmaceutically acceptable salt, or a pharmaceutically acceptable solvate thereof; and from about 50 to about 95% by weight and a poloxamer, in one embodiment, Poloxamer 188, in another embodiment, Poloxamer 407.

In yet another embodiment, the spray-dried particles provided herein comprise from about 10 to about 30% by weight of the compound provided herein (e.g., compound A1 or A2), or an isotopic variant thereof, or a pharmaceutically acceptable salt, or a pharmaceutically acceptable solvate thereof; and from about 70 to about 90% by weight of and a poloxamer, in one embodiment, Poloxamer 188, in another embodiment, Poloxamer 407.

In still another embodiment, the spray-dried particles provided herein comprise about 20% by weight of the compound provided herein (e.g., compound A1 or A2), or an isotopic variant thereof, or a pharmaceutically acceptable salt, or a pharmaceutically acceptable solvate thereof; and about 80% by weight of and a poloxamer, in one embodiment, Poloxamer 188, in another embodiment, Poloxamer 407.

In one embodiment, the spray-dried particles provided herein comprise the compound provided herein (e.g., compound A1 or A2), or an isotopic variant thereof, or a pharmaceutically acceptable salt, or a pharmaceutically acceptable solvate thereof; and a polyvinyl pyrrolidone, in one embodiment, PVP K-90.

In another embodiment, the spray-dried particles provided herein comprise from about 5 to about 50% by weight of the compound provided herein (e.g., compound A1 or A2), or an isotopic variant thereof, or a pharmaceutically acceptable salt, or a pharmaceutically acceptable solvate thereof; and from about 50 to about 95% by weight and a polyvinyl pyrrolidone, in one embodiment, PVP K-90.

In yet another embodiment, the spray-dried particles provided herein comprise from about 10 to about 30% by weight of the compound provided herein (e.g., compound A1 or A2), or an isotopic variant thereof, or a pharmaceutically acceptable salt, or a pharmaceutically acceptable solvate thereof; and from about 70 to about 90% by weight of a polyvinyl pyrrolidone, in one embodiment, PVP K-90.

In still another embodiment, the spray-dried particles provided herein comprise about 20% by weight of the compound provided herein (e.g., compound A1 or A2), or an isotopic variant thereof, or a pharmaceutically acceptable salt, or a pharmaceutically acceptable solvate thereof; and about 80% by weight of a polyvinyl pyrrolidone, in one embodiment, PVP K-90.

In one embodiment, the spray-dried particles provided herein comprise the compound provided herein (e.g., compound A1 or A2), or an isotopic variant thereof, or a pharmaceutically acceptable salt, or a pharmaceutically acceptable solvate thereof; and a methacrylic acid and ethyl acrylate copolymer, in one embodiment, EUDRAGIT® L100-55.

In another embodiment, the spray-dried particles provided herein comprise from about 5 to about 50% by weight of the compound provided herein (e.g., compound A1 or A2), or an isotopic variant thereof, or a pharmaceutically acceptable salt, or a pharmaceutically acceptable solvate thereof; and from about 50 to about 95% by weight and a methacrylic acid and ethyl acrylate copolymer, in one embodiment, EUDRAGIT® L100-55.

In yet another embodiment, the spray-dried particles provided herein comprise from about 10 to about 30% by weight of the compound provided herein (e.g., compound A1 or A2), or an isotopic variant thereof, or a pharmaceutically acceptable salt, or a pharmaceutically acceptable solvate thereof; and from about 70 to about 90% by weight of a methacrylic acid and ethyl acrylate copolymer, in one embodiment, EUDRAGIT® L100-55.

In still another embodiment, the spray-dried particles provided herein comprise about 20% by weight of the compound provided herein (e.g., compound A1 or A2), or an isotopic variant thereof, or a pharmaceutically acceptable salt, or a pharmaceutically acceptable solvate thereof; and about 80% by weight of a methacrylic acid and ethyl acrylate copolymer, in one embodiment, EUDRAGIT® L100-55.

In one embodiment, the spray-dried particles provided herein comprise the compound provided herein (e.g., compound A1 or A2), or an isotopic variant thereof, or a pharmaceutically acceptable salt, or a pharmaceutically acceptable solvate thereof; and a mixture of a tocopherol polyethylene glycol succinate (TPGS) and a poloxamer, in one embodiment, Poloxamer 188. In certain embodiments, the weight ratio of the tocopherol polyethylene glycol succinate (TPGS) and the poloxamer is about 1.

In another embodiment, the spray-dried particles provided herein comprise from about 5 to about 50% by weight of the compound provided herein (e.g., compound A1 or A2), or an isotopic variant thereof, or a pharmaceutically acceptable salt, or a pharmaceutically acceptable solvate thereof; and from about 50 to about 95% by weight and a mixture of a TPGS and a poloxamer, in one embodiment, Poloxamer 188.

In yet another embodiment, the spray-dried particles provided herein comprise from about 10 to about 30% by weight of the compound provided herein (e.g., compound A1 or A2), or an isotopic variant thereof, or a pharmaceutically acceptable salt, or a pharmaceutically acceptable solvate thereof; and from about 70 to about 90% by weight of a mixture of a TPGS and a poloxamer, in one embodiment, Poloxamer 188.

In still another embodiment, the spray-dried particles provided herein comprise about 20% by weight of the compound provided herein (e.g., compound A1 or A2), or an isotopic variant thereof, or a pharmaceutically acceptable salt, or a pharmaceutically acceptable solvate thereof; and about 80% by weight of a mixture of a TPGS and a poloxamer, in one embodiment, Poloxamer 188.

In one embodiment, the spray-dried particles provided herein are made from a solution that does not contain tetrahydrofuran. In another embodiment, the spray-dried particles provided herein are made from an aqueous solution.

### Pharmaceutical Compositions

In one embodiment, the pharmaceutical composition provided herein comprises from about 1 to about 50% by weight of the compound provided herein (e.g., compound A1 or A2), or an isotopic variant thereof, or a pharmaceutically acceptable salt thereof, or a pharmaceutically acceptable solvate thereof; and from about 50 to about 99% by weight of an excipient.

In certain embodiments, the pharmaceutical composition provided herein comprises from about 1 to about 50%, from about 2 to about 25%, from about 5 to about 20%, from about 5 to about 15%, or from about 5 to about 10% by weight of the compound provided herein (e.g., compound A1 or A2), or an isotopic variant thereof, or a pharmaceutically acceptable salt thereof, or a pharmaceutically acceptable solvate thereof. In certain embodiments, the pharmaceutical composition provided herein comprises about 5%, about 5.5%, about 6%, about 6.5%, about 7%, about 7.5%, about 8%, about 8.5%, about 9%, about 9.5%, or about 10% by weight of the compound provided herein (e.g., compound A1 or A2), or an isotopic variant thereof, or a pharmaceutically acceptable salt thereof, or a pharmaceutically acceptable solvate thereof. In certain embodiments, the pharmaceutical composition comprises from about 6 to about 9% by weight of the compound provided herein (e.g., compound A1 or A2), or an isotopic variant thereof, or a pharmaceutically acceptable salt thereof, or a pharmaceutically acceptable solvate thereof. In certain embodiments, the pharmaceutical composition comprises from about 6 to about 6.5% by weight of the compound provided herein (e.g., compound A1 or A2), or an isotopic variant thereof, or a pharmaceutically acceptable salt thereof, or a pharmaceutically acceptable solvate thereof. In certain embodiments, the pharmaceutical composition comprises from about 8 to about 8.5% by weight of the compound provided herein (e.g., compound A1 or A2), or an isotopic variant thereof, or a pharmaceutically acceptable salt thereof, or a pharmaceutically acceptable solvate thereof.

In certain embodiments, the pharmaceutical composition provided herein comprises from about 50 to about 99%, from about 75 to about 98%, from about 80 to about 95%, from about 85 to about 95%, or from about 90 to about 95% by weight of an excipient. In certain embodiments, the pharmaceutical composition comprises about 90%, about 91%, about 92%, about 93%, about 94%, about 95%, about 96%, about 97%, about 98%, or about 99% by weight of an excipient. In certain embodiments, the pharmaceutical composition comprises from about 91 to about 94% by weight of an excipient. In certain embodiments, the pharmaceutical composition comprises from about 91.5 to about 92% by weight of an excipient.

In one embodiment, the pharmaceutical composition provided herein comprises from about 5 to about 20% by weight of the compound provided herein (e.g., compound A1 or A2), or an isotopic variant thereof, or a pharmaceutically acceptable salt thereof, or a pharmaceutically acceptable solvate thereof; and from about 80 to about 95% by weight of an excipient.

In another embodiment, the pharmaceutical composition provided herein comprises from about 5 to about 10% by weight of the compound provided herein (e.g., compound A1 or A2), or an isotopic variant thereof, or a pharmaceutically acceptable salt thereof, or a pharmaceutically acceptable solvate thereof; and from about 90 to about 95% by weight of an excipient.

In yet another embodiment, the pharmaceutical composition provided herein comprises from about 6 to about 9% by weight of the compound provided herein (e.g., compound A1 or A2), or an isotopic variant thereof, or a pharmaceutically acceptable salt thereof, or a pharmaceutically acceptable solvate thereof; and from about 91 to about 94% by weight of an excipient.

In one embodiment, provided herein is a pharmaceutical composition comprising (i) the compound provided herein (e.g., compound A1 or A2), or an isotopic variant thereof, or a pharmaceutically acceptable salt, or a pharmaceutically acceptable solvate thereof; and (ii) a dispersant or carrier, a disintegrant, a filler, a glidant, a lubricant, an organic acid, a surfactant, or a mixture thereof.

In another embodiment, provided herein is a pharmaceutical composition comprising (i) the compound provided herein (e.g., compound A1 or A2), or an isotopic variant thereof, or a pharmaceutically acceptable salt thereof, or a pharmaceutically acceptable solvate thereof; and (ii) a dispersant or carrier, a disintegrant, a filler, a glidant, and a lubricant.

In yet another embodiment, provided herein is a pharmaceutical composition comprising (i) the compound provided herein (e.g., compound A1 or A2), or an isotopic variant thereof, or a pharmaceutically acceptable salt thereof, or a pharmaceutically acceptable solvate thereof; and (ii) a dispersant or carrier, a disintegrant, a filler, a glidant, a lubricant, and an organic acid.

In yet another embodiment, provided herein is a pharmaceutical composition comprising (i) the compound provided herein (e.g., compound A1 or A2), or an isotopic variant thereof, or a pharmaceutically acceptable salt thereof, or a pharmaceutically acceptable solvate thereof; and (ii) a dispersant or carrier, a disintegrant, a filler, a glidant, a lubricant, and a surfactant.

In still another embodiment, provided herein is a pharmaceutical composition comprising (i) the compound provided herein (e.g., compound A1 or A2), or an isotopic variant thereof, or a pharmaceutically acceptable salt thereof, or a pharmaceutically acceptable solvate thereof; and (ii) a dispersant or carrier, a disintegrant, a filler, a glidant, a lubricant, an organic acid, and a surfactant.

In certain embodiments, the pharmaceutical composition provided herein comprises from about 5 to about 50%, from about 10 to about 50%, from about 15 to about 30%, or from about 15 to about 25% by weight of a dispersant or carrier. In certain embodiments, the pharmaceutical composition provided herein comprises about 15%, about 16%, about 17%, about 18%, about 19%, about 20%, about 21%, about 22%, about 23%, about 24%, about 25%, about 26%, about 27%, about 28%, about 29%, or about 30% by weight of a dispersant or carrier. In certain embodiments, the pharmaceutical composition provided herein comprises about 19% by weight of a dispersant or carrier. In certain embodiments, the pharmaceutical composition provided herein comprises about 25% by weight of a dispersant or carrier.

In certain embodiments, the dispersant or carrier is a hypromellose, a hypromellose acetate succinate, a hypromellose phthalate, a methacrylic acid and ethyl acrylate copolymer, a poloxamer, a polyethylene glycol, a tocopherol polyethylene glycol succinate, or a mixture thereof. In certain embodiments, the dispersant or carrier is a hypromellose acetate succinate. In certain embodiments, the dispersant or carrier is a hypromellose acetate succinate having an average molecular weight of about 18,000 Da. In certain embodiments, the dispersant or carrier is HPMCAS, MF grade.

In certain embodiments, the pharmaceutical composition provided herein comprises from about 1 to about 50%, from about 2 to about 20%, from about 0.5 to about 15%, or from about 5 to about 15% by weight of a disintegrant. In certain embodiments, the pharmaceutical composition provided herein comprises about 5%, about 6%, about 7%, about 8%, about 9%, about 10%, about 11%, about 12%, about 13%, about 14%, or about 15% by weight of a disintegrant. In certain embodiments, the pharmaceutical composition provided herein comprises about 6% by weight of a disintegrant. In certain embodiments, the pharmaceutical composition provided herein comprises about 10% by weight of a disintegrant. In certain embodiments, the disintegrant is a crosslinked polyvinyl pyrrolidone or croscarmellose sodium. In certain embodiments, the disintegrant is a crosslinked polyvinyl pyrrolidone. In certain embodiments, the disintegrant is POLYPLASDONE® XL. In certain embodiments, the disintegrant is croscarmellose sodium. In certain embodiments, the disintegrant is AC-DI-SOL®.

In certain embodiments, the pharmaceutical composition provided herein comprises from about 10 to about 95%, from about 20 to about 90%, from about 25 to about 75%, or from about 30 to about 70% by weight of a filler. In certain embodiments, the pharmaceutical composition provided herein comprises about 25%, about 30%, about 35%, about 40%, about 45%, about 50%, about 55%, about 60%, about 65%, about 70%, or about 75% by weight of a filler. In certain embodiments, the pharmaceutical composition provided herein comprises about 30%, about 50%, or about 65% by weight of a filler. In certain embodiments, the filler is a microcrystalline cellulose, lactose, or a mixture thereof. In certain embodiments, the filler is a microcrystalline cellulose. In certain embodiments, the filler is AVICEL® PH 102. In certain embodiments, the filler is lactose. In certain embodiments, the filler is lactose FAST FLO® 316. In certain embodiments, the filler is a mixture of microcrystalline cellulose and lactose. In certain embodiments, the filler is a mixture of AVICEL® PH 102 and lactose FAST FLO® 316.

In certain embodiments, the pharmaceutical composition provided herein comprises from about 0.1 to about 5%, from about 0.1 to about 2%, from about 0.2 to about 1.5%, or from about 0.5 to about 1% by weight of a glidant. In certain embodiments, the pharmaceutical composition provided herein comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% by weight of a glidant. In certain embodiments, the pharmaceutical composition provided herein comprises from about 0.5 to about 1.5% by weight of a glidant. In certain embodiments, the glidant is a colloidal silicon dioxide. In certain embodiment, the glidant is CAB-O-SIL®.

In certain embodiments, the pharmaceutical composition provided herein comprises from about 0.05 to about 5%, from about 0.1 to about 2%, or from about 0.2 to about 1% by weight of a lubricant. In certain embodiments, the pharmaceutical composition provided herein comprises about 0.1%, about 0.2%, about 0.3%, about 0.4%, about 0.5%, about 0.6%, about 0.7%, about 0.8%, about 0.9%, or about 1% by weight of a lubricant. In certain embodiments, the pharmaceutical composition provided herein comprises about 0.5% by weight of a lubricant. In certain embodiments, the lubricant is magnesium stearate.

In certain embodiments, the pharmaceutical composition provided herein comprises from about 1 to about 25%, from about 2 to about 20%, from about 5 to about 15%, or from about 10 to about 15% by weight of an organic acid. In certain embodiments, the pharmaceutical composition provided herein comprises about 5%, about 6%, about 7%, about 8%, about 9%, about 10%, about 11%, about 12%, about 13%, about 14%, or about 15% by weight of an organic acid. In certain embodiments, the pharmaceutical composition provided herein comprises from about 10 to about 15% by weight of an organic acid. In certain embodiments, the organic acid is tartaric acid.

In certain embodiments, the pharmaceutical composition provided herein comprises from about 1 to about 30%, from about 2 to about 25%, or from about 5 to about 25% by weight of a surfactant. In certain embodiments, the pharmaceutical composition provided herein comprises about 10%, about 11%, about 12%, about 13%, about 14%, about 15%, about 16%, about 17%, about 18%, about 19%, or about 20% by weight of a surfactant. In certain embodiments, the pharmaceutical composition provided herein comprises from about 10 to about 20% by weight of a surfactant. In certain embodiments, the surfactant is sodium lauryl sulfate.

In one embodiment, the pharmaceutical composition provided herein comprises (i) from about 5 to about 25% of the compound provided herein (e.g., compound A1 or A2), or an isotopic variant thereof, or a pharmaceutically acceptable salt thereof, or a pharmaceutically acceptable solvate thereof; and (ii) from about 10 to about 25% by weight of a dispersant or carrier, from about 5 to about 25% by weight of a disintegrant, from about 25 to about 80% by weight of a filler, from about 0.1 to about 5% by weight of a glidant, and from about 0.05 to about 2% by weight of a lubricant.

In another embodiment, the pharmaceutical composition provided herein comprises (i) from about 5 to about 10% of the compound provided herein (e.g., compound A1 or A2), or an isotopic variant thereof, or a pharmaceutically acceptable salt thereof, or a pharmaceutically acceptable solvate thereof; and (ii) from about 15 to about 25% by weight of a dispersant or carrier, from about 5 to about 15% by weight of a disintegrant, from about 30 to about 70% by weight of a filler, from about 0.5 to about 1.5% by weight of a glidant, and from about 0.1 to about 1% by weight of a lubricant.

In yet another embodiment, the pharmaceutical composition provided herein comprises (i) from about 6 to about 9% by weight of the compound provided herein (e.g., compound A1 or A2), or an isotopic variant thereof, or a pharmaceutically acceptable salt thereof, or a pharmaceutically acceptable solvate thereof; and (ii) from about 18 to about 25% by weight of a dispersant or carrier, from about 6 to about 10% by weight of a disintegrant, from about 30 to about 70% by weight of a filler, from about 0.5 to about 1.5% by weight of a glidant, and from about 0.3 to about 0.7% by weight of a lubricant.

In still another embodiment, the pharmaceutical composition provided herein comprises (i) from about 6 to about 9% by weight of the compound provided herein (e.g., compound A1 or A2), or an isotopic variant thereof, or a pharmaceutically acceptable salt thereof, or a pharmaceutically acceptable solvate thereof; and (ii) from about 18 to about 25% by weight of a dispersant or carrier, about 10% by weight of a disintegrant, about 65% by weight of a filler, about 1% by weight of a glidant, and about 0.3% by weight of a lubricant.

In one embodiment, the pharmaceutical composition provided herein comprises (i) from about 5 to about 25% of compound A1; and (ii) from about 10 to about 25% by weight of a polyvinyl pyrrolidone (e.g., PVP-K30), from about 5 to about 25% by weight of a polyvinyl pyrrolidone (e.g., POLYPLASDONE® XL), from about 25 to about 80% by weight of a microcrystalline cellulose (e.g., AVICEL® PH 102), from about 0.1 to about 5% by weight of a colloidal silicon dioxide (e.g., CAB-O-SIL® M-5P), and from about 0.05 to about 2% by weight of magnesium stearate.

In another embodiment, the pharmaceutical composition provided herein comprises (i) from about 5 to about 10% of compound A1; and (ii) from about 15 to about 25% by weight of a polyvinyl pyrrolidone (e.g., PVP-K30), from about 5 to about 15% by weight of a polyvinyl pyrrolidone (e.g., POLYPLASDONE® XL), from about 30 to about 70% by weight of a microcrystalline cellulose (e.g., AVICEL® PH 102), from about 0.5 to about 1.5% by weight of a colloidal silicon dioxide (e.g., CAB-O-SIL® M-5P), and from about 0.1 to about 1% by weight of magnesium stearate.

In yet another embodiment, the pharmaceutical composition provided herein comprises (i) from about 6 to about 9% by weight of compound A1; and (ii) from about 18 to about 25% by weight of a polyvinyl pyrrolidone (e.g., PVP-K30), from about 6 to about 10% by weight of a polyvinyl pyrrolidone (e.g., POLYPLASDONE® XL), from about 30 to about 70% by weight of a microcrystalline cellulose (e.g., AVICEL® PH 102), from about 0.5 to about 1.5% by weight of a colloidal silicon dioxide (e.g., CAB-O-SIL® M-5P), and from about 0.3 to about 0.7% by weight of magnesium stearate.

In still another embodiment, the pharmaceutical composition provided herein comprises (i) from about 6 to about 9% by weight of compound A1; and (ii) from about 18 to about 25% by weight of a polyvinyl pyrrolidone (e.g., PVP-K30), about 10% by weight of a polyvinyl pyrrolidone (e.g., POLYPLASDONE® XL), about 65% by weight of a microcrystalline cellulose (e.g., AVICEL® PH 102), about 1% by weight of a colloidal silicon dioxide (e.g., CAB-O-SIL® M-5P), and about 0.3% by weight of magnesium stearate.

In one embodiment, provided herein is a pharmaceutical composition comprising spray-dried particles provided herein; and a second pharmaceutically acceptable excipient. In one embodiment, the pharmaceutical composition provided herein is in a solid form. In another embodiment, the pharmaceutical composition provided herein is a tablet. In yet another embodiment, the pharmaceutical composition provided herein is a capsule.

In certain embodiments, the pharmaceutical composition provided herein comprises from about 10 to about 90%, from about 10 to about 50%, from about 20 to about 50%, or from about 25 to about 35% by weight of spray-dried particles provided herein. In certain embodiments, the pharmaceutical composition provided herein comprises about 20%, about 21%, about 22%, about 23%, about 24%, about 25%, about 26%, about 27%, about 28%, about 29%, about 30%, about 31%, about 32%, about 33%, about 34%, or about 35% by weight of spray dried particles provided herein. In certain embodiments, the pharmaceutical composition provided herein comprises from about 25 to about 35% by weight of spray dried particles provided herein.

In certain embodiments, the pharmaceutical composition provided herein comprises from about 10 to about 90%, from about 50 to about 90%, from about 50 to about 80%, or from about 60 to about 75% by weight of a second excipient. In certain embodiments, the pharmaceutical composition provided herein comprises about 60%, about 62%, about 64%, about 65%, about 66%, about 68%, about 70%, about 72%, about 74%, about 75%, about 76%, about 78%, or about 80% by weight of a second excipient. In certain embodiments, the pharmaceutical composition provided herein comprises about 65% by weight of a second excipient. In certain embodiments, the pharmaceutical composition provided herein comprises about 75% by weight of a second excipient.

In one embodiment, the pharmaceutical composition provided herein comprises from about 10 to about 50% by weight of spray-dried particles provided herein and from about 50 to about 90% by weight of a second excipient.

In another embodiment, the pharmaceutical composition provided herein comprises from about 20 to about 50% by weight of spray-dried particles provided herein and from about 50 to about 80% by weight of a second excipient.

In yet another embodiment, the pharmaceutical composition provided herein comprises from about 25 to about 35% by weight of spray-dried particles provided herein and from about 65 to about 75% by weight of a second excipient.

In one embodiment, provided herein is a pharmaceutical composition comprising (i) spray-dried particles provided herein; and (ii) a disintegrant, a filler, a glidant, a lubricant, an organic acid, a surfactant, or a mixture thereof.

In another embodiment, provided herein is a pharmaceutical composition comprising (i) spray-dried particles provided herein; and (ii) a disintegrant, a filler, a glidant, and a lubricant.

In yet another embodiment, provided herein is a pharmaceutical composition comprising (i) spray-dried particles provided herein; and (ii) a disintegrant, a filler, a glidant, a lubricant, and an organic acid.

In yet another embodiment, provided herein is a pharmaceutical composition comprising (i) spray-dried particles provided herein; and (ii) a disintegrant, a filler, a glidant, a lubricant, and a surfactant.

In still another embodiment, provided herein is a pharmaceutical composition comprising (i) spray-dried particles provided herein; and (ii) a disintegrant, a filler, a glidant, a lubricant, an organic acid, and a surfactant.

In certain embodiments, the pharmaceutical composition provided herein comprises from about 1 to about 50%, from about 2 to about 20%, or from about 5 to about 15% by weight of a disintegrant. In certain embodiments, the pharmaceutical composition provided herein comprises about 5%, about 6%, about 7%, about 8%, about 9%, about 10%, about 11%, about 12%, about 13%, about 14%, or about 15% by weight of a disintegrant. In certain embodiments, the pharmaceutical composition provided herein comprises about 6% by weight of a disintegrant. In certain embodiments, the pharmaceutical composition provided herein comprises about 10% by weight of a disintegrant. In certain embodiments, the disintegrant is a crosslinked polyvinyl pyrrolidone, croscarmellose sodium, or a mixture thereof. In certain embodiments, the disintegrant is a crosslinked polyvinyl pyrrolidone. In certain embodiments, the crosslinked polyvinyl pyrrolidone is POLYPLASDONE® XL. In certain embodiments, the disintegrant is croscarmellose sodium. In certain embodiments, the crosslinked polyvinyl pyrrolidone is AC-DI-SOL®.

In certain embodiments, the pharmaceutical composition provided herein comprises from about 10 to about 95%, from about 20 to about 90%, from about 25 to about 75%, or from about 30 to about 70% by weight of a filler. In certain embodiments, the pharmaceutical composition provided herein comprises about 25%, about 30%, about 35%, about 40%, about 45%, about 50%, about 55%, about 60%, about 65%, about 70%, or about 75% by weight of a filler. In certain embodiments, the pharmaceutical composition provided herein comprises about 30% by weight of a filler. In certain embodiments, the pharmaceutical composition provided herein comprises about 50% by weight of a filler. In certain embodiments, the pharmaceutical composition provided herein comprises about 65% by weight of a filler. In certain embodiments, the filler is a microcrystalline cellulose, lactose, or a mixture thereof. In certain embodiments, the filler is a microcrystalline cellulose. In certain embodiments, the filler is AVICEL® PH 102. In certain embodiments, the filler is lactose. In certain embodiments, the filler is lactose FAST FLO® 316.

In certain embodiments, the pharmaceutical composition provided herein comprises from about 0.1 to about 5%, from about 0.1 to about 2%, from about 0.2 to about 1.5%, or from about 0.5 to about 1% by weight of a glidant. In certain embodiments, the pharmaceutical composition provided herein comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% by weight of a glidant. In certain embodiments, the pharmaceutical composition provided herein comprises from about 0.5 to about 1.5% by weight of a glidant. In certain embodiments, the glidant is a colloidal silicon dioxide. In certain embodiment, the glidant is CAB-O-SIL®.

In certain embodiments, the pharmaceutical composition provided herein comprises from about 0.05 to about 5%, from about 0.1 to about 5%, from about 0.1 to about 2%, or from about 0.2 to about 1% by weight of a lubricant. In certain embodiments, the pharmaceutical composition provided herein comprises about 0.1%, about 0.2%, about 0.3%, about 0.4%, about 0.5%, about 0.6%, about 0.7%, about 0.8%, about 0.9%, or about 1% by weight of a lubricant. In certain embodiments, the pharmaceutical composition provided herein comprises from about 0.2 to about 0.7% by weight of a lubricant. In certain embodiments, the pharmaceutical composition provided herein comprises about 0.5% by weight of a lubricant. In certain embodiments, the lubricant is magnesium stearate.

In certain embodiments, the pharmaceutical composition provided herein comprises from about 1 to about 25%, from about 2 to about 20%, from about 5 to about 15%, or from about 10 to about 15% by weight of an organic acid. In certain embodiments, the pharmaceutical composition provided herein comprises about 5%, about 6%, about 7%, about 8%, about 9%, about 10%, about 11%, about 12%, about 13%, about 14%, or about 15% by weight of an organic acid. In certain embodiments, the pharmaceutical composition provided herein comprises from about 10 to about 15% by weight of an organic acid. In certain embodiments, the organic acid is tartaric acid.

In certain embodiments, the pharmaceutical composition provided herein comprises from about 1 to about 30%, from about 2 to about 25%, or from about 5 to about 25% by weight of a surfactant. In certain embodiments, the pharmaceutical composition provided herein comprises about 10%, about 11%, about 12%, about 13%, about 14%, about 15%, about 16%, about 17%, about 18%, about 19%, or about 20% by weight of a surfactant. In certain embodiments, the pharmaceutical composition provided herein comprises from about 10 to about 20% by weight of a surfactant. In certain embodiments, the surfactant is sodium lauryl sulfate.

In one embodiment, the pharmaceutical composition provided herein comprises (i) from about 10 to about 50% of spray-dried particles provided herein; and (ii) from about 5 to about 25% by weight of a disintegrant, from about 25 to about 80% by weight of a filler, from about 0.1 to about 5% by weight of a glidant, and from about 0.05 to about 2% by weight of a lubricant.

In another embodiment, the pharmaceutical composition provided herein comprises (i) from about 20 to about 40% of spray-dried particles provided herein; and (ii) from about 5 to about 15% by weight of a disintegrant, from about 30 to about 70% by weight of a filler, from about 0.5 to about 1.5% by weight of a glidant, and from about 0.1 to about 1% by weight of a lubricant.

In yet another embodiment, the pharmaceutical composition provided herein comprises (i) from about 25 to about 35% of spray-dried particles provided herein; and (ii) from about 6 to about 10% by weight of a disintegrant, from about 30 to about 70% by weight of a filler, from about 0.5 to about 1.5% by weight of a glidant, and from about 0.3 to about 0.7% by weight of a lubricant.

In still another embodiment, the pharmaceutical composition provided herein comprises (i) from about 25 to about 35% by weight of spray-dried particles provided herein; and (ii) about 10% by weight of a disintegrant, about 65% by weight of a filler, about 1% by weight of a glidant, and about 0.3% by weight of a lubricant.

In one embodiment, the pharmaceutical composition provided herein comprises (i) from about 10 to about 50% of spray-dried particles provided herein; and (ii) from about 5 to about 25% by weight of a polyvinyl pyrrolidone (e.g., POLYPLASDONE® XL), from about 25 to about 80% by weight of a microcrystalline cellulose (e.g., AVICEL® PH 102), from about 0.1 to about 5% by weight of a colloidal silicon dioxide (e.g., CAB-O-SIL® M-5P), and from about 0.05 to about 2% by weight of magnesium stearate.

In another embodiment, the pharmaceutical composition provided herein comprises (i) from about 20 to about 40% of spray-dried particles provided herein; and (ii) from about 5 to about 15% by weight of a polyvinyl pyrrolidone (e.g., POLYPLASDONE® XL), from about 30 to about 70% by weight of a microcrystalline cellulose (e.g., AVICEL® PH 102), from about 0.5 to about 1.5% by weight of a colloidal silicon dioxide (e.g., CAB-O-SIL® M-5P), and from about 0.1 to about 1% by weight of magnesium stearate.

In yet another embodiment, the pharmaceutical composition provided herein comprises (i) from about 25 to about 35% of spray-dried particles provided herein; and (ii) from about 6 to about 10% by weight of a polyvinyl pyrrolidone (e.g., POLYPLASDONE® XL), from about 30 to about 70% by weight of a microcrystalline cellulose (e.g., AVICEL® PH 102), from about 0.5 to about 1.5% by weight of a colloidal silicon dioxide (e.g., CAB-O-SIL® M-5P), and from about 0.3 to about 0.7% by weight of magnesium stearate.

In still another embodiment, the pharmaceutical composition provided herein comprises (i) from about 25 to about 35% of spray-dried particles provided herein; and (ii) about 10% by weight of a polyvinyl pyrrolidone (e.g., POLYPLASDONE® XL), about 65% by weight of a microcrystalline cellulose (e.g., AVICEL® PH 102), about 1% by weight of a colloidal silicon dioxide (e.g., CAB-O-SIL® M-5P), and about 0.3% by weight of magnesium stearate.

In one embodiment, provided herein is a granular pharmaceutical composition comprising the compound provided herein (e.g., compound A1 or A2), or an isotopic variant thereof, or a pharmaceutically acceptable salt thereof, or a pharmaceutically acceptable solvate thereof; and a pharmaceutically acceptable excipient (“granular excipient”).

In certain embodiments, the granular composition provided herein comprises from about 1 to about 50%, from about 2 to about 50%, or from about 5 to about 25% by weight of the compound provided herein (e.g., compound A1 or A2), or an isotopic variant thereof, or a pharmaceutically acceptable salt thereof, or a pharmaceutically acceptable solvate thereof. In certain embodiments, the granular composition provided herein comprises about 5%, about 10%, about 15%, about 20%, about 25%, or about 30% by weight of the compound provided herein (e.g., compound A1 or A2), or an isotopic variant thereof, or a pharmaceutically acceptable salt thereof, or a pharmaceutically acceptable solvate thereof. In certain embodiments, the granular composition provided herein comprises about 5%, about 6%, about 7%, about 8%, about 9%, or about 10% by weight of the compound provided herein (e.g., compound A1 or A2), or an isotopic variant thereof, or a pharmaceutically acceptable salt thereof, or a pharmaceutically acceptable solvate thereof. In certain embodiments, the granular composition provided herein comprises about 20%, about 21%, about 22%, about 23%, about 24%, about 25%, about 26%, about 27%, about 28%, about 29%, about 30%, about 31%, about 32%, about 33%, about 34%, or about 31% by weight of the compound provided herein (e.g., compound A1 or A2), or an isotopic variant thereof, or a pharmaceutically acceptable salt thereof, or a pharmaceutically acceptable solvate thereof. In certain embodiments, the granular composition provided herein comprises about 25% by weight of the compound provided herein (e.g., compound A1 or A2), or an isotopic variant thereof, or a pharmaceutically acceptable salt thereof, or a pharmaceutically acceptable solvate thereof. In certain embodiments, the granular composition provided herein comprises about 6% by weight of the compound provided herein (e.g., compound A1 or A2), or an isotopic variant thereof, or a pharmaceutically acceptable salt thereof, or a pharmaceutically acceptable solvate thereof.

In certain embodiments, the granular composition provided herein comprises from about 50 to about 99%, from about 50 to about 98%, or from about 75 to about 95% by weight of a granular excipient. In certain embodiments, the granular composition provided herein comprises about 70%, about 71%, about 72%, about 73, about 74%, about 75%, about 76%, about 77%, about 78%, about 79%, or about 80% by weight of a granular excipient. In certain embodiments, the granular composition provided herein comprises about 90%, about 91%, about 92%, about 93%, about 94%, or about 95% by weight of a granular excipient. In certain embodiments, the granular composition provided herein comprises about 75% by weight of a granular excipient. In certain embodiments, the granular composition provided herein comprises about 94% by weight of a granular excipient.

In one embodiment, the granular composition provided herein comprises from about 5 to about 40% by weight of the compound provided herein (e.g., compound A1 or A2), or an isotopic variant thereof, or a pharmaceutically acceptable salt thereof, or a pharmaceutically acceptable solvate thereof; and from about 60 to about 95% by weight of a granular excipient.

In another embodiment, the granular composition provided herein comprises from about 5 to about 25% by weight of the compound provided herein (e.g., compound A1 or A2), or an isotopic variant thereof, or a pharmaceutically acceptable salt thereof, or a pharmaceutically acceptable solvate thereof; and from about 75 to about 95% by weight of a granular excipient.

In yet another embodiment, the granular composition provided herein comprises about 25% by weight of the compound provided herein (e.g., compound A1 or A2), or an isotopic variant thereof, or a pharmaceutically acceptable salt thereof, or a pharmaceutically acceptable solvate thereof; and from about 75% by weight of a granular excipient.

In one embodiment, provided herein is a granular composition comprising the compound provided herein (e.g., compound A1 or A2), or an isotopic variant thereof, or a pharmaceutically acceptable salt thereof, or a pharmaceutically acceptable solvate thereof; and a dispersant or carrier, a disintegrant, a filler, a glidant, a lubricant, or a mixture thereof.

In another embodiment, provided herein is a granular composition comprising the compound provided herein (e.g., compound A1 or A2), or an isotopic variant thereof, or a pharmaceutically acceptable salt thereof, or a pharmaceutically acceptable solvate thereof; and a dispersant or carrier, a glidant, and a lubricant.

In yet another embodiment, provided herein is a granular composition comprising the compound provided herein (e.g., compound A1 or A2), or an isotopic variant thereof, or a pharmaceutically acceptable salt thereof, or a pharmaceutically acceptable solvate thereof; and a dispersant or carrier, a disintegrant, a filler, a glidant, and a lubricant.

In certain embodiments, the granular composition provided herein comprises from about 10 to about 90%, from about 15 to about 80%, or from about 15 to about 75% by weight of a dispersant or carrier. In certain embodiments, the granular composition provided herein comprises about 5%, about 6%, about 7%, about 8%, about 9%, or about 10% by weight of a dispersant or carrier. In certain embodiments, the granular composition provided herein comprises about 70%, about 71%, about 72%, about 73%, about 74%, about 75%, about 76%, about 77%, about 78%, about 79%, or about 80% by weight of a dispersant or carrier. In certain embodiments, the granular composition provided herein comprises about 75% by weight of a dispersant or carrier.

In certain embodiments, the granular dispersant or carrier is a hypromellose, a hypromellose acetate succinate, a hypromellose phthalate, a methacrylic acid and ethyl acrylate copolymer, a poloxamer, a polyethylene glycol, a povidone, a tocopherol polyethylene glycol succinate, or a mixture thereof. In certain embodiments, the granular dispersant or carrier is a povidone. In certain embodiments, the granular dispersant or carrier is a povidone having an average molecular weight of about 40,000 Da. In certain embodiments, the granular dispersant or carrier is PVP-K30. In certain embodiments, the granular dispersant or carrier is a hypromellose acetate succinate. In certain embodiments, the granular dispersant or carrier is a hypromellose acetate succinate having an average molecular weight of about 18,000 Da. In certain embodiments, the granular dispersant or carrier is HPMCAS, MF grade.

In certain embodiments, the granular composition provided herein comprises from about 1 to about 50%, from about 2 to about 20%, from about 5 to about 15%, or from about 5 to about 10% by weight of a disintegrant. In certain embodiments, the granular composition provided herein comprises about 5%, about 6%, about 7%, about 8%, about 9%, about 10%, about 11%, about 12%, about 13%, about 14%, or about 15% by weight of a disintegrant. In certain embodiments, the granular composition provided herein comprises about 6% by weight of a disintegrant. In certain embodiments, the granular disintegrant is a crosslinked polyvinyl pyrrolidone, croscarmellose sodium, or a mixture thereof. In certain embodiments, the granular disintegrant is a crosslinked polyvinyl pyrrolidone. In certain embodiments, the granular disintegrant is POLYPLASDONE® XL. In certain embodiments, the granular disintegrant is croscarmellose sodium. In certain embodiments, the granular disintegrant is AC-DI-SOL®.

In certain embodiments, the granular composition provided herein comprises from about 10 to about 95%, from about 20 to about 90%, from about 25 to about 75%, or from about 30 to about 70% by weight of a filler. In certain embodiments, the granular composition provided herein comprises about 25%, about 30%, about 35%, about 40%, about 45%, about 50%, about 55%, about 60%, about 65%, about 70%, or about 75% by weight of a filler. In certain embodiments, the granular composition provided herein comprises about 30%, about 50%, about 65% by weight of a filler. In certain embodiments, the granular filler is a microcrystalline cellulose, lactose, or a mixture thereof. In certain embodiments, the granular filler is a microcrystalline cellulose. In certain embodiments, the granular filler is AVICEL® PH 102. In certain embodiments, the granular filler is lactose. In certain embodiments, the granular filler is lactose FAST FLO® 316. In certain embodiments, the granular filler is a mixture of microcrystalline cellulose and lactose. In certain embodiments, the granular filler is a mixture of AVICEL® PH 102 and lactose FAST FLO® 316.

In certain embodiments, the granular composition provided herein comprises from about 0.1 to about 5%, from about 0.5 to about 5%, from about 1 to about 3%, or from about 1 to about 2.5% by weight of a glidant. In certain embodiments, the granular composition provided herein comprises about 0.5%, about 1%, about 1.5%, about 2%, about 2.5%, or about 3% by weight of a glidant. In certain embodiments, the granular composition provided herein comprises about 2% by weight of a glidant. In certain embodiments, the granular glidant is a colloidal silicon dioxide. In certain embodiment, the granular glidant is CAB-O-SIL®.

In certain embodiments, the granular composition provided herein comprises from about 0.01 to about 2%, from about 0.05 to about 0.1%, from about 0.2 to about 0.5%, or from about 0.2 to about 0.4% by weight of a lubricant. In certain embodiments, the granular composition provided herein comprises about 0.1%, about 0.15%, about 0.2%, about 0.25%, about 0.3%, about 0.35%, about 0.4%, about 0.45%, or about 0.5% by weight of a glidant. In certain embodiments, the granular composition provided herein comprises from about 0.2 to about 0.4% by weight of a lubricant. In certain embodiments, the granular lubricant is magnesium stearate.

In one embodiment, the granular composition provided herein comprises from about 5 to about 40% by weight of the compound provided herein (e.g., compound A1 or A2), or an isotopic variant thereof, or a pharmaceutically acceptable salt thereof, or a pharmaceutically acceptable solvate thereof; and from about 5 to about 80% by weight of a dispersant or carrier, from about 0.1 to about 5% by weight of a glidant, and from about 0.1 to about 5% by weight of a lubricant.

In another embodiment, the granular composition provided herein comprises from about 5 to about 25% by weight of the compound provided herein (e.g., compound A1 or A2), or an isotopic variant thereof, or a pharmaceutically acceptable salt thereof, or a pharmaceutically acceptable solvate thereof; and from about 50 to about 80% by weight of a dispersant or carrier, from about 1 to about 2.5% by weight of a glidant, and from about 0.2 to about 0.5% by weight of a lubricant.

In yet another embodiment, the granular composition provided herein comprises about 25% by weight of the compound provided herein (e.g., compound A1 or A2), or an isotopic variant thereof, or a pharmaceutically acceptable salt thereof, or a pharmaceutically acceptable solvate thereof; and about 75% by weight of a dispersant or carrier, about 2% by weight of a glidant, and from about 0.25 to about 0.4% by weight of a lubricant.

In one embodiment, the granular composition provided herein comprises from about 5 to about 40% by weight of compound A1; and from about 5 to about 80% by weight of a polyvinyl pyrrolidone (e.g., PVP-K30), from about 0.1 to about 5% by weight of a colloidal silicon dioxide (e.g., CAB-O-SIL® M-5P), and from about 0.1 to about 5% by weight of magnesium stearate.

In another embodiment, the granular composition provided herein comprises from about 5 to about 25% by weight compound A1; and from about 50 to about 80% by weight of a polyvinyl pyrrolidone (e.g., PVP-K30), from about 1 to about 2.5% by weight of a colloidal silicon dioxide (e.g., CAB-O-SIL® M-5P), and from about 0.2 to about 0.5% by weight of magnesium stearate.

In yet another embodiment, the granular composition provided herein comprises about 25% by weight of compound A1; and about 75% by weight of a polyvinyl pyrrolidone (e.g., PVP-K30), about 2% by weight of a colloidal silicon dioxide (e.g., CAB-O-SIL® M-5P), and from about 0.25 to about 0.4% by weight of magnesium stearate.

In one embodiment, provided herein is a granular pharmaceutical composition comprising spray-dried particles provided herein; and a second pharmaceutically acceptable excipient (“second granular excipient”).

In certain embodiments, the granular composition provided herein comprises from about 10 to about 99%, from about 20 to about 99%, or from about 25 to about 99% by weight of spray-dried particles provided herein. In certain embodiments, the granular composition provided herein comprises about 20%, about 25%, about 30%, about 35%, about 40%, about 45%, or about 50% by weight of spray-dried particles provided herein. In certain embodiments, the granular composition provided herein comprises about 95%, about 96%, about 97%, about 98%, or about 99% by weight of spray-dried particles provided herein. In certain embodiments, the granular composition provided herein comprises about 25% by weight of spray-dried particles provided herein. In certain embodiments, the granular composition provided herein comprises about 98% by weight of spray-dried particles provided herein.

In one embodiment, provided herein is a granular pharmaceutical composition comprising spray-dried particles provided herein; and a pharmaceutically acceptable excipient (“extra-SDD excipient”), which is not part of the spray-dried particles.

In certain embodiments, the granular composition provided herein comprises from about 5 to about 99%, from about 10 to about 99%, or from about 25 to about 99% by weight of spray-dried particles provided herein. In certain embodiments, the granular composition provided herein comprises about 21%, about 22%, about 23%, about 24%, about 25%, about 26%, about 27%, about 28%, about 29%, or about 30% by weight of spray-dried particles provided herein. In certain embodiments, the granular composition provided herein comprises about 95%, about 96%, about 97%, about 98%, or about 99% by weight of spray-dried particles provided herein. In certain embodiments, the granular composition provided herein comprises about 98% by weight of spray-dried particles provided herein.

In certain embodiments, the granular composition provided herein comprises from about 0.5 to about 80%, from about 1 to about 80%, or from about 2 to about 80% by weight of an extra-SDD excipient. In certain embodiments, the granular composition provided herein comprises about 70%, about 71%, about 72%, about 73%, about 74%, about 75%, about 76%, about 77%, about 78%, about 79%, or about 80% by weight of an extra-SDD excipient. In certain embodiments, the granular composition provided herein comprises about 0.5%, about 1%, about 1.5%, about 2%, about 2.5%, about 3%, about 3.5%, about 4%, about 4.5%, or about 5% by weight of an extra-SDD excipient. In certain embodiments, the granular composition provided herein comprises from about 2 to about 2.5% by weight of an extra-SDD excipient.

In one embodiment, the granular composition provided herein comprises from about 20 to about 99% by weight of spray-dried particles provided herein; and from about 1 to about 80% by weight of an extra-SDD excipient.

In another embodiment, the granular composition provided herein comprises from about 25 to about 99% by weight of spray-dried particles provided herein; and from about 1 to about 75% by weight of an extra-SDD excipient.

In yet another embodiment, the granular composition provided herein comprises about 98% by weight of spray-dried particles provided herein and from about 2 to about 2.5% by weight of an extra-SDD excipient.

In one embodiment, provided herein is a granular composition comprising spray-dried particles provided herein; and an extra-SDD disintegrant, an extra-SDD filler, an extra-SDD glidant, an extra-SDD lubricant, or a mixture thereof.

In another embodiment, provided herein is a granular composition comprising spray-dried particles provided herein; and an extra-SDD glidant and an extra-SDD lubricant.

In yet another embodiment, provided herein is a granular composition comprising spray-dried particles provided herein; and an extra-SDD disintegrant, an extra-SDD filler, an extra-SDD glidant, and an extra-SDD lubricant.

In certain embodiments, the granular composition provided herein comprises from about 1 to about 50%, from about 2 to about 20%, from about 5 to about 15%, or from about 5 to about 10% by weight of an extra-SDD disintegrant. In certain embodiments, the granular composition provided herein comprises about 5%, about 6%, about 7%, about 8%, about 9%, about 10%, about 11%, about 12%, about 13%, about 14%, or about 15% by weight of an extra-SDD disintegrant. In certain embodiments, the granular composition provided herein comprises about 6% by weight of an extra-SDD disintegrant. In certain embodiments, the extra-SDD disintegrant is a crosslinked polyvinyl pyrrolidone or croscarmellose sodium. In certain embodiments, the extra-SDD disintegrant is a crosslinked polyvinyl pyrrolidone. In certain embodiments, the extra-SDD disintegrant is POLYPLASDONE® XL. In certain embodiments, the extra-SDD disintegrant is croscarmellose sodium. In certain embodiments, the extra-SDD disintegrant is AC-DI-SOL®.

In certain embodiments, the granular composition provided herein comprises from about 10 to about 95%, from about 20 to about 90%, from about 25 to about 75%, or from about 30 to about 70% by weight of an extra-SDD filler. In certain embodiments, the granular composition provided herein comprises about 25%, about 30%, about 35%, about 40%, about 45%, about 50%, about 55%, about 60%, about 65%, about 70%, or about 75% by weight of an extra-SDD filler. In certain embodiments, the granular composition provided herein comprises about 30%, about 50%, about 65% by weight of an extra-SDD filler. In certain embodiments, the extra-SDD filler is a microcrystalline cellulose, lactose, or a mixture thereof. In certain embodiments, the extra-SDD filler is a microcrystalline cellulose. In certain embodiments, the extra-SDD filler is AVICEL® PH 102. In certain embodiments, the extra-SDD filler is lactose. In certain embodiments, the extra-SDD filler is lactose FAST FLO® 316. In certain embodiments, the extra-SDD filler is a mixture of microcrystalline cellulose and lactose.

In certain embodiments, the granular composition provided herein comprises from about 0.1 to about 5%, from about 0.5 to about 5%, from about 1 to about 3%, or from about 1 to about 2.5% by weight of an extra-SDD glidant. In certain embodiments, the granular composition provided herein comprises about 0.5%, about 1%, about 1.5%, about 2%, about 2.5%, or about 3% by weight of an extra-SDD glidant. In certain embodiments, the granular composition provided herein comprises from about 2 to about 2.5% by weight of an extra-SDD glidant. In certain embodiments, the extra-SDD glidant is a colloidal silicon dioxide. In certain embodiment, the extra-SDD glidant is CAB-O-SIL®.

In certain embodiments, the granular composition provided herein comprises from about 0.01 to about 2%, from about 0.05 to about 0.1%, from about 0.2 to about 0.5%, or from about 0.2 to about 0.4% by weight of an extra-SDD lubricant. In certain embodiments, the granular composition provided herein comprises about 0.1%, about 0.15%, about 0.2%, about 0.25%, about 0.3%, about 0.35%, about 0.4%, about 0.45%, or about 0.5% by weight of an extra-SDD lubricant. In certain embodiments, the granular composition provided herein comprises from about 0.2 to about 0.4% by weight of an extra-SDD lubricant. In certain embodiments, the extra-SDD lubricant is magnesium stearate.

In one embodiment, the granular composition provided herein comprises from about 5 to about 99% by weight of spray-dried particles provided herein; and from about 0.1 to about 5% by weight of an extra-SDD glidant, and from about 0.1 to about 5% by weight of an extra-SDD lubricant.

In another embodiment, the granular composition provided herein comprises from about 20 to about 99% by weight of spray-dried particles provided herein; and from about 1 to about 2.5% by weight of an extra-SDD glidant, and from about 0.2 to about 0.5% by weight of an extra-SDD lubricant.

In yet another embodiment, the granular composition provided herein comprises about 98% by weight of spray-dried particles provided herein; and from about 2 to about 2.5% by weight of an extra-SDD glidant, and from about 0.25 to about 0.4% by weight of an extra-SDD lubricant.

In one embodiment, the granular composition provided herein comprises from about 5 to about 99% by weight of spray-dried particles provided herein; and from about 0.1 to about 5% by weight of an extra-SDD colloidal silicon dioxide (e.g., CAB-O-SIL® M-5P), and from about 0.1 to about 5% by weight of extra-SDD magnesium stearate.

In another embodiment, the granular composition provided herein comprises from about 20 to about 99% by weight of spray-dried particles provided herein; and from about 1 to about 2.5% by weight of an extra-SDD colloidal silicon dioxide (e.g., CAB-O-SIL® M-5P), and from about 0.2 to about 0.5% by weight of extra-SDD magnesium stearate.

In yet another embodiment, the granular composition provided herein comprises about 98% by weight of spray-dried particles provided herein; and from about 2 to about 2.5% by weight of an extra-SDD colloidal silicon dioxide (e.g., CAB-O-SIL® M-5P), and from about 0.25 to about 0.4% by weight of extra-SDD magnesium stearate.

In certain embodiments, the granular composition provided herein has an average particle size ranging from about 100 to about 2,000 μm, from about 200 to about 1,000 μm, from about 200 to about 750 μm, or from about 400 to about 600 μm. In certain embodiments, the granular composition provided herein have an average particle size ranging from about 400 to about 600 μm. In certain embodiments, the particle size distribution of the granular composition provided herein is determined according to Method 786 in USP XXVI (2003).

In certain embodiments, the granular composition provided herein has a bulk density ranging from about 0.1 to about 1 g/mL, from about 0.2 to about 0.8 g/mL, from about 0.3 to about 0.7 g/mL, or from about 0.3 to about 0.6 g/mL. In certain embodiments, the granular composition has a bulk density of about 0.1, about 0.15, about 0.2, about 0.25, about 0.3, about 0.35, about 0.4, about 0.45, or about 0.5 g/mL. In certain embodiments, the granular composition has a bulk density of about 0.4 g/mL. In certain embodiments, the bulk density of the granular composition provided herein is quantitated according to Method 616 in USP XXVI (2003).

In certain embodiments, the granular composition provided herein has a tapped density ranging from about 0.1 to about 1 g/mL, from about 0.2 to about 0.8 g/mL, from about 0.3 to about 0.7 g/mL, or from about 0.4 to about 0.6 g/mL. In certain embodiments, the granular composition has a tapped density of about 0.2, about 0.25, about 0.3, about 0.35, about 0.4, about 0.45, about 0.5, about 0.55, or about 0.6 g/mL. In certain embodiments, the granular composition has a tapped density of about 0.5 g/mL. In certain embodiments, the tapped density of the granular composition provided herein is quantitated according to Method 616 in USP XXVI (2003).

In certain embodiments, the granular composition provided herein has a Carr index ranging from about 5 to about 50, from about 10 to about 50, from about 20 to about 40, or from about 20 to about 30. In certain embodiments, the granular composition has a Carr index of about 20, about 21, about 22, about 23, about 24, about 25, about 26, about 27, about 28, about 29, about 30, about 31, about 32, about 33, about 34, or about 35. In certain embodiments, the granular composition has a Carr index of about 25.

In one embodiment, provided herein is a pharmaceutical composition comprising (i) intragranular components comprising: the compound provided herein (e.g., compound A1 or A2), or an isotopic variant thereof, or a pharmaceutically acceptable salt thereof, or a pharmaceutically acceptable solvate thereof; and a pharmaceutically acceptable excipient; and (ii) extragranular components comprising: a pharmaceutically acceptable excipient.

In certain embodiments, the pharmaceutical composition provided herein comprises from about 1 to about 50%, from about 2 to about 25%, from about 5 to about 20%, from about 5 to about 15%, or from about 5 to about 10% by weight of the compound provided herein (e.g., compound A1 or A2), or an isotopic variant thereof, or a pharmaceutically acceptable salt thereof, or a pharmaceutically acceptable solvate thereof. In certain embodiments, the pharmaceutical composition provided herein comprises about 5%, about 5.5%, about 6%, about 6.5%, about 7%, about 7.5%, about 8%, about 8.5%, about 9%, about 9.5%, or about 10% by weight of the compound provided herein (e.g., compound A1 or A2), or an isotopic variant thereof, or a pharmaceutically acceptable salt thereof, or a pharmaceutically acceptable solvate thereof. In certain embodiments, the pharmaceutical composition comprises from about 6 to about 9% by weight of the compound provided herein (e.g., compound A1 or A2), or an isotopic variant thereof, or a pharmaceutically acceptable salt thereof, or a pharmaceutically acceptable solvate thereof. In certain embodiments, the pharmaceutical composition comprises from about 6 to about 6.5% by weight of the compound provided herein (e.g., compound A1 or A2), or an isotopic variant thereof, or a pharmaceutically acceptable salt thereof, or a pharmaceutically acceptable solvate thereof. In certain embodiments, the pharmaceutical composition comprises from about 8 to about 8.5% by weight of the compound provided herein (e.g., compound A1 or A2), or an isotopic variant thereof, or a pharmaceutically acceptable salt thereof, or a pharmaceutically acceptable solvate thereof.

In certain embodiments, the pharmaceutical composition provided herein comprises from about 5 to about 99.5%, from 80 to about 99.5%, or from about 90 to about 99.5% by weight of an intragranular excipient. In certain embodiments, the pharmaceutical composition provided herein comprises about 95%, about 96%, about 97%, about 98%, about 99%, or about 99.5% by weight of an intragranular excipient.

In certain embodiments, the pharmaceutical composition provided herein comprises from about 5 to about 50%, from about 10 to about 50%, from about 15 to about 30%, or from about 15 to about 25% by weight of an intragranular excipient. In certain embodiments, the pharmaceutical composition provided herein comprises about 15%, about 16%, about 17%, about 18%, about 19%, about 20%, about 21%, about 22%, about 23%, about 24%, about 25%, about 26%, about 27%, about 28%, about 29%, or about 30% by weight of an intragranular excipient.

In certain embodiments, the pharmaceutical composition provided herein comprises from about 0.1 to about 5%, from about 0.1 to about 2%, from about 0.2 to about 1.5%, or from about 0.5 to about 1% by weight of an extragranular excipient. In certain embodiments, the pharmaceutical composition provided herein comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% by weight of an extragranular excipient.

In certain embodiments, the pharmaceutical composition provided herein comprises from about 10 to about 90%, from about 50 to about 90%, from about 50 to about 80%, or from about 60 to about 75% by weight of an extragranular excipient. In certain embodiments, the pharmaceutical composition provided herein comprises about 60%, about 62%, about 64%, about 65%, about 66%, about 68%, about 70%, about 72%, about 74%, about 75%, about 76%, about 78%, or about 80% by weight of an extragranular excipient. In certain embodiments, the pharmaceutical composition provided herein comprises about 65% by weight of an extragranular excipient. In certain embodiments, the pharmaceutical composition provided herein comprises about 75% by weight of an extragranular excipient.

In one embodiment, provided herein is a pharmaceutical composition comprising (i) intragranular components comprising: from about 5 to about 20% by weight of the compound provided herein (e.g., compound A1 or A2), or an isotopic variant thereof, or a pharmaceutically acceptable salt thereof, or a pharmaceutically acceptable solvate thereof; and from about 5 to about 50% by weight of a pharmaceutically acceptable excipient pharmaceutically acceptable excipient; and (ii) extragranular components comprising: from about 30 to about 90% by weight of a pharmaceutically acceptable excipient.

In another embodiment, provided herein is a pharmaceutical composition comprising (i) intragranular components comprising: from about 5 to about 10% by weight of the compound provided herein (e.g., compound A1 or A2), or an isotopic variant thereof, or a pharmaceutically acceptable salt thereof, or a pharmaceutically acceptable solvate thereof; and from about 10 to about 30% by weight of a pharmaceutically acceptable excipient pharmaceutically acceptable excipient; and (ii) extragranular components comprising: from about 60 to about 85% by weight of a pharmaceutically acceptable excipient.

In yet another embodiment, provided herein is a pharmaceutical composition comprising (i) intragranular components comprising: from about 6 to about 9% by weight of the compound provided herein (e.g., compound A1 or A2), or an isotopic variant thereof, or a pharmaceutically acceptable salt thereof, or a pharmaceutically acceptable solvate thereof; and from about 15 to about 25% by weight of a pharmaceutically acceptable excipient pharmaceutically acceptable excipient; and (ii) extragranular components comprising: from about 60 to about 80% by weight of a pharmaceutically acceptable excipient.

In certain embodiments, the intragranular excipient is a dispersant or carrier, a disintegrant, a filler, a glidant, a lubricant, an organic acid, a surfactant, or a mixture thereof. In certain embodiments, the extragranular excipient is a disintegrant, a filler, a glidant, a lubricant, an organic acid, a surfactant, or a mixture thereof.

In one embodiment, provided herein is a pharmaceutical composition comprising (i) intragranular components comprising: the compound provided herein (e.g., compound A1 or A2), or an isotopic variant thereof, or a pharmaceutically acceptable salt thereof, or a pharmaceutically acceptable solvate thereof; and a dispersant or carrier, a disintegrant, a filler, a glidant, a lubricant, or a mixture thereof; and (ii) extragranular components comprising: a disintegrant, a filler, a glidant, a lubricant, an organic acid, a surfactant, or a mixture thereof.

In another embodiment, provided herein is a pharmaceutical composition comprising (i) intragranular components comprising: the compound provided herein (e.g., compound A1 or A2), or an isotopic variant thereof, or a pharmaceutically acceptable salt thereof, or a pharmaceutically acceptable solvate thereof; and a dispersant or carrier, a glidant, and a lubricant; and (ii) extragranular components comprising: a disintegrant, a filler, a glidant, and a lubricant.

In yet another embodiment, provided herein is a pharmaceutical composition comprising (i) intragranular components comprising: the compound provided herein (e.g., compound A1 or A2), or an isotopic variant thereof, or a pharmaceutically acceptable salt thereof, or a pharmaceutically acceptable solvate thereof; and a dispersant or carrier, a glidant, and a lubricant; and (ii) extragranular components comprising: a disintegrant, a filler, a glidant, a lubricant, and an organic acid.

In yet another embodiment, provided herein is a pharmaceutical composition comprising (i) intragranular components comprising: the compound provided herein (e.g., compound A1 or A2), or an isotopic variant thereof, or a pharmaceutically acceptable salt thereof, or a pharmaceutically acceptable solvate thereof; and a dispersant or carrier, a glidant, and a lubricant; and (ii) extragranular components comprising: a disintegrant, a filler, a glidant, a lubricant, and a surfactant.

In yet another embodiment, provided herein is a pharmaceutical composition comprising (i) intragranular components comprising: the compound provided herein (e.g., compound A1 or A2), or an isotopic variant thereof, or a pharmaceutically acceptable salt thereof, or a pharmaceutically acceptable solvate thereof; and a dispersant or carrier, a glidant, and a lubricant; and (ii) extragranular components comprising: a disintegrant, a filler, a glidant, a lubricant, an organic acid and a surfactant.

In still another embodiment, provided herein is a pharmaceutical composition comprising (i) intragranular components comprising: the compound provided herein (e.g., compound A1 or A2), or an isotopic variant thereof, or a pharmaceutically acceptable salt thereof, or a pharmaceutically acceptable solvate thereof; and a dispersant or carrier, a disintegrant, a filler, a glidant, and a lubricant; and (ii) extragranular components comprising: a glidant and a lubricant.

In certain embodiments, the pharmaceutical composition provided herein comprises from about 5 to about 50%, from about 10 to about 50%, from about 15 to about 30%, or from about 15 to about 25% by weight of an intragranular dispersant or carrier. In certain embodiments, the pharmaceutical composition provided herein comprises about 15%, about 16%, about 17%, about 18%, about 19%, about 20%, about 21%, about 22%, about 23%, about 24%, about 25%, about 26%, about 27%, about 28%, about 29%, or about 30% by weight of an intragranular dispersant or carrier. In certain embodiments, the pharmaceutical composition provided herein comprises about 19% by weight of an intragranular dispersant or carrier. In certain embodiments, the pharmaceutical composition provided herein comprises about 25% by weight of an intragranular dispersant or carrier.

In certain embodiments, the intragranular dispersant or carrier is a hypromellose, a hypromellose acetate succinate, a hypromellose phthalate, a methacrylic acid and ethyl acrylate copolymer, a poloxamer, a polyethylene glycol, a povidone, a tocopherol polyethylene glycol succinate, or a mixture thereof. In certain embodiments, the intragranular dispersant or carrier is a povidone. In certain embodiments, the intragranular dispersant or carrier is a povidone having an average molecular weight of about 40,000 Da. In certain embodiments, the intragranular dispersant or carrier is PVP-K30. In certain embodiments, the intragranular dispersant or carrier is a hypromellose acetate succinate. In certain embodiments, the intragranular dispersant or carrier is a hypromellose acetate succinate having an average molecular weight of about 18,000 Da. In certain embodiments, the intragranular dispersant or carrier is HPMCAS, MF grade.

In certain embodiments, the pharmaceutical composition provided herein comprises from about 1 to about 50%, from about 2 to about 20%, or from about 5 to about 15% by weight of an intragranular disintegrant. In certain embodiments, the pharmaceutical composition provided herein comprises about 5%, about 6%, about 7%, about 8%, about 9%, about 10%, about 11%, about 12%, about 13%, about 14%, or about 15% by weight of an intragranular disintegrant. In certain embodiments, the pharmaceutical composition provided herein comprises about 6% by weight of an intragranular disintegrant. In certain embodiments, the pharmaceutical composition provided herein comprises about 10% by weight of an intragranular disintegrant. In certain embodiments, the intragranular disintegrant is a crosslinked polyvinyl pyrrolidone or croscarmellose sodium. In certain embodiments, the intragranular disintegrant is a crosslinked polyvinyl pyrrolidone. In certain embodiments, the intragranular disintegrant is POLYPLASDONE® XL. In certain embodiments, the intragranular disintegrant is croscarmellose sodium. In certain embodiments, the intragranular disintegrant is AC-DI-SOL®.

In certain embodiments, the pharmaceutical composition provided herein comprises from about 1 to about 50%, from about 2 to about 20%, from about 0.5 to about 15%, or from about 5 to about 15% by weight of an extragranular disintegrant. In certain embodiments, the pharmaceutical composition provided herein comprises about 5%, about 6%, about 7%, about 8%, about 9%, about 10%, about 11%, about 12%, about 13%, about 14%, or about 15% by weight of an extragranular disintegrant. In certain embodiments, the pharmaceutical composition provided herein comprises about 6% by weight of an extragranular disintegrant. In certain embodiments, the pharmaceutical composition provided herein comprises about 10% by weight of an extragranular disintegrant. In certain embodiments, the extragranular disintegrant is a crosslinked polyvinyl pyrrolidone or croscarmellose sodium. In certain embodiments, the extragranular disintegrant is a crosslinked polyvinyl pyrrolidone. In certain embodiments, the extragranular disintegrant is POLYPLASDONE® XL. In certain embodiments, the extragranular disintegrant is croscarmellose sodium. In certain embodiments, the extragranular disintegrant is AC-DI-SOL®.

In certain embodiments, the pharmaceutical composition provided herein comprises from about 10 to about 95%, from about 20 to about 90%, from about 25 to about 75%, or from about 30 to about 70% by weight of an intragranular filler. In certain embodiments, the pharmaceutical composition provided herein comprises about 25%, about 30%, about 35%, about 40%, about 45%, about 50%, about 55%, about 60%, about 65%, about 70%, or about 75% by weight of an intragranular filler. In certain embodiments, the pharmaceutical composition provided herein comprises about 30%, about 50%, or about 65% by weight of an intragranular filler. In certain embodiments, the intragranular filler is a microcrystalline cellulose, lactose, or a mixture thereof. In certain embodiments, the intragranular filler is a microcrystalline cellulose. In certain embodiments, the intragranular filler is AVICEL® PH 102. In certain embodiments, the intragranular filler is lactose. In certain embodiments, the intragranular filler is lactose FAST FLO® 316. In certain embodiments, the intragranular filler is a mixture of microcrystalline cellulose and lactose. In certain embodiments, the intragranular filler is a mixture of AVICEL® PH 102 and lactose FAST FLO® 316.

In certain embodiments, the pharmaceutical composition provided herein comprises from about 10 to about 95%, from about 20 to about 90%, from about 25 to about 75%, or from about 30 to about 70% by weight of an extragranular filler. In certain embodiments, the pharmaceutical composition provided herein comprises about 25%, about 30%, about 35%, about 40%, about 45%, about 50%, about 55%, about 60%, about 65%, about 70%, or about 75% by weight of an extragranular filler. In certain embodiments, the pharmaceutical composition provided herein comprises about 30%, about 50%, or about 65% by weight of an extragranular filler. In certain embodiments, the extragranular filler is a microcrystalline cellulose, lactose, or a mixture thereof. In certain embodiments, the extragranular filler is a microcrystalline cellulose. In certain embodiments, the extragranular filler is AVICEL® PH 102. In certain embodiments, the extragranular filler is lactose. In certain embodiments, the extragranular filler is lactose FAST FLO® 316. In certain embodiments, the extragranular filler is a mixture of microcrystalline cellulose and lactose. In certain embodiments, the extragranular filler is a mixture of AVICEL® PH 102 and lactose FAST FLO® 316.

In certain embodiments, the pharmaceutical composition provided herein comprises from about 0.1 to about 5%, from about 0.1 to about 2%, from about 0.2 to about 1.5%, or from about 0.2 to about 1% by weight of an intragranular glidant. In certain embodiments, the pharmaceutical composition provided herein comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% by weight of an intragranular glidant. In certain embodiments, the pharmaceutical composition provided herein comprises from about 0.1 to about 0.5% by weight of an intragranular glidant. In certain embodiments, the intragranular glidant is a colloidal silicon dioxide. In certain embodiment, the intragranular glidant is CAB-O-SIL®.

In certain embodiments, the pharmaceutical composition provided herein comprises from about 0.1 to about 5%, from about 0.1 to about 2%, from about 0.2 to about 1.5%, or from about 0.2 to about 1% by weight of an extragranular glidant. In certain embodiments, the pharmaceutical composition provided herein comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% by weight of an extragranular glidant. In certain embodiments, the pharmaceutical composition provided herein comprises from about 0.1 to about 0.5% by weight of an extragranular glidant. In certain embodiments, the extragranular glidant is a colloidal silicon dioxide. In certain embodiment, the extragranular glidant is CAB-O-SIL®.

In certain embodiments, the pharmaceutical composition provided herein comprises from about 0.01 to about 2%, from about 0.01 to about 1%, or from about 0.02 to about 0.5% by weight of an intragranular lubricant. In certain embodiments, the pharmaceutical composition provided herein comprises about 0.05%, about 0.08%, about 0.1%, about 0.2%, about 0.3%, about 0.4%, or about 0.5% by weight of an intragranular lubricant. In certain embodiments, the pharmaceutical composition provided herein comprises from about 0.05 to about 0.25% by weight of an intragranular lubricant. In certain embodiments, the intragranular lubricant is magnesium stearate.

In certain embodiments, the pharmaceutical composition provided herein comprises from about 0.01 to about 2%, from about 0.01 to about 1%, or from about 0.02 to about 0.5% by weight of am extragranular lubricant. In certain embodiments, the pharmaceutical composition provided herein comprises about 0.1%, about 0.2%, about 0.3%, about 0.4%, about 0.5%, about 0.6%, about 0.7%, about 0.8%, about 0.9%, or about 1% by weight of an extragranular lubricant. In certain embodiments, the pharmaceutical composition provided herein comprises from about 0.25 to about 0.5% by weight of an extragranular lubricant. In certain embodiments, the extragranular lubricant is magnesium stearate.

In certain embodiments, the pharmaceutical composition provided herein comprises from about 1 to about 25%, from about 2 to about 20%, from about 5 to about 15%, or from about 10 to about 15% by weight of an extragranular organic acid. In certain embodiments, the pharmaceutical composition provided herein comprises about 5%, about 6%, about 7%, about 8%, about 9%, about 10%, about 11%, about 12%, about 13%, about 14%, or about 15% by weight of an extragranular organic acid. In certain embodiments, the pharmaceutical composition provided herein comprises from about 10 to about 15% by weight of an extragranular organic acid. In certain embodiments, the extragranular organic acid is tartaric acid.

In certain embodiments, the pharmaceutical composition provided herein comprises from about 1 to about 30%, from about 2 to about 25%, or from about 5 to about 25% by weight of an extragranular surfactant. In certain embodiments, the pharmaceutical composition provided herein comprises about 10%, about 11%, about 12%, about 13%, about 14%, about 15%, about 16%, about 17%, about 18%, about 19%, or about 20% by weight of an extragranular surfactant. In certain embodiments, the pharmaceutical composition provided herein comprises from about 10 to about 20% by weight of an extragranular surfactant. In certain embodiments, the extragranular surfactant is sodium lauryl sulfate.

In one embodiment, provided herein is a pharmaceutical composition comprising (i) intragranular components comprising: from about 5 to about 20% by weight of the compound provided herein (e.g., compound A1 or A2), or an isotopic variant thereof, or a pharmaceutically acceptable salt thereof, or a pharmaceutically acceptable solvate thereof; from about 10 to about 25% by weight of a dispersant or carrier, from about 0.1 to about 5% by weight of a glidant, and from about 0.05 to about 2% by weight of a lubricant; and (ii) extragranular components comprising: from about 5 to about 25% by weight of a disintegrant, from about 25 to about 80% by weight of a filler, from about 0.1 to about 5% by weight of a glidant, and from about 0.05 to about 2% by weight of a lubricant.

In another embodiment, provided herein is a pharmaceutical composition comprising (i) intragranular components comprising: from about 5 to about 10% by weight of the compound provided herein (e.g., compound A1 or A2), or an isotopic variant thereof, or a pharmaceutically acceptable salt thereof, or a pharmaceutically acceptable solvate thereof; from about 15 to about 25% by weight of a dispersant or carrier, from about 0.2 to about 1.5% by weight of a glidant, and from about 0.05 to about 0.5% by weight of a lubricant; and (ii) extragranular components comprising: from about 5 to about 15% by weight of a disintegrant, from about 30 to about 70% by weight of a filler, from about 0.2 to about 2% by weight of a glidant, and from about 0.1 to about 1% by weight of a lubricant.

In yet another embodiment, provided herein is a pharmaceutical composition comprising (i) intragranular components comprising: from about 6 to about 9% by weight of the compound provided herein (e.g., compound A1 or A2), or an isotopic variant thereof, or a pharmaceutically acceptable salt thereof, or a pharmaceutically acceptable solvate thereof; from about 18 to about 25% by weight of a dispersant or carrier, from about 0.5 to about 1.5% by weight of a glidant, and from about 0.05 to about 0.3% by weight of a lubricant; and (ii) extragranular components comprising: from about 6 to about 10% by weight of a disintegrant, from about 30 to about 70% by weight of a filler, from about 0.2 to about 1% by weight of a glidant, and from about 0.1 to about 0.5% by weight of a lubricant.

In still another embodiment, provided herein is a pharmaceutical composition comprising (i) intragranular components comprising: from about 6 to about 9% by weight of the compound provided herein (e.g., compound A1 or A2), or an isotopic variant thereof, or a pharmaceutically acceptable salt thereof, or a pharmaceutically acceptable solvate thereof; from about 18 to about 25% by weight of a dispersant or carrier, from about 0.5 to about 1.5% by weight of a glidant, and from about 0.05 to about 0.3% by weight of a lubricant; and (ii) extragranular components comprising: about 10% by weight of a disintegrant, about 65% by weight of a filler, about 0.5% by weight of a glidant, and from about 0.2 to about 0.5% by weight of a lubricant.

In one embodiment, provided herein is a pharmaceutical composition comprising (i) intragranular components comprising: from about 5 to about 20% by weight of the compound of Formula A1; from about 10 to about 25% by weight of a polyvinyl pyrrolidone (e.g., PVP-K30), from about 0.1 to about 5% by weight of a colloidal silicon dioxide (e.g., CAB-O-SIL® M-5P), and from about 0.05 to about 2% by weight of magnesium stearate; and (ii) extragranular components comprising: from about 5 to about 25% by weight of a polyvinyl pyrrolidone (e.g., POLYPLASDONE® XL), from about 25 to about 80% by weight of a microcrystalline cellulose (e.g., AVICEL® PH 102), from about 0.1 to about 5% by weight of a glidant, and from about 0.05 to about 2% by weight of magnesium stearate.

In another embodiment, provided herein is a pharmaceutical composition comprising (i) intragranular components comprising: from about 5 to about 10% by weight of the compound of Formula A1; from about 15 to about 25% by weight of a polyvinyl pyrrolidone (e.g., PVP-K30), from about 0.2 to about 1.5% by weight of a colloidal silicon dioxide (e.g., CAB-O-SIL® M-5P), and from about 0.05 to about 0.5% by weight of magnesium stearate; and (ii) extragranular components comprising: from about 5 to about 15% by weight of a polyvinyl pyrrolidone (e.g., POLYPLASDONE® X1), from about 30 to about 70% by weight of a microcrystalline cellulose (e.g., AVICEL® PH 102), from about 0.2 to about 2% by weight of a glidant, and from about 0.1 to about 1% by weight of magnesium stearate.

In yet another embodiment, provided herein is a pharmaceutical composition comprising (i) intragranular components comprising: from about 6 to about 9% by weight of the compound of Formula A1; from about 18 to about 25% by weight of a polyvinyl pyrrolidone (e.g., PVP-K30), from about 0.5 to about 1.5% by weight of a colloidal silicon dioxide (e.g., CAB-O-SIL® M-5P), and from about 0.05 to about 0.3% by weight of magnesium stearate; and (ii) extragranular components comprising: from about 6 to about 10% by weight of a polyvinyl pyrrolidone (e.g., POLYPLASDONE® XL), from about 30 to about 70% by weight of a microcrystalline cellulose (e.g., AVICEL® PH 102), from about 0.2 to about 1% by weight of a colloidal silicon dioxide (e.g., CAB-O-SIL® M-5P), and from about 0.1 to about 0.5% by weight magnesium stearate.

In still another embodiment, provided herein is a pharmaceutical composition comprising (i) intragranular components comprising: from about 6 to about 9% by weight of the compound of Formula A1; from about 18 to about 25% by weight of a polyvinyl pyrrolidone (e.g., PVP-K30), from about 0.5 to about 1.5% by weight of a colloidal silicon dioxide (e.g., CAB-O-SIL® M-5P), and from about 0.05 to about 0.3% by weight of magnesium stearate; and (ii) extragranular components comprising: about 10% by weight of a polyvinyl pyrrolidone (e.g., POLYPLASDONE® XL), about 65% by weight of a microcrystalline cellulose (e.g., AVICEL® PH 102), about 0.5% by weight of a colloidal silicon dioxide (e.g., CAB-O-SIL® M-5P), and from about 0.2 to about 0.5% by weight of magnesium stearate.

In one embodiment, provided herein is a pharmaceutical composition comprising (i) intragranular components comprising: spray-dried particles provided herein and a pharmaceutically acceptable excipient; and (ii) extragranular components comprising: a pharmaceutically acceptable excipient.

In certain embodiments, the pharmaceutical composition provided herein comprises from about 10 to about 90%, from about 10 to about 50%, from about 20 to about 50%, or from about 25 to about 35% by weight of spray-dried particles as an intragranular component. In certain embodiments, the pharmaceutical composition provided herein comprises about 20%, about 21%, about 22%, about 23%, about 24%, about 25%, about 26%, about 27%, about 28%, about 29%, about 30%, about 31%, about 32%, about 33%, about 34%, or about 35% by weight of spray dried particles as an intragranular component. In certain embodiments, the pharmaceutical composition provided herein comprises from about 25 to about 35% by weight of spray dried particles as an intragranular component.

In certain embodiments, the pharmaceutical composition provided herein comprises from about 0.1 to about 5%, from about 0.1 to about 2%, from about 0.2 to about 1.5%, or from about 0.5 to about 1% by weight of an intragranular excipient. In certain embodiments, the pharmaceutical composition provided herein comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% by weight of an intragranular excipient.

In certain embodiments, the pharmaceutical composition provided herein comprises from about 10 to about 90%, from about 50 to about 90%, from about 50 to about 80%, or from about 60 to about 75% by weight of an intragranular excipient. In certain embodiments, the pharmaceutical composition provided herein comprises about 60%, about 62%, about 64%, about 65%, about 66%, about 68%, about 70%, about 72%, about 74%, about 75%, about 76%, about 78%, or about 80% by weight of an intragranular excipient. In certain embodiments, the pharmaceutical composition provided herein comprises about 65% by weight of an intragranular excipient. In certain embodiments, the pharmaceutical composition provided herein comprises about 75% by weight of an intragranular excipient.

In certain embodiments, the pharmaceutical composition provided herein comprises from about 0.1 to about 5%, from about 0.1 to about 2%, from about 0.2 to about 1.5%, or from about 0.5 to about 1% by weight of an extragranular excipient. In certain embodiments, the pharmaceutical composition provided herein comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% by weight of an extragranular excipient.

In certain embodiments, the pharmaceutical composition provided herein comprises from about 10 to about 90%, from about 50 to about 90%, from about 50 to about 80%, or from about 60 to about 75% by weight of an extragranular excipient. In certain embodiments, the pharmaceutical composition provided herein comprises about 60%, about 62%, about 64%, about 65%, about 66%, about 68%, about 70%, about 72%, about 74%, about 75%, about 76%, about 78%, or about 80% by weight of an extragranular excipient. In certain embodiments, the pharmaceutical composition provided herein comprises about 65% by weight of an extragranular excipient. In certain embodiments, the pharmaceutical composition provided herein comprises about 75% by weight of an extragranular excipient.

In one embodiment, provided herein is a pharmaceutical composition comprising (i) intragranular components comprising: from about 10 to about 50% by weight of spray-dried particles provided herein and from about 0.1 to about 10% by weight of a pharmaceutically acceptable excipient pharmaceutically acceptable excipient; and (ii) extragranular components comprising: from about 40 to about 90% by weight of a pharmaceutically acceptable excipient.

In another embodiment, provided herein is a pharmaceutical composition comprising (i) intragranular components comprising: from about 20 to about 50% by weight of spray-dried particles provided herein and from about 0.1 to about 5% by weight of a pharmaceutically acceptable excipient; and (ii) extragranular components comprising: from about 65 to about 80% by weight of a pharmaceutically acceptable excipient.

In yet another embodiment, provided herein is a pharmaceutical composition comprising (i) intragranular components comprising: from about 25 to about 35% by weight of spray-dried particles provided herein and from about 0.5 to about 2% by weight of a pharmaceutically acceptable excipient; and (ii) extragranular components comprising: from about 65 to about 75% by weight of a pharmaceutically acceptable excipient.

In certain embodiments, the intragranular excipient is a disintegrant, a filler, a glidant, a lubricant, an organic acid, a surfactant, or a mixture thereof. In certain embodiments, the extragranular excipient is a disintegrant, a filler, a glidant, a lubricant, an organic acid, a surfactant, or a mixture thereof.

In one embodiment, provided herein is a pharmaceutical composition comprising (i) intragranular components comprising: spray-dried particles provided herein, and a disintegrant, a filler, a glidant, a lubricant, or a mixture thereof; and (ii) extragranular components comprising: a disintegrant, a filler, a glidant, a lubricant, an organic acid, a surfactant, or a mixture thereof.

In another embodiment, provided herein is a pharmaceutical composition comprising (i) intragranular components comprising: spray-dried particles provided herein; and a glidant and a lubricant; and (ii) extragranular components comprising: a disintegrant, a filler, a glidant, and a lubricant.

In yet another embodiment, provided herein is a pharmaceutical composition comprising (i) intragranular components comprising: spray-dried particles provided herein; and a glidant and a lubricant; and (ii) extragranular components comprising: a disintegrant, a filler, a glidant, a lubricant, and an organic acid.

In yet another embodiment, provided herein is a pharmaceutical composition comprising (i) intragranular components comprising: spray-dried particles provided herein; and a glidant and a lubricant; and (ii) extragranular components comprising: a disintegrant, a filler, a glidant, a lubricant, and a surfactant.

In yet another embodiment, provided herein is a pharmaceutical composition comprising (i) intragranular components comprising: spray-dried particles provided herein; and a glidant and a lubricant; and (ii) extragranular components comprising: a disintegrant, a filler, a glidant, a lubricant, an organic acid and a surfactant.

In still another embodiment, provided herein is a pharmaceutical composition comprising (i) intragranular components comprising: spray-dried particles provided herein; and a disintegrant, a filler, a glidant, and a lubricant; and (ii) extragranular components comprising: a glidant and a lubricant.

In certain embodiments, the pharmaceutical composition provided herein comprises from about 1 to about 50%, from about 2 to about 20%, or from about 5 to about 15% by weight of an intragranular disintegrant. In certain embodiments, the pharmaceutical composition provided herein comprises about 5%, about 6%, about 7%, about 8%, about 9%, about 10%, about 11%, about 12%, about 13%, about 14%, or about 15% by weight of an intragranular disintegrant. In certain embodiments, the pharmaceutical composition provided herein comprises about 6% by weight of an intragranular disintegrant. In certain embodiments, the pharmaceutical composition provided herein comprises about 10% by weight of an intragranular disintegrant. In certain embodiments, the intragranular disintegrant is a crosslinked polyvinyl pyrrolidone or croscarmellose sodium. In certain embodiments, the intragranular disintegrant is a crosslinked polyvinyl pyrrolidone. In certain embodiments, the intragranular disintegrant is POLYPLASDONE® XL. In certain embodiments, the intragranular disintegrant is croscarmellose sodium. In certain embodiments, the intragranular disintegrant is AC-DI-SOL®.

In certain embodiments, the pharmaceutical composition provided herein comprises from about 1 to about 50%, from about 2 to about 20%, or from about 5 to about 15% by weight of an extragranular disintegrant. In certain embodiments, the pharmaceutical composition provided herein comprises about 5%, about 6%, about 7%, about 8%, about 9%, about 10%, about 11%, about 12%, about 13%, about 14%, or about 15% by weight of an extragranular disintegrant. In certain embodiments, the pharmaceutical composition provided herein comprises about 6% by weight of an extragranular disintegrant. In certain embodiments, the pharmaceutical composition provided herein comprises about 10% by weight of an extragranular disintegrant. In certain embodiments, the extragranular disintegrant is a crosslinked polyvinyl pyrrolidone or croscarmellose sodium. In certain embodiments, the extragranular disintegrant is a crosslinked polyvinyl pyrrolidone. In certain embodiments, the extragranular disintegrant is POLYPLASDONE® XL. In certain embodiments, the extragranular disintegrant is croscarmellose sodium. In certain embodiments, the extragranular disintegrant is AC-DI-SOL®.

In certain embodiments, the pharmaceutical composition provided herein comprises from about 10 to about 95%, from about 20 to about 90%, from about 25 to about 75%, or from about 30 to about 70% by weight of an intragranular filler. In certain embodiments, the pharmaceutical composition provided herein comprises about 25%, about 30%, about 35%, about 40%, about 45%, about 50%, about 55%, about 60%, about 65%, about 70%, or about 75% by weight of an intragranular filler. In certain embodiments, the pharmaceutical composition provided herein comprises about 30%, about 50%, or about 65% by weight of an intragranular filler. In certain embodiments, the intragranular filler is a microcrystalline cellulose, lactose, or a mixture thereof. In certain embodiments, the intragranular filler is a microcrystalline cellulose. In certain embodiments, the intragranular filler is AVICEL® PH 102. In certain embodiments, the intragranular filler is lactose. In certain embodiments, the intragranular filler is lactose FAST FLO® 316. In certain embodiments, the intragranular filler is a mixture of microcrystalline cellulose and lactose. In certain embodiments, the intragranular filler is a mixture of AVICEL® PH 102 and lactose FAST FLO® 316.

In certain embodiments, the pharmaceutical composition provided herein comprises from about 10 to about 95%, from about 20 to about 90%, from about 25 to about 75%, or from about 30 to about 70% by weight of an extragranular filler. In certain embodiments, the pharmaceutical composition provided herein comprises about 25%, about 30%, about 35%, about 40%, about 45%, about 50%, about 55%, about 60%, about 65%, about 70%, or about 75% by weight of an extragranular filler. In certain embodiments, the pharmaceutical composition provided herein comprises about 30%, about 50%, or about 65% by weight of an extragranular filler. In certain embodiments, the extragranular filler is a microcrystalline cellulose, lactose, or a mixture thereof. In certain embodiments, the extragranular filler is a microcrystalline cellulose. In certain embodiments, the extragranular filler is AVICEL® PH 102. In certain embodiments, the extragranular filler is lactose. In certain embodiments, the extragranular filler is lactose FAST FLO® 316. In certain embodiments, the extragranular filler is a mixture of microcrystalline cellulose and lactose. In certain embodiments, the extragranular filler is a mixture of AVICEL® PH 102 and lactose FAST FLO® 316.

In certain embodiments, the pharmaceutical composition provided herein comprises from about 0.1 to about 5%, from about 0.1 to about 2%, from about 0.2 to about 1.5%, or from about 0.2 to about 1% by weight of an intragranular glidant. In certain embodiments, the pharmaceutical composition provided herein comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% by weight of an intragranular glidant. In certain embodiments, the pharmaceutical composition provided herein comprises from about 0.1 to about 0.5% by weight of an intragranular glidant. In certain embodiments, the intragranular glidant is a colloidal silicon dioxide. In certain embodiment, the intragranular glidant is CAB-O-SIL®.

In certain embodiments, the pharmaceutical composition provided herein comprises from about 0.1 to about 5%, from about 0.1 to about 2%, from about 0.2 to about 1.5%, or from about 0.2 to about 1% by weight of an extragranular glidant. In certain embodiments, the pharmaceutical composition provided herein comprises about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5% by weight of an extragranular glidant. In certain embodiments, the pharmaceutical composition provided herein comprises from about 0.1 to about 0.5% by weight of an extragranular glidant. In certain embodiments, the extragranular glidant is a colloidal silicon dioxide. In certain embodiment, the extragranular glidant is CAB-O-SIL®.

In certain embodiments, the pharmaceutical composition provided herein comprises from about 0.01 to about 2%, from about 0.01 to about 1%, or from about 0.02 to about 0.5% by weight of an intragranular lubricant. In certain embodiments, the pharmaceutical composition provided herein comprises about 0.05%, about 0.08%, about 0.1%, about 0.2%, about 0.3%, about 0.4%, or about 0.5% by weight of an intragranular lubricant. In certain embodiments, the pharmaceutical composition provided herein comprises from about 0.05 to about 0.25% by weight of an intragranular lubricant. In certain embodiments, the intragranular lubricant is magnesium stearate.

In certain embodiments, the pharmaceutical composition provided herein comprises from about 0.01 to about 2%, from about 0.01 to about 1%, or from about 0.02 to about 0.5% by weight of an extragranular lubricant. In certain embodiments, the pharmaceutical composition provided herein comprises about 0.1%, about 0.2%, about 0.3%, about 0.4%, about 0.5%, about 0.6%, about 0.7%, about 0.8%, about 0.9%, or about 1% by weight of an extragranular lubricant. In certain embodiments, the pharmaceutical composition provided herein comprises from about 0.25 to about 0.5% by weight of an extragranular lubricant. In certain embodiments, the extragranular lubricant is magnesium stearate.

In certain embodiments, the pharmaceutical composition provided herein comprises from about 1 to about 25%, from about 2 to about 20%, from about 5 to about 15%, or from about 10 to about 15% by weight of an extragranular organic acid. In certain embodiments, the pharmaceutical composition provided herein comprises about 5%, about 6%, about 7%, about 8%, about 9%, about 10%, about 11%, about 12%, about 13%, about 14%, or about 15% by weight of an extragranular organic acid. In certain embodiments, the pharmaceutical composition provided herein comprises from about 10 to about 15% by weight of an extragranular organic acid. In certain embodiments, the extragranular organic acid is tartaric acid.

In certain embodiments, the pharmaceutical composition provided herein comprises from about 1 to about 30%, from about 2 to about 25%, or from about 5 to about 25% by weight of an extragranular surfactant. In certain embodiments, the pharmaceutical composition provided herein comprises about 10%, about 11%, about 12%, about 13%, about 14%, about 15%, about 16%, about 17%, about 18%, about 19%, or about 20% by weight of an extragranular surfactant. In certain embodiments, the pharmaceutical composition provided herein comprises from about 10 to about 20% by weight of an extragranular surfactant. In certain embodiments, the extragranular surfactant is sodium lauryl sulfate.

In one embodiment, provided herein is a pharmaceutical composition comprising (i) intragranular components comprising: from about 10 to about 50% by weight of spray-dried particles provided herein, from about 0.1 to about 5% by weight of a glidant, and from about 0.05 to about 2% by weight of a lubricant; and (ii) extragranular components comprising: from about 5 to about 25% by weight of a disintegrant, from about 25 to about 80% by weight of a filler, from about 0.1 to about 5% by weight of a glidant, and from about 0.05 to about 2% by weight of a lubricant.

In another embodiment, provided herein is a pharmaceutical composition comprising (i) intragranular components comprising: from about 20 to about 50% by weight of spray-dried particles provided herein, from about 0.2 to about 1.5% by weight of a glidant, and from about 0.05 to about 0.5% by weight of a lubricant; and (ii) extragranular components comprising: from about 5 to about 15% by weight of a disintegrant, from about 30 to about 70% by weight of a filler, from about 0.2 to about 2% by weight of a glidant, and from about 0.1 to about 1% by weight of a lubricant.

In yet another embodiment, provided herein is a pharmaceutical composition comprising (i) intragranular components comprising: from about 25 to about 35% by weight of spray-dried particles provided herein, from about 0.5 to about 1.5% by weight of a glidant, and from about 0.05 to about 0.3% by weight of a lubricant; and (ii) extragranular components comprising: from about 6 to about 10% by weight of a disintegrant, from about 30 to about 70% by weight of a filler, about 0.5% by weight of a glidant, and from about 0.1 to about 0.5% by weight of a lubricant.

In still another embodiment, provided herein is a pharmaceutical composition comprising (i) intragranular components comprising: from about 25 to about 35% by weight of spray-dried particles provided herein, from about 0.5 to about 1.5% by weight of a glidant, and from about 0.05 to about 0.3% by weight of a lubricant; and (ii) extragranular components comprising: about 10% by weight of a disintegrant, about 65% by weight of a filler, about 0.5% by weight of a glidant, and from about 0.2 to about 0.5% by weight of a lubricant.

In one embodiment, provided herein is a pharmaceutical composition comprising (i) intragranular components comprising: from about 10 to about 50% by weight of spray-dried particles provided herein, from about 0.1 to about 5% by weight of a colloidal silicon dioxide (e.g., CAB-O-SIL® M-5P), and from about 0.05 to about 2% by weight of magnesium stearate; and (ii) extragranular components comprising: from about 5 to about 25% by weight of a polyvinyl pyrrolidone (e.g., POLYPLASDONE® XL), from about 25 to about 80% by weight of a microcrystalline cellulose (e.g., AVICEL® PH 102), from about 0.1 to about 5% by weight of a colloidal silicon dioxide (e.g., CAB-O-SIL® M-5P), and from about 0.05 to about 2% by weight of magnesium stearate.

In another embodiment, provided herein is a pharmaceutical composition comprising (i) intragranular components comprising: from about 20 to about 50% by weight of spray-dried particles provided herein, from about 0.2 to about 1.5% by weight of a colloidal silicon dioxide (e.g., CAB-O-SIL® M-5P), and from about 0.05 to about 0.5% by weight of magnesium stearate; and (ii) extragranular components comprising: from about 5 to about 15% by weight of a polyvinyl pyrrolidone (e.g., POLYPLASDONE® XL), from about 30 to about 70% by weight of a microcrystalline cellulose (e.g., AVICEL® PH 102), from about 0.2 to about 2% by weight of a colloidal silicon dioxide (e.g., CAB-O-SIL® M-5P), and from about 0.1 to about 1% by weight of magnesium stearate.

In yet another embodiment, provided herein is a pharmaceutical composition comprising (i) intragranular components comprising: from about 25 to about 35% by weight of spray-dried particles provided herein, from about 0.5 to about 1.5% by weight of a colloidal silicon dioxide (e.g., CAB-O-SIL® M-5P), and from about 0.05 to about 0.3% by weight of magnesium stearate; and (ii) extragranular components comprising: from about 6 to about 10% by weight of a polyvinyl pyrrolidone (e.g., POLYPLASDONE® XL), from about 30 to about 70% by weight of a microcrystalline cellulose (e.g., AVICEL® PH 102), about 0.5% by weight of a colloidal silicon dioxide (e.g., CAB-O-SIL® M-5P), and from about 0.1 to about 0.5% by weight of magnesium stearate.

In still another embodiment, provided herein is a pharmaceutical composition comprising (i) intragranular components comprising: from about 25 to about 35% by weight of spray-dried particles provided herein, from about 0.5 to about 1.5% by weight a colloidal silicon dioxide (e.g., CAB-O-SIL® M-5P), and from about 0.05 to about 0.3% by weight of magnesium stearate; and (ii) extragranular components comprising: about 10% by weight of a polyvinyl pyrrolidone (e.g., POLYPLASDONE® XL), about 65% by weight of a microcrystalline cellulose (e.g., AVICEL® PH 102), about 0.5% by weight of a colloidal silicon dioxide (e.g., CAB-O-SIL® M-5P), and from about 0.2 to about 0.5% by weight of magnesium stearate.

In certain embodiments, the pharmaceutical composition provided herein comprises from about 1 to about 1,000 mg, from about 2 to about 500 mg, from about 5 to about 200 mg, from about 5 to about 100 mg, from about 5 to about 50 mg, from about 5 to about 25 mg, from about 5 to about 20 mg of the compound provided herein (e.g., compound A1 or A2), or an isotopic variant thereof, or a pharmaceutically acceptable salt thereof, or a pharmaceutically acceptable solvate thereof. In certain embodiments, the pharmaceutical composition provided herein comprises about 30 mg, about 35 mg, about 40 mg, about 45, about 50 mg, about 55 mg, about 60 mg, about 65 mg, about 70 mg, about 75 mg, about 80 mg, about 85 mg, about 90 mg, about 95 mg, or about 100 mg of the compound provided herein (e.g., compound A1 or A2), or an isotopic variant thereof, or a pharmaceutically acceptable salt thereof, or a pharmaceutically acceptable solvate thereof. In certain embodiments, the pharmaceutical composition provided herein comprises about 6 mg, about 7 mg, about 8 mg, about 9 mg, about 10 mg, about 11 mg, about 12 mg, about 13 mg, about 14, about 15 mg, about 16 mg, about 17 mg, about 18 mg, about 19 mg, about 20 mg, about 21 mg, about 22 mg, about 23 mg, about 24 mg, or about 25 mg of the compound provided herein (e.g., compound A1 or A2), or an isotopic variant thereof, or a pharmaceutically acceptable salt thereof, or a pharmaceutically acceptable solvate thereof.

In certain embodiments, the pharmaceutical composition provided herein further comprises a film-coating. In certain embodiments, the film-coating is ranging from about 0.1 to about 10%, from about 0.1 to about 5%, from about 0.2 to about 5%, or from about 0.5 to about 5% by the total weight of the composition

In certain embodiments, the pharmaceutical composition provided herein has a fast disintegration/dissolution. In certain embodiments, the pharmaceutical composition provided herein has a rate of disintegration/dissolution of no greater than about 60 min, no greater than about 30 min, no greater than about 10 min, no greater than about 5 min, no greater than about 4 min, no greater than about 3 min, no greater than about 2 min, no greater than about 1 min, or no greater than about 30 sec. In certain embodiments, the disintegration and dissolution of the pharmaceutical composition provided herein are measured according to Methods 701 and 711 in USP XXVI (2003), respectively.

In certain embodiments, the pharmaceutical composition provided herein has a fast disintegration. In certain embodiments, the pharmaceutical composition provided herein has a rate of disintegration (for example, a complete disintegration, a substantially complete disintegration, about 90% disintegration, about 80% disintegration, about 70% disintegration, about 60% disintegration, or about 50% disintegration) of no greater than about 60 min, no greater than about 30 min, no greater than about 10 min, no greater than about 5 min, no greater than about 4 min, no greater than about 3 min, no greater than about 2 min, no greater than about 1 min, or no greater than about 30 sec.

In certain embodiments, the pharmaceutical composition provided herein has a fast dissolution. In certain embodiments, the pharmaceutical composition provided herein has a rate of dissolution (for example, releasing no less than about 50%, about 60%, about 70%, about 80%, about 90%, or about 95% of the active ingredient) of no greater than about 60 min, no greater than about 30 min, no greater than about 10 min, no greater than about 5 min, no greater than about 4 min, no greater than about 3 min, no greater than about 2 min, no greater than about 1 min, or no greater than about 30 sec.

In certain embodiments, the pharmaceutical composition provided herein has no food effect. In certain embodiments, the pharmaceutical composition provided herein has a bioavailability under fasted conditions that differs no greater than 10%, no greater than 9%, no greater than 8%, no greater than 7%, no greater than 6%, no greater than 5%, no greater than 4%, no greater than 3%, no greater than 2%, or no greater than 1% from the bioavailability under fed conditions. In certain embodiments, the pharmaceutical composition provided herein has a bioavailability under fasted conditions that differs no greater than 5%, no greater than 4%, no greater than 3%, no greater than 2%, or no greater than 1% from the bioavailability under fed conditions.

In certain embodiments, the pharmaceutical composition provided herein has a bioavailability of no less than about 5% F, no less than about 10% F, no less than about 20% F, no less than about 25% F, no less than about 30% F, no less than about 35% F, or no less than about 40% F. In certain embodiments, the pharmaceutical composition provided herein has a bioavailability of no less than about 30% F, no less than about 35% F, or no less than about 40% F. In certain embodiments, the pharmaceutical composition provided herein has a bioavailability of about 30% F, about 31% F, about 32% F, about 33% F, about 34% F, about 35% F, about 36% F, about 37% F, about 38% F, about 39% F, or about 40% F.

In certain embodiments, the pharmaceutical composition provided herein is stable at a 25° C. and 60% relative humidity for a period ranging from about 6 months to about 10 years, from about 6 months to about 5 years, from about 6 months to about 2 years, from about 12 months to about 24 months. In certain embodiments, the pharmaceutical composition provided herein is stable at a 25° C. and 60% relative humidity for a period from about 6 months to about 12 months, from about 6 months to about 18 months, or from about 6 months to about 24 months. In certain embodiments, the pharmaceutical composition provided herein is at a 25° C. and 60% relative humidity for at least 6 months, at least 12 months, at least 18 months, at least 24 months, or at least 5 years.

The pharmaceutical compositions provided herein can be provided in a unit-dosage form or multiple-dosage form. A unit-dosage form, as used herein, refers to physically discrete a unit suitable for administration to a human and animal subject, and packaged individually as is known in the art. Each unit-dose contains a predetermined quantity of an active ingredient(s) sufficient to produce the desired therapeutic effect, in association with the required pharmaceutical carriers or excipients. Examples of a unit-dosage form include an ampoule, syringe, and individually packaged tablet and capsule. For example, a 100 mg unit dose contains about 100 mg of an active ingredient in a packaged tablet or capsule. A unit-dosage form may be administered in fractions or multiples thereof. A multiple-dosage form is a plurality of identical unit-dosage forms packaged in a single container to be administered in segregated unit-dosage form. Examples of a multiple-dosage form include a vial, bottle of tablets or capsules, or bottle of pints or gallons.

The pharmaceutical compositions provided herein can be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In certain embodiments, the pharmaceutically acceptable excipient used in the pharmaceutical compositions (e.g., granular formulations, tablet formulations, or capsule formulations) provided herein is a binder, granulator, filler, diluent, disintegrant, lubricant, glidant, coloring agent, flavoring agent, sweetening, preservative, organic acid, surfactant, or a mixture thereof.

In certain embodiments, suitable binders or granulators for use in the pharmaceutical compositions provided herein include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof.

Suitable fillers for use in the pharmaceutical compositions provided herein include, but are not limited to, talc, calcium carbonate, microcrystalline cellulose, powdered cellulose, dextrates, kaolin, mannitol, silicic acid, sorbitol, starch, pre-gelatinized starch, and mixtures thereof.

In certain embodiments, suitable diluents for use in the pharmaceutical compositions provided herein include, but are not limited to, dicalcium phosphate, calcium sulfate, lactose, sorbitol, sucrose, inositol, cellulose, kaolin, mannitol, sodium chloride, dry starch, and powdered sugar.

In certain embodiments, suitable disintegrants for use in the pharmaceutical compositions provided herein include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof.

In certain embodiments, suitable lubricants for use in the pharmaceutical compositions provided herein include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof.

In certain embodiments, suitable glidants for use in the pharmaceutical compositions provided herein include, but are not limited to, colloidal silicon dioxide, CAB-O-SIL® (Cabot Co. of Boston, Mass.), and asbestos-free talc.

In certain embodiments, suitable coloring agents for use in the pharmaceutical compositions provided herein include, but are not limited to, any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye.

In certain embodiments, suitable flavoring agents for use in the pharmaceutical compositions provided herein include, but are not limited to, natural flavors extracted from plants, such as fruits, and synthetic blends of compounds which produce a pleasant taste sensation, such as peppermint and methyl salicylate. Suitable sweetening agents for use in the pharmaceutical compositions provided herein include, but are not limited to, sucrose, lactose, mannitol, syrups, glycerin, and artificial sweeteners, such as saccharin and aspartame. In certain embodiments, suitable preservatives for use in the pharmaceutical compositions provided herein include, but are not limited to, glycerin, methyl and propylparaben, benzoic add, sodium benzoate and alcohol. In certain embodiments, suitable organic acids for use in the pharmaceutical compositions provided herein include, but are not limited to, citric and tartaric acid.

The pharmaceutical compositions provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The pharmaceutical compositions provided herein for oral administration can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The solid dosage forms provided herein may be encapsulated in a capsule. The capsules may also be coated as known by those of skill in the art in order to modify or sustain dissolution of the active ingredient.

The pharmaceutical compositions provided herein for oral administration can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

The pharmaceutical compositions provided herein for oral administration can be formulated as immediate or modified release dosage forms, including delayed-, sustained, pulsed-, controlled, targeted-, and programmed-release forms.

The pharmaceutical compositions provided herein in a modified release dosage form can be fabricated as a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates can be made by the processes known to those skilled in the art, including wet- and dry-granulation, extrusion/spheronization, roller-compaction, melt-congealing, and by spray-coating seed cores. See, for example, Multiparticulate Oral Drug Delivery; Ghebre-Sellassie Ed.; Marcel Dekker: 1994; and Pharmaceutical Pelletization Technology; Ghebre-Sellassie Ed.; Marcel Dekker: 1989.

Other excipients or carriers as described herein can be blended with the pharmaceutical compositions to aid in processing and forming the multiparticulates. The resulting particles can themselves constitute the multiparticulate device or can be coated by various film-forming materials, such as enteric polymers, water-swellable, and water-soluble polymers. The multiparticulates can be further processed as a capsule or a tablet.

### Methods of Use

In one embodiment, provided herein is a method for treating or preventing a Flaviviridae infection in a subject, which comprises administering to the subject a therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein.

In certain embodiments, the Flaviviridae infection is a pestivirus infection. In certain embodiments, the Flaviviridae infection is a flavivirus infection. In certain embodiments, the Flaviviridae infection is a hepacivirus infection. In certain embodiments, the Flaviviridae infection is an HCV infection.

In another embodiment, provided herein is a method for treating, preventing, or ameliorating one or more symptoms of a disease or disorder associated with a Flaviviridae infection in a subject, comprising administering to the subject a therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein. In certain embodiments, the disease or disorder associated with a Flaviviridae infection is West Nile virus, dengue fever, yellow fever, chronic hepatitis, cirrhosis, hepatocarcinoma, or extra hepatic manifestation.

In yet another embodiment, provided herein is a method for treating, preventing, or ameliorating one or more symptoms of a liver disease or disorder associated with an HCV infection in a subject, comprising administering to the subject a therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein. In certain embodiments, the liver disease or disorder associated with an HCV infection is chronic hepatitis, cirrhosis, hepatocarcinoma, or extra hepatic manifestation.

In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is at least about 1 mg per day, at least about 5 mg per day, at least about 10 mg per day, or at least about 20 mg per day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is at least about 1 mg per day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is at least about 5 mg per day. In certain embodiments, the therapeutically effective amount the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is at least about 10 mg per day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is at least about 20 mg per day.

In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is ranging from about 1 to about 1,000 mg per day, from about 1 to about 500 mg per day, from about 5 to about 500 mg per day, from about 5 to about 200 mg per day, from about 5 to about 100 mg per day, or from about 10 to about 100 mg per day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is ranging from about 1 to about 1,000 mg per day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is ranging from about 1 to about 500 mg per day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is ranging from about 5 to about 500 mg per day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is ranging from about 5 to about 200 mg per day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is ranging from about 5 to about 100 mg per day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is ranging from about 10 to about 100 mg per day.

In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is about 1 mg per day, about 2 mg per day, about 5 mg per day, about 10 mg per day, about 25 mg per day, about 50 mg per day, about 100 mg per day, about 200 mg per day, about 500 mg per day, or about 1,000 mg per day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is about 1 mg per day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is about 2 mg per day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is about 5 mg per day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is about 10 mg per day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is about 25 mg per day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is about 50 mg per day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is about 100 mg per day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is about 200 mg per day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is about 500 mg per day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is about 1,000 mg per day.

In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is about 1 mg per day, about 5 mg per day, about 10 mg per day, about 25 mg per day, about 50 mg per day, or about 100 mg per day.

In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is ranging from about 0.02 to about 20 mg/kg/day, from about 0.1 to about 10 mg/kg/day, from about 0.1 to about 5 mg/kg/day, or from about 0.2 to about 2 mg/kg/day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is ranging from about 0.02 to about 20 mg/kg/day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is ranging from about 0.1 to about 10 mg/kg/day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is ranging from about 0.1 to about 5 mg/kg/day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is ranging from about 0.2 to about 2 mg/kg/day.

In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is about 0.02 mg/kg/day, about 0.1 mg/kg/day, about 0.2 mg/kg/day, about 0.5 mg/kg/day, about 1 mg/kg/day, or about 2 mg/kg/day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is about 0.02 mg/kg/day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is about 0.1 mg/kg/day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is about 0.2 mg/kg/day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is about 0.5 mg/kg/day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is about 1 mg/kg/day. In certain embodiments, the therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is about 2 mg/kg/day.

In certain embodiments, the hepatitis C virus (HCV) is drug-resistant.

Thus, in one embodiment, provided herein is a method for treating or preventing a drug-resistant hepatitis C viral infection in a subject, which comprises administering to the subject a therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, wherein the therapeutically effective amount is as defined herein.

In another embodiment, provided herein is a method for treating, preventing, or ameliorating one or more symptoms of a liver disease or disorder associated with a drug-resistant HCV infection in a subject, comprising administering to the subject a therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, wherein the therapeutically effective amount is as defined herein. In certain embodiments, the liver disease or disorder associated with a drug-resistant HCV infection is chronic hepatitis, cirrhosis, hepatocarcinoma, or extra hepatic manifestation.

In certain embodiments, the drug-resistant HCV is resistant to an anti-HCV agent. In certain embodiments, the anti-HCV agent is an interferon. In certain embodiments, the anti-HCV agent is ribavirin. In certain embodiments, the anti-HCV agent is amantadine. In certain embodiments, the anti-HCV agent is an interleukin. In certain embodiments, the anti-HCV agent is a phenanthrenequinone. In certain embodiments, the anti-HCV agent is a thiazolidine. In certain embodiments, the anti-HCV agent is a benzanilide. In certain embodiments, the anti-HCV agent is a helicase inhibitor. In certain embodiments, the anti-HCV agent is a nucleotide analogue. In certain embodiments, the anti-HCV agent is a gliotoxin. In certain embodiments, the anti-HCV agent is a cerulenin. In certain embodiments, the anti-HCV agent is an antisense phopshorothioate ologodexoynucleotide. In certain embodiments, the anti-HCV agent is an inhibitor of IRES-dependent translation. In certain embodiments, the anti-HCV agent is a ribozyme. In certain embodiments, the anti-HCV agent is a cyclophilin inhibitor. In certain embodiments, the anti-HCV agent is SYC-635.

In certain embodiments, the anti-HCV agent is a protease inhibitor. In certain embodiments, the anti-HCV agent is a cysteine protease inhibitor. In certain embodiments, the anti-HCV agent is a caspase inhibitor. In certain embodiments, the anti-HCV agent is GS 9450. In certain embodiments, the anti-HCV agent is a serine protease inhibitor. In certain embodiments, the anti-HCV agent is an NS3/4A serine protease inhibitor. In certain embodiments, the anti-HCV agent is a serine protease inhibitor selected from ABT-450, faldaprevir (BI-201335), asunaprevir (BMS-650032), boceprevir (SCH 503034), danoprevir (ITMN-191/R7227), GS-9256, IDX136, IDX316, IDX320, MK-5172, SCH900518, telaprevir (VX-950), TMC 435, vaniprevir (MK-7009), VX-985, and mixtures thereof.

In certain embodiments, the anti-HCV agent is a polymerase inhibitor. In certain embodiments, the anti-HCV agent is an NS5B polymerase inhibitor. In certain embodiments, the anti-HCV agent is a polymerase inhibitor selected from ABT-072, ABT-333, AG-02154, ANA598, ANA773, deleobuvir (BI 207127), GS-9190, HCV-796, IDX184, IDX375, JTK-109, MK-0608, MK-3281, NM283, PF-868554, PSI-879, PSI-938, PSI-6130, PSI-7851, sofosbuvir (PSI-7977), R1626, R7128, RG7128, VCH-759, VCH-916, VX-222 (VCH-222), and mixtures thereof. In certain embodiments, the NS5B polymerase inhibitor is a nucleotide inhibitor. In certain embodiments, the NS5B polymerase inhibitor is a 2′-C-methylnucleoside. In certain embodiments, the NS5B polymerase inhibitor is a 2′-C-methyl ribonucleoside. In certain embodiments, the NS5B polymerase inhibitor is a 2′-F-2′-C-methylnucleoside. In certain embodiments, the NS5B polymerase inhibitor is a 2′-F-2′-C-methyl ribonucleoside. In certain embodiments, the NS5B polymerase inhibitor is a non-nucleoside inhibitor. In certain embodiments, the NS5B polymerase inhibitor is a benzofuran, benzothiadiazine, or thiophene.

In certain embodiments, the anti-HCV agent is an NS5A inhibitor. In certain embodiments, the anti-HCV agent is an NS5A inhibitor selected from daclatasvir (BMS-790052), BMS-824393, ledipasvir (GS-5885), GS-5816, GSK2336805, PPI-668, and mixtures thereof.

In certain embodiments, the drug-resistance of the HCV infection is caused by an HCV variant. In certain embodiments, the HCV variant contains an NS3 protein variant. In certain embodiments, the NS3 protein variant contains a mutation or deletion. In certain embodiments, the NS3 protein variant contains one or more mutations and/or deletions at the amino acid positions of 9, 16, 18, 23, 36, 39, 40, 41, 43, 54, 55, 65, 67, 70, 71, 80, 89, 109, 138, 155, 156, 162, 168, 170, 174, 176, 179, 260, and 489. In certain embodiments, the NS3 protein variant contains one or more mutations and/or deletions at the amino acid positions of 16, 23, 36, 39, 41, 43, 54, 55, 80, 89, 109, 138, 155, 156, 168, 170, 174, 176, 260, and 489. In certain embodiments, the NS3 protein variant contains one or more mutations and/or deletions at the amino acid positions of 36, 54, 155, 156, 168, and 170. In certain embodiments, the NS3 protein variant contains one, two, or more mutations and/or deletions, each independently selected from C16S, V23A, V36A, V36G, V36L, V36M, A39V, Q41R, F43C, F43I, F43S, F43V, T54A, T54S, V55A, Q80K, Q80G, Q80H, Q80L, Q80R, P89R, R109K, S138T, R155G, R155I, R155K, R155L, R155M, R155Q, R155S, R155T, A156G, A156I, A156S, A156T, A156V, D168A, D168E, D168G, D168H, D168I, D168N, D168T, D168V, D168Y, V170A, V170T, S174K, S174N, E176K, T260A, and S489L, provided that there is only one mutation or deletion at a given amino acid position in the NS3 protein variant. In certain embodiments, the NS3 protein variant contains one, two, or more mutations and/or deletions, each independently selected from R155K, A156S, A156T, D168V, and T260A, provided that there is only one mutation or deletion at a given amino acid position in the NS3 protein variant.

In certain embodiments, the HCV variant contains an NS4A protein variant. In certain embodiments, the NS4A protein variant contains a mutation or deletion. In certain embodiments, the NS4A protein variant contains a mutation at the amino acid position of 23. In certain embodiments, the NS4A protein variant contains the V23A mutation.

In certain embodiments, the HCV variant contains an NS4B protein variant. In certain embodiments, the NS4B protein variant contains a mutation or deletion. In certain embodiments, the NS4B protein variant contains a mutation at the amino acid position of 15. In certain embodiments, the NS4B protein variant contains the E15G mutation.

In certain embodiments, the HCV variant contains an NS5A protein variant. In certain embodiments, the NS5A protein variant contains a mutation or deletion. In certain embodiments, the NS5A protein variant contains one or more mutations and/or deletions at the amino acid positions of 23, 28, 30, 31, 32, 37, 54, 58, 63, and 93. In certain embodiments, the NS5A protein variant contains one or more mutations and/or deletions at the amino acid positions of 23, 24, 28, 30, 31, 32, 37, 54, 58, 63, 93, 295, 318, 320, 356, 404, and 442. In certain embodiments, the NS5A protein variant contains one or more mutations and/or deletions at the amino acid positions of 24, 28, 30, 31, 32, 54, 93, 295, and 318. In certain embodiments, the NS5A protein variant contains one, two, or more mutations and/or deletions, each independently selected from L23F, L28M, L28T, M28T, AQ30, Q30E, Q30H, Q30K, Q30R, AR30, R30E, R30Q, L31F, L31M, L31V, P32L, F37L, H54Y, Q54H, P58H, P58S, I63V, Y93C, Y93H, Y93N, and Y93S, provided that there is only one mutation or deletion at a given amino acid position in the NS5A protein variant. In certain embodiments, the NS5A protein variant contains one, two, or more mutations and/or deletions, each independently selected from L23F, K24E, L28M, L28T, M28T, AQ30, Q30E, Q30H, Q30K, Q30R, AR30, R30E, R30Q, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from L23F, K24E, L28M, L28T, AQ30, Q30E, Q30H, Q30K, Q30R, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from L23F, K24E, M28T, AR30, R30E, R30Q, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from K24E, M28T, Q30E, Q30H, Q30K, Q30R, L31F, L31M, L31V, P32L, Y93C, Y93H, Y93N, E295G, and R318W, provided that there is only one mutation or deletion at a given amino acid position in the NS5A protein variant.

In certain embodiments, the subject being treated with a method provided herein is infected with a drug resistant genotype 2 HCV. In certain embodiments, the drug resistant genotype 2 HCV contains a NS5A protein mutation. In certain embodiments, the drug resistant genotype 2 HCV contains the L31M mutation in the NS5A protein.

In certain embodiments, the HCV variant contains an NS5B protein variant. In certain embodiments, the NS5B protein variant contains a mutation or deletion. In certain embodiments, the NS5B protein variant contains one or more mutations and/or deletions at the amino acid positions of 15, 95, 96, 142, 152, 156, 222, 223, 244, 282, 309, 310, 316, 320, 321, 326, 329, 333, 365, 411, 414, 415, 423, 445, 448, 451, 452, 495, 554, 558, and 559. In certain embodiments, the NS5B protein variant contains one or more mutations and/or deletions at the amino acid positions of 316, 414, and 423. In certain embodiments, the NS5B protein variant contains one, two, or more mutations and/or deletions, each independently selected from S15G, H95Q, H95R, S96T, N142T, G152E, P156L, R222Q, C223H, C223Y, D244N, S282T, Q309R, D310N, C316N, C316S, C316Y, L320I, V321I, S326G, T329I, A333E, S365A, S365T, N411S, M414I, M414L, M414T, F415Y, M423I, M423T, M423V, C445F, Y448H, C451R, Y452H, P495A, P495I, G554D, G554S, G558R, D559G, D559N, and D559S, provided that there is only one mutation or deletion at a given amino acid position in the NS5B protein variant. In certain embodiments, the NS5B protein variant contains one, two, or more mutations and/or deletions, each independently selected from C316Y, M414T, and M423T, provided that there is only one mutation or deletion at a given amino acid position in the NS5B protein variant.

In one embodiment, provided herein is a method for treating or preventing a hepatitis C virus infection in a subject, comprising administering to the subject the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein; in an amount that is sufficient to provide a plasma concentration of the compound at steady state in the range from about 1 nM to about 1 μM, from about 2 nM to about 500 nM, from about 2 nM to about 200 nM, from about 2 nM to about 100 nM, or from about 2 nM to about 50 nM. In one embodiment, the amount of the compound (e.g., compound A1 or A2) administered is sufficient to provide a plasma concentration of the compound at steady state in the range from about 1 nM to about 1 μM. In another embodiment, the amount of the compound (e.g., compound A1 or A2) administered is sufficient to provide a plasma concentration of the compound at steady state in the range from about 2 nM to about 500 nM. In yet another embodiment, the amount of the compound (e.g., compound A1 or A2) administered is sufficient to provide a plasma concentration of the compound at steady state in the range from about 2 nM to about 200 nM. In yet another embodiment, the amount of the compound (e.g., compound A1 or A2) administered is sufficient to provide a plasma concentration of the compound at steady state in the range from about 2 nM to about 100 nM. In still another embodiment, the amount of the compound (e.g., compound A1 or A2) administered is sufficient to provide a plasma concentration of the compound at steady state in the range from about 2 nM to about 50 nM. As used herein, the term “plasma concentration at steady state” is the concentration reached after a period of administration of a compound. Once steady state is reached, there are minor peaks and troughs on the time dependent curve of the plasma concentration of the compound (e.g., compound A1 or A2).

In another embodiment, provided herein is a method for treating or preventing a hepatitis C virus infection in a subject, comprising administering to the subject the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein; in an amount that is sufficient to provide a peak plasma concentration (a maximum plasma concentration) of the compound ranging from about 5 nM to about 1 μM, from about 5 nM to about 500 nM, from about 10 nM to about 200 nM, about 10 nM to about 100 nM, or from about 50 nM to about 100 nM. In one embodiment, the amount of the compound (e.g., compound A1 or A2) administered is sufficient to provide a peak plasma concentration of the compound ranging from about 5 nM to about 1 μM. In another embodiment, the amount of the compound (e.g., compound A1 or A2) administered is sufficient to provide a peak plasma concentration of the compound ranging from about 5 nM to about 500 nM. In yet another embodiment, the amount of the compound (e.g., compound A1 or A2) administered is sufficient to provide a peak plasma concentration of the compound ranging from about 10 nM to about 200 nM. In yet another embodiment, the amount of the compound (e.g., compound A1 or A2) administered is sufficient to provide a peak plasma concentration of the compound ranging from about 10 nM to about 100 nM. In still another embodiment, the amount of the compound (e.g., compound A1 or A2) administered is sufficient to provide a peak plasma concentration of the compound ranging from about 50 nM to about 100 nM.

In yet another embodiment, provided herein is a method for treating or preventing a hepatitis C virus infection in a subject, comprising administering to the subject the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein; in an amount that is sufficient to provide a trough plasma concentration (a minimum plasma concentration) of the compound ranging from about 1 nM to about 500 nM, from about 2 nM to about 200 nM, from about 5 nM to about 100 nM, from about 1 nM to about 50 nM, from about 10 nM to about 50 nM, from about 1 nM to about 20 nM, or from about 1 nM to about 10 nM. In one embodiment, the amount of the compound (e.g., compound A1 or A2) administered is sufficient to provide a trough plasma concentration of the compound ranging from about 1 nM to about 500 nM. In another embodiment, the amount of the compound (e.g., compound A1 or A2) administered is sufficient to provide a trough plasma concentration of the compound ranging from about 2 nM to about 200 nM. In yet another embodiment, the amount of the compound (e.g., compound A1 or A2) administered is sufficient to provide a trough plasma concentration of the compound ranging from about 5 nM to about 100 nM. In yet another embodiment, the amount of the compound (e.g., compound A1 or A2) administered is sufficient to provide a trough plasma concentration of the compound ranging from about 1 nM to about 50 nM. In yet another embodiment, the amount of the compound (e.g., compound A1 or A2) administered is sufficient to provide a trough plasma concentration of the compound ranging from about 10 nM to about 50 nM. In yet another embodiment, the amount of the compound (e.g., compound A1 or A2) administered is sufficient to provide a trough plasma concentration of the compound ranging from about 1 nM to about 20 nM. In still another embodiment, the amount of the compound (e.g., compound A1 or A2) administered is sufficient to provide a trough plasma concentration of the compound ranging from about 1 nM to about 10 nM.

In yet another embodiment, provided herein is a method for treating or preventing a hepatitis C virus infection in a subject, comprising administering to the subject the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein; in an amount that is sufficient to provide an area under the curve (AUC) of the compound in the range from about 100 to about 10,000 ng·hr/mL, from about 100 to 5,000 ng·hr/mL, from about 100 to 2,000 ng·hr/mL, from about 200 to 2,000 ng·hr/mL, or from about 500 to 2,000 ng·hr/mL. In one embodiment, the amount of the compound (e.g., compound A1 or A2) administered is sufficient to provide an AUC of the compound in the range from about 100 to about 10,000 ng·hr/mL. In another embodiment, the amount of the compound (e.g., compound A1 or A2) administered is sufficient to provide an AUC of the compound in the range from about 100 to 5,000 ng·hr/mL. In yet another embodiment, the amount of the compound (e.g., compound A1 or A2) administered is sufficient to provide an AUC of the compound in the range from about 100 to 2,000 ng·hr/mL. In yet another embodiment, the amount of the compound (e.g., compound A1 or A2) administered is sufficient to provide an AUC of the compound in the range from about 200 to 2,000 ng·hr/mL. In still another embodiment, the amount of the compound (e.g., compound A1 or A2) administered is sufficient to provide an AUC of the compound in the range from about 200 to 2,000 ng·hr/mL.

In yet another embodiment, provided herein is a method for treating, preventing, or ameliorating one or more symptoms of a liver disease or disorder associated with an HCV infection in a subject, comprising administering to the subject the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein; in an amount that is sufficient to provide a plasma concentration of the compound at steady state in the range from about 1 nM to about 1 μM, from about 2 nM to about 500 nM, from about 5 nM to about 200 nM, from about 10 nM to about 100 nM, or from about 10 nM to about 50 nM. In one embodiment, the amount of the compound (e.g., compound A1 or A2) administered is sufficient to provide a plasma concentration of the compound at steady state in the range from about 1 nM to about 1 μM. In another embodiment, the amount of the compound (e.g., compound A1 or A2) administered is sufficient to provide a plasma concentration of the compound at steady state in the range from about 2 nM to about 500 nM. In yet another embodiment, the amount of the compound (e.g., compound A1 or A2) administered is sufficient to provide a plasma concentration of the compound at steady state in the range from about 5 nM to about 200 nM. In yet another embodiment, the amount of the compound (e.g., compound A1 or A2) administered is sufficient to provide a plasma concentration of the compound at steady state in the range from about 10 nM to about 100 nM. In still another embodiment, the amount of the compound (e.g., compound A1 or A2) administered is sufficient to provide a plasma concentration of the compound at steady state in the range from about 10 nM to about 50 nM.

In yet another embodiment, provided herein is a method for treating, preventing, or ameliorating one or more symptoms of a liver disease or disorder associated with an HCV infection in a subject, comprising administering to the subject the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein; in an amount that is sufficient to provide a peak plasma concentration of the compound ranging from about 5 nM to about 1 μM, from about 10 nM to about 500 nM, from about 20 nM to about 200 nM, or from about 50 nM to about 100 nM. In one embodiment, the amount of the compound (e.g., compound A1 or A2) administered is sufficient to provide a peak plasma concentration of the compound ranging from about 5 nM to about 1 μM. In another embodiment, the amount of the compound (e.g., compound A1 or A2) administered is sufficient to provide a peak plasma concentration of the compound ranging from about 10 nM to about 500 nM. In yet another embodiment, the amount of the compound (e.g., compound A1 or A2) administered is sufficient to provide a peak plasma concentration of the compound ranging from about 20 nM to about 200 nM. In still another embodiment, the amount of the compound (e.g., compound A1 or A2) administered is sufficient to provide a peak plasma concentration of the compound ranging from about 50 nM to about 100 nM.

In yet another embodiment, provided herein is a method for treating, preventing, or ameliorating one or more symptoms of a liver disease or disorder associated with an HCV infection in a subject, comprising administering to the subject the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein; in an amount that is sufficient to provide a trough plasma concentration of the compound ranging from about 1 nM to about 500 nM, from about 2 nM to about 200 nM, from about 5 nM to about 100 nM, from about 10 nM to about 50 nM. In one embodiment, the amount of the compound (e.g., compound A1 or A2) administered is sufficient to provide a trough plasma concentration of the compound ranging from about 1 nM to about 500 nM. In another embodiment, the amount of the compound (e.g., compound A1 or A2) administered is sufficient to provide a trough plasma concentration of the compound ranging from about 2 nM to about 200 nM. In yet another embodiment, the amount of the compound (e.g., compound A1 or A2) administered is sufficient to provide a trough plasma concentration of the compound ranging from about 5 nM to about 100 nM. In still another embodiment, the amount of the compound (e.g., compound A1 or A2) administered is sufficient to provide a trough plasma concentration of the compound ranging from about 10 nM to about 50 nM.

In still another embodiment, provided herein is a method for treating, preventing, or ameliorating one or more symptoms of a liver disease or disorder associated with an HCV infection in a subject, comprising administering to the subject the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein; in an amount that is sufficient to provide an area under the curve (AUC) of the compound in the range from about 100 to about 10,000 ng·hr/mL, from about 100 to 5,000 ng·hr/mL, from about 100 to 2,000 ng·hr/mL, from about 200 to 2,000 ng·hr/mL. In one embodiment, the amount of the compound (e.g., compound A1 or A2) administered is sufficient to provide an AUC of the compound in the range from about 100 to about 10,000 ng·hr/mL. In another embodiment, the amount of the compound (e.g., compound A1 or A2) administered is sufficient to provide an AUC of the compound in the range from about 100 to 5,000 ng·hr/mL. In yet another embodiment, the amount of the compound (e.g., compound A1 or A2) administered is sufficient to provide an AUC of the compound in the range from about 100 to 2,000 ng·hr/mL. In still another embodiment, the amount of the compound (e.g., compound A1 or A2) administered is sufficient to provide an AUC of the compound in the range from about 200 to 2,000 ng·hr/mL.

In one embodiment, provided herein is a method for inhibiting replication of a Flaviviridae virus in a subject, comprising administering to the subject a therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein; wherein the therapeutically effective amount is as defined herein.

In another embodiment, provided herein is a method for inhibiting replication of a Flaviviridae virus in a subject, comprising administering to the subject a therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein; wherein the therapeutically effective amount is as defined herein.

In yet another embodiment, provided herein is a method for inhibiting replication of a Flaviviridae virus in a subject, comprising administering to the subject the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein; in an amount that is sufficient to provide a plasma concentration of the compound at steady state in the range from about 1 nM to about 1 M, from about 2 nM to about 500 nM, from about 5 nM to about 200 nM, from about 10 nM to about 100 nM, or from about 10 nM to about 50 nM. In one embodiment, the amount of the compound (e.g., compound A1 or A2) administered is sufficient to provide a plasma concentration of the compound at steady state in the range from about 1 nM to about 1 μM. In another embodiment, the amount of the compound (e.g., compound A1 or A2) administered is sufficient to provide a plasma concentration of the compound at steady state in the range from about 2 nM to about 500 nM. In yet another embodiment, the amount of the compound (e.g., compound A1 or A2) administered is sufficient to provide a plasma concentration of the compound at steady state in the range from about 5 nM to about 200 nM. In yet another embodiment, the amount of the compound (e.g., compound A1 or A2) administered is sufficient to provide a plasma concentration of the compound at steady state in the range from about 10 nM to about 100 nM. In still another embodiment, the amount of the compound (e.g., compound A1 or A2) administered is sufficient to provide a plasma concentration of the compound at steady state in the range from about 10 nM to about 50 nM.

In yet another embodiment, provided herein is a method for inhibiting replication of a Flaviviridae virus in a subject, comprising administering to the subject the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein; in an amount that is sufficient to provide a peak plasma concentration of the compound ranging from about 5 nM to about 1 μM, from about 10 nM to about 500 nM, from about 20 nM to about 200 nM, or from about 50 nM to about 100 nM. In one embodiment, the amount of the compound (e.g., compound A1 or A2) administered is sufficient to provide a peak plasma concentration of the compound ranging from about 5 nM to about 1 μM. In another embodiment, the amount of the compound (e.g., compound A1 or A2)administered is sufficient to provide a peak plasma concentration of the compound ranging from about 10 nM to about 500 nM. In yet another embodiment, the amount of the compound (e.g., compound A1 or A2)administered is sufficient to provide a peak plasma concentration of the compound ranging from about 20 nM to about 200 nM. In still another embodiment, the amount of the compound (e.g., compound A1 or A2) administered is sufficient to provide a peak plasma concentration of the compound ranging from about 50 nM to about 100 nM.

In yet another embodiment, provided herein is a method for inhibiting replication of a Flaviviridae virus in a subject, comprising administering to the subject the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein; in an amount that is sufficient to provide a trough plasma concentration of the compound ranging from about 1 nM to about 500 nM, from about 2 nM to about 200 nM, from about 5 nM to about 100 nM, from about 10 nM to about 50 nM. In one embodiment, the amount of the compound (e.g., compound A1 or A2) administered is sufficient to provide a trough plasma concentration of the compound ranging from about 1 nM to about 500 nM. In another embodiment, the amount of the compound (e.g., compound A1 or A2) administered is sufficient to provide a trough plasma concentration of the compound ranging from about 2 nM to about 200 nM. In yet another embodiment, the amount of the compound (e.g., compound A1 or A2) administered is sufficient to provide a trough plasma concentration of the compound ranging from about 5 nM to about 100 nM. In still another embodiment, the amount of the compound (e.g., compound A1 or A2) administered is sufficient to provide a trough plasma concentration of the compound ranging from about 10 nM to about 50 nM.

In still another embodiment, provided herein is a method for inhibiting replication of a Flaviviridae virus in a subject, comprising administering to the subject the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein; in an amount that is sufficient to provide an area under the curve (AUC) of the compound in the range from about 100 to about 10,000 ng·hr/mL, from about 100 to 5,000 ng·hr/mL, from about 100 to 2,000 ng·hr/mL, from about 200 to 2,000 ng·hr/mL. In one embodiment, the amount of the compound (e.g., compound A1 or A2) administered is sufficient to provide an AUC of the compound in the range from about 100 to about 10,000 ng·hr/mL. In another embodiment, the amount of the compound (e.g., compound A1 or A2) administered is sufficient to provide an AUC of the compound in the range from about 100 to 5,000 ng·hr/mL. In yet another embodiment, the amount of the compound (e.g., compound A1 or A2) administered is sufficient to provide an AUC of the compound in the range from about 100 to 2,000 ng·hr/mL. In still another embodiment, the amount of the compound (e.g., compound A1 or A2) administered is sufficient to provide an AUC of the compound in the range from about 200 to 2,000 ng·hr/mL.

In certain embodiments, the Flaviviridae virus is a pestivirus. In certain embodiments, the Flaviviridae virus is a flavivirus. In certain embodiments, the Flaviviridae virus is West Nile virus, a dengue hemorrhagic fever viruse, yellow fever virus, Japanese encephalitis virus, or bovine viral diarrhea virus.

In certain embodiments, the Flaviviridae virus is a hepatitis C virus. In certain embodiments, the virus is a drug resistant virus. In certain embodiments, the virus is a drug resistant hepatitis C virus.

In one embodiment, the hepatitis C virus is HCV genotype 1. In certain embodiments, the hepatitis C virus is HCV subtype 1a. In certain embodiments, the hepatitis C virus is HCV subtype 1b. In certain embodiments, the hepatitis C virus is HCV subtype 1c.

In another embodiment, the hepatitis C virus is HCV genotype 2. In certain embodiments, the hepatitis C virus is HCV subtype 2a. In certain embodiments, the hepatitis C virus is HCV subtype 2b. In certain embodiments, the hepatitis C virus is HCV subtype 2c.

In yet another embodiment, the hepatitis C virus is HCV genotype 3. In certain embodiments, the hepatitis C virus is HCV subtype 3a. In certain embodiments, the hepatitis C virus is HCV subtype 3b.

In yet another embodiment, the hepatitis C virus is HCV genotype 4. In certain embodiments, the hepatitis C virus is HCV subtype 4a. In certain embodiments, the hepatitis C virus is HCV subtype 4b. In certain embodiments, the hepatitis C virus is HCV subtype 4c. In certain embodiments, the hepatitis C virus is HCV subtype 4d. In certain embodiments, the hepatitis C virus is HCV subtype 4e.

In yet another embodiment, the hepatitis C virus is HCV genotype 5. In yet another embodiment, the hepatitis C virus is HCV subtype 5a.

In yet another embodiment, the hepatitis C virus is HCV genotype 6. In yet another embodiment, the hepatitis C virus is HCV subtype 6a.

In yet another embodiment, the hepatitis C virus is HCV genotype 7. In yet another embodiment, the hepatitis C virus is HCV subtype 7a.

In yet another embodiment, the hepatitis C virus is HCV genotype 8. In yet another embodiment, the hepatitis C virus is HCV subtype 8a. In yet another embodiment, the hepatitis C virus is HCV subtype 8b.

In yet another embodiment, the hepatitis C virus is HCV genotype 9. In yet another embodiment, the hepatitis C virus is HCV subtype 9a.

In yet another embodiment, the hepatitis C virus is HCV genotype 10. In yet another embodiment, the hepatitis C virus is HCV subtype 10a.

In still another embodiment, the hepatitis C virus is HCV genotype 11. In yet another embodiment, the hepatitis C virus is HCV subtype 11a.

In one embodiment, the HCV is a HCV variant. In another embodiment, the virus is a HCV variant.

In one embodiment, the HCV variant is a variant of HCV genotype 1. In certain embodiments, the HCV variant is a variant of HCV subtype 1a. In certain embodiments, the HCV variant is a variant of HCV subtype 1b. In certain embodiments, the HCV variant is a variant of HCV subtype 1c.

In certain embodiments, the HCV variant is a variant of HCV subtype 1a, which contains an NS5A protein variant. In certain embodiments, the NS5A protein variant contains a mutation or deletion. In certain embodiments, the NS5A protein variant contains one or more mutations and/or deletions at the amino acid positions of 28, 30, 31, 32, 54, and 93. In certain embodiments, the NS5A protein variant contains one or more mutations and/or deletions at the amino acid positions of 23, 24, 28, 30, 31, 32, 37, 54, 58, 63, 93, 295, 318, 320, 356, 404, and 442. In certain embodiments, the NS5A protein variant contains one or more mutations and/or deletions at the amino acid positions of 24, 28, 30, 31, 32, 54, 93, 295, and 318. In certain embodiments, the NS5A protein variant contains one, two, or more mutations and/or deletions, each independently selected from M28T, AQ30, Q30E, Q30H, Q30K, Q30R, L31F, L31M, L31V, P32L, H54Y, Y93C, Y93H, and Y93N, provided that there is only one mutation or deletion at a given amino acid position in the NS5A protein variant. In certain embodiments, the NS5A protein variant contains one, two, or more mutations and/or deletions, each independently selected from L23F, K24E, L28M, L28T, M28T, AQ30, Q30E, Q30H, Q30K, Q30R, AR30, R30E, R30Q, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from L23F, K24E, L28M, L28T, AQ30, Q30E, Q30H, Q30K, Q30R, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from L23F, K24E, M28T, AR30, R30E, R30Q, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from K24E, M28T, Q30E, Q30H, Q30K, Q30R, L31F, L31M, L31V, P32L, Y93C, Y93H, Y93N, E295G, and R318W, provided that there is only one mutation or deletion at a given amino acid position in the NS5A protein variant. In certain embodiments, the NS5A protein variant contains one or more mutations at the amino acid positions of 28, 30, 31, 32, and 93. In certain embodiments, the NS5A protein variant contains one, two, or more mutations, each independently selected from M28T, Q30E, Q30H, Q30K, Q30R, L31F, L31M, L31V, P32L, Y93C, Y93H, and Y93N, provided that there is only one mutation at a given amino acid position in the NS5A protein variant. In certain embodiments, the NS5A protein variant contains one or more mutations at the amino acid positions of 24, 28, 30, 31, 32, 93, 295, and 318. In certain embodiments, the NS5A protein variant contains one, two, or more mutations, each independently selected from K24E, M28T, Q30E, Q30H, Q30K, Q30R, L31F, L31M, L31V, P32L, Y93C, Y93H, Y93N, E295G, and R318W, provided that there is only one mutation at a given amino acid position in the NS5A protein variant.

In certain embodiments, the HCV variant is a variant of HCV subtype 1b, which contains an NS5A protein variant. In certain embodiments, the NS5A protein variant contains a mutation or deletion. In certain embodiments, the NS5A protein variant contains one or more mutations and/or deletions at the amino acid positions of 23, 28, 30, 31, 32, 37, 54, 58, 63, and 93. In certain embodiments, the NS5A protein variant contains one or more mutations and/or deletions at the amino acid positions of 23, 24, 28, 30, 31, 32, 37, 54, 58, 63, 93, 295, 318, 320, 356, 404, and 442. In certain embodiments, the NS5A protein variant contains one or more mutations and/or deletions at the amino acid positions of 24, 28, 30, 31, 32, 54, 93, 295, and 318. In certain embodiments, the NS5A protein variant contains one, two, or more mutations and/or deletions, each independently selected from L23F, L28M, L28T, AR30, R30E, R30Q, L31F, L31M, L31V, P32L, F37L, Q54H, P58H, P58S, I63V, Y93C, Y93H, Y93N, and Y93S, provided that there is only one mutation or deletion at a given amino acid position in the NS5A protein variant. In certain embodiments, the NS5A protein variant contains one, two, or more mutations and/or deletions, each independently selected from L23F, K24E, L28M, L28T, M28T, AQ30, Q30E, Q30H, Q30K, Q30R, AR30, R30E, R30Q, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from L23F, K24E, L28M, L28T, AQ30, Q30E, Q30H, Q30K, Q30R, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from L23F, K24E, M28T, AR30, R30E, R30Q, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from K24E, M28T, Q30E, Q30H, Q30K, Q30R, L31F, L31M, L31V, P32L, Y93C, Y93H, Y93N, E295G, and R318W, provided that there is only one mutation or deletion at a given amino acid position in the NS5A protein variant. In certain embodiments, the NS5A protein variant contains one or more mutations at the amino acid positions of 28, 30, 31, 32, and 93. In certain embodiments, the NS5A protein variant contains one, two, or more mutations, each independently selected from L28T, R30E, L31F, L31M, L31V, P32L, Y93C, Y93H, and Y93N, provided that there is only one mutation at a given amino acid position in the NS5A protein variant. In certain embodiments, the NS5A protein variant contains one or more mutations at the amino acid positions of 24, 28, 30, 31, 32, 93, 295, and 318. In certain embodiments, the NS5A protein variant contains one, two, or more mutations, each independently selected from K24E, M28T, Q30E, Q30H, Q30K, Q30R, L31F, L31M, L31V, P32L, Y93C, Y93H, Y93N, E295G, and R318W, provided that there is only one mutation at a given amino acid position in the NS5A protein variant.

In another embodiment, the HCV variant is a variant of HCV genotype 2. In certain embodiments, the HCV variant is a variant of HCV subtype 2a. In certain embodiments, the HCV variant is a variant of HCV subtype 2b. In certain embodiments, the HCV variant is a variant of HCV subtype 2c.

In yet another embodiment, the HCV variant is a variant of HCV genotype 3. In certain embodiments, the HCV variant is a variant of HCV subtype 3a. In certain embodiments, the HCV variant is a variant of HCV subtype 3b.

In yet another embodiment, the HCV variant is a variant of HCV genotype 4. In certain embodiments, the HCV variant is a variant of HCV subtype 4a. In certain embodiments, the HCV variant is a variant of HCV subtype 4b. In certain embodiments, the HCV variant is a variant of HCV subtype 4c. In certain embodiments, the HCV variant is a variant of HCV subtype 4d. In certain embodiments, the HCV variant is a variant of HCV subtype 4e.

In yet another embodiment, the HCV variant is a variant of HCV genotype 5. In yet another embodiment, the HCV variant is a variant of HCV subtype 5a.

In yet another embodiment, the HCV variant is a variant of HCV genotype 6. In yet another embodiment, the HCV variant is a variant of HCV subtype 6a.

In yet another embodiment, the HCV variant is a variant of HCV genotype 7. In yet another embodiment, the HCV variant is a variant of HCV subtype 7a.

In yet another embodiment, the HCV variant is a variant of HCV genotype 8. In yet another embodiment, the HCV variant is a variant of HCV subtype 8a. In yet another embodiment, the HCV variant is a variant of HCV subtype 8b.

In yet another embodiment, the HCV variant is a variant of HCV genotype 9. In yet another embodiment, the HCV variant is a variant of HCV subtype 9a.

In yet another embodiment, the HCV variant is a variant of HCV genotype 10. In yet another embodiment, the HCV variant is a variant of HCV subtype 10a.

In still another embodiment, the HCV variant is a variant of HCV genotype 11. In yet another embodiment, the HCV variant is a variant of HCV subtype 11a.

In certain embodiments, administration of a therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, results in a 90%, 99%, or 99.9% reduction in the replication of the virus relative to a subject without administration of the compound, as determined at 1 day, 2 days, 3 days, 4 days, 5 days, 10 days, 15 days, or 30 days after the administration by a method known in the art, e.g., determination of viral titer. In certain embodiments, the administration of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, results in a 90% reduction in the replication of the virus relative to a subject without administration of the compound, as determined at 1 day after the administration. In certain embodiments, the administration of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, results in a 99% reduction in the replication of the virus relative to a subject without administration of the compound, as determined at 1 day after the administration. In certain embodiments, the administration of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, results in a 99.9% reduction in the replication of the virus relative to a subject without administration of the compound, as determined at 1 day after the administration.

In certain embodiments, administration of a therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, results in a 10-fold (1 log₁₀), 100-fold (2 log₁₀), 1,000-fold (3 log₁₀), or 10,000-fold (4 log₁₀) reduction in the replication of the virus relative to a subject without administration of the compound, as determined at 1 day, 2 days, 3 days, 4 days, 5 days, 10 days, 15 days, or 30 days after the administration by a method known in the art. In certain embodiments, the administration of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, results in a 1 log₁₀ reduction in the replication of the virus relative to a subject without administration of the compound, as determined at 1 day after the administration. In certain embodiments, the administration of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, results in a 2 log₁₀ reduction in the replication of the virus relative to a subject without administration of the compound, as determined at 1 day after the administration. In certain embodiments, the administration of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, results in a 3 log₁₀ reduction in the replication of the virus relative to a subject without administration of the compound, as determined at 1 day after the administration. In certain embodiments, the administration of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, results in a 4 log₁₀ reduction in the replication of the virus relative to a subject without administration of the compound, as determined at 1 day after the administration.

In certain embodiments, administration of a therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, results in a 90%, 99%, or 99.9% reduction in the viral titer relative to a subject without administration of the compound, as determined at 1 day, 2 days, 3 days, 4 days, 5 days, 10 days, 15 days, or 30 days after the administration by a method known in the art. In certain embodiments, the administration of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, results in a 90% reduction in the viral titer relative to a subject without administration of the compound, as determined at 1 day after the administration. In certain embodiments, the administration of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, results in a 99% reduction in the viral titer relative to a subject without administration of the compound, as determined at 1 day after the administration. In certain embodiments, the administration of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, results in a 99.9% reduction in the viral titer relative to a subject without administration of the compound, as determined at 1 day after the administration.

In certain embodiments, administration of a therapeutically effective amount of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, results in a 10-fold (1 log₁₀), 100-fold (2 log₁₀), 1,000-fold (3 log₁₀), or 10,000-fold (4 log₁₀) in the viral titer relative to a subject without administration of the compound A1 or A2, as determined at 1 day, 2 days, 3 days, 4 days, 5 days, 10 days, 15 days, or 30 days after the administration by a method known in the art. In certain embodiments, the administration of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, results in a 1 log₁₀ reduction in the viral titer relative to a subject without administration of the compound, as determined at 1 day after the administration. In certain embodiments, the administration of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, results in a 2 log₁₀ reduction in the viral titer relative to a subject without administration of the compound, as determined at 1 day after the administration. In certain embodiments, the administration of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, results in a 3 log₁₀ reduction in the viral titer relative to a subject without administration of the compound, as determined at 1 day after the administration. In certain embodiments, the administration of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, results in a 4 log₁₀ reduction in the viral titer relative to a subject without administration of the compound, as determined at 1 day after the administration.

In certain embodiments, the subject to be treated with one of the methods provided herein has not been treated with anti-HCV therapy (i.e., treatment-naive) prior to the administration of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein. In certain embodiments, the subject to be treated with one of the methods provided herein has been treated with anti-HCV therapy prior to the administration of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein. In certain embodiments, the subject to be treated with one of the methods provided herein has not been treated with an NS5A inhibitor prior to the administration of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein. In certain embodiments, the subject to be treated with one of the methods provided herein has been treated with an NS5A inhibitor prior to the administration of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein.

In certain embodiments, the subject is a human.

In certain embodiments, the subject has an IL28B (interleukin 28B) CC genotype. In certain embodiments, the subject has an IL28B CT genotype. In certain embodiments, the subject has an IL28B TT genotype.

The methods provided herein encompass treating a subject regardless of patient's age, although some diseases or disorders are more common in certain age groups.

Depending on the disease to be treated and the subject's condition, the compound provided herein (e.g., compound A1 or A2), or an isotopic variant thereof, or a pharmaceutically acceptable salt thereof, or pharmaceutically acceptable solvate thereof, may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. The compound provided herein (e.g., compound A1 or A2), or an isotopic variant thereof, or a pharmaceutically acceptable salt thereof, or pharmaceutically acceptable solvate thereof, may be formulated, alone or together, in suitable dosage unit with pharmaceutically acceptable excipients, carriers, adjuvants and vehicles, appropriate for each route of administration. In one embodiment, the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is administered orally. In another embodiment, the compound provided herein (e.g., compound A1 or A2), or an isotopic variant thereof, or a pharmaceutically acceptable salt thereof, or pharmaceutically acceptable solvate thereof, is administered parenterally. In yet another embodiment, the compound provided herein (e.g., compound A1 or A2), or an isotopic variant thereof, or a pharmaceutically acceptable salt thereof, or pharmaceutically acceptable solvate thereof, is administered intravenously.

The compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, can be delivered as a single dose, such as, e.g., a single bolus injection, or a single oral tablet or pill; or over time, such as, e.g., continuous infusion over time or divided bolus doses over time. The compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, can be administered repetitively if necessary, for example, until the patient experiences stable disease or regression, or until the patient experiences disease progression or unacceptable toxicity. Stable disease or lack thereof is determined by methods known in the art such as evaluation of patient's symptoms, physical examination, or measuring patient's viral level.

The compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, can be administered once daily (QD), or divided into multiple daily doses, such as twice daily (BID), three times daily (TID), and four times daily (QID).

In certain embodiments, The compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is administered once daily (QD). In certain embodiments, The compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is administered twice a day (BID). In certain embodiments, The compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is administered three times a day (TID). In certain embodiments, The compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is administered four times a day (QID).

In certain embodiments, The compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is administered on an empty stomach. In certain embodiments, The compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is administered at least about one hour before eating or at least about two hours after eating. In certain embodiments, The compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is administered at least about one hour before eating. In certain embodiments, The compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is administered at least about two hours after eating.

In certain embodiments, The compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is administered with the help of a chaser. In certain embodiments, the chase is a beverage. In certain embodiments, the chase is a beverage having a low pH. In certain embodiments, the chase is a beverage having a pH of no greater than about 5, no greater than about 4, no greater than about 3, or no greater than about 2. In certain embodiments, the chase is a beverage having a pH of no greater than about 3. In certain embodiments, the chase is a beverage having a pH between about 2 to about 3. In certain embodiments, the chase is a caffeine-free beverage. In certain embodiments, the chase is COCA COLA®. In certain embodiments, the chase is caffeine-free COCA COLA®. In certain embodiments, the chase is regular COCA COLA®. In certain embodiments, the chase is regular caffeine-free COCA COLA®.

### Combination Therapy

The compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, may also be combined or used in combination with other therapeutic agents useful in the treatment and/or prevention of an HCV infection.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

As used herein, the term “synergistic” includes a combination of a compound provided herein and another therapy (e.g., a prophylactic or therapeutic agent) which has been or is currently being used to prevent, treat, or manage a condition, disorder, or disease, which is more effective than the additive effects of the therapies. A synergistic effect of a combination of therapies (e.g., a combination of prophylactic or therapeutic agents) permits the use of lower dosages of one or more of the therapies and/or less frequent administration of said therapies to a subject with a condition, disorder, or disease. The ability to utilize lower dosages of a therapy (e.g., a prophylactic or therapeutic agent) and/or to administer said therapy less frequently reduces the toxicity associated with the administration of said therapy to a subject without reducing the efficacy of said therapy in the prevention, treatment, or management of a condition, disorder, or disease). In addition, a synergistic effect can result in improved efficacy of agents in the prevention, treatment, or management of a condition, disorder, or disease. Finally, a synergistic effect of a combination of therapies (e.g., a combination of prophylactic or therapeutic agents) may avoid or reduce adverse or unwanted side effects associated with the use of either therapy alone.

The compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, can be administered in combination or alternation with another therapeutic agent, such as an anti-HCV agent. In combination therapy, effective dosages of two or more agents are administered together, whereas in alternation or sequential-step therapy, an effective dosage of each agent is administered serially or sequentially. The dosages given will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

It has been recognized that drug-resistant variants of HCV can emerge after prolonged treatment with an antiviral agent. Drug resistance most typically occurs due to the mutation of a gene that encodes for an enzyme used in viral replication. The efficacy of a drug against the viral infection can be prolonged, augmented, or restored by of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, in combination or alternation with a second, and perhaps third, antiviral compound that induces a different mutation from that caused by the principle drug. Alternatively, the pharmacokinetics, biodistribution, or other parameters of the drug can be altered by such combination or alternation therapy. In general, combination therapy is typically preferred over alternation therapy because it induces multiple simultaneous stresses on the virus.

In certain embodiments, the pharmaceutical compositions provided herein further comprise a second antiviral agent (i.e., an antiviral agent other than compound A1 or A2) as described herein. In certain embodiments, the second antiviral agent is a single chemical entity. In certain embodiments, the second antiviral agent is a mixture of two or more chemical entities. In certain embodiments, the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is combined with one or more agents selected from the group an interferon, ribavirin, amantadine, an interleukin, an NS3 protease inhibitor, an NS5A inhibitor, an NS5B inhibitor, a cyclophilin inhibitor, a cysteine protease inhibitor, a phenanthrenequinone, a thiazolidine, a benzanilide, a helicase inhibitor, a polymerase inhibitor, a nucleotide analogue, a nucleoside analogue, a gliotoxin, a cerulenin, an antisense phosphorothioate oligodeoxynucleotide, an inhibitor of IRES-dependent translation, and a ribozyme. In one embodiment, the second antiviral agent is an interferon. In another embodiment, the interferon is selected from pegylated interferon alpha 2a, interferon alfacon-1, natural interferon, ALBUFERON®, interferon beta-1a, omega interferon, interferon alpha, interferon gamma, interferon tau, interferon delta, and interferon gamma-1b.

In certain embodiments, the pharmaceutical compositions provided herein further comprise a cyclophilin inhibitor, including, but not limited to, alisporivir (Novartis), cyclosporin A, sanglifehrins and sanglifehrin analogs, CsD, NIM-811, and SCY-635.

In certain embodiments, the pharmaceutical composition provided herein further comprises an NS5A inhibitor, including, but not limited to, ABT-267, BMS-790052, GS-5885, GS-5816, PPI-461, and PPI-668.

In certain embodiments, a pharmaceutical composition comprising a crystalline or salt form of the compound of Formula I, or a pharmaceutically acceptable solvate or prodrug thereof, is combined with an NS5B inhibitor, including, but not limited to, ABT-072, ABT-333, ANA598, BI 207127, GS-9669, GS-9190, GSK-625433, HCV-796, IDX184, IDX375, IDX19368, IDX437, JTK-109, MK-0608, MK-3281, NM283, PF-868554, PSI-879, PSI-938, PSI-6130, PSI-7851, PSI-7977, R1626, R7128, VCH-222, VCH-759, and VCH-916. In one embodiment, the NS5B inhibitor is PSI-7977 (sofosbuvir). In another embodiment, the NS5B inhibitor is IDX437 (see, e.g., co-pending U.S. provisional patent application (atty. dkt. IDX 1150), filed Mar. 5, 2014).

In certain embodiments, of the compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, is combined with an HCV protease inhibitor, including, but not limited to, ACH-0141625, faldaprevir (BI 201335); asunaprevir (BMS-650032); TMC 435 or TMC 435350 (Medivir/Tibotec); ITMN 191/R7227 (InterMune); MK 7009 (Merck); SCH 5034/SCH 503034/Boceprevir and SCH 900518/narlaprevir (Schering); VX950/telaprevir (Vertex); substrate-based NS3 protease inhibitors as disclosed in DE 19914474, WO 98/17679, WO 98/22496, WO 99/07734, and Attwood et al., Antiviral Chemistry and Chemotherapy 1999, 10, 259-273; non-substrate-based NS3 protease inhibitors, including 2,4,6-trihydroxy-3-nitro-benzamide derivatives (Sudo et al., Biochem. Biophys. Res. Commun. 1997, 238, 643-647), a phenanthrenequinone (Chu et al., Tetrahedron Letters 1996, 37, 7229-7232), RD3-4082, RD3-4078, SCH 68631, and SCH 351633 (Chu et al., Bioorganic and Medicinal Chemistry Letters 1999, 9, 1949-1952); and Eglin C, a potent serine protease inhibitor (Qasim et al., Biochemistry 1997, 36, 1598-1607).

Other suitable protease inhibitors for the treatment of HCV include those disclosed in, for example, U.S. Pat. No. 6,004,933, which discloses a class of cysteine protease inhibitors of HCV endopeptidase 2.

Additional hepatitis C virus NS3 protease inhibitors include those disclosed in, for example, Llinas-Brunet et al., Bioorg. Med. Chem. Lett. 1998, 8, 1713-1718; Steinkiihler et al., Biochemistry 1998, 37, 8899-8905; U.S. Pat. Nos. 5,538,865; 5,990,276; 6,143,715; 6,265,380; 6,323,180; 6,329,379; 6,410,531; 6,420,380; 6,534,523; 6,608,027; 6,642,204; 6,653,295; 6,727,366; 6,838,475; 6,846,802; 6,867,185; 6,869,964; 6,872,805; 6,878,722; 6,908,901; 6,911,428; 6,995,174; 7,012,066; 7,041,698; 7,091,184; 7,169,760; 7,176,208; 7,208,600; and 7,491,794; U.S. Pat. App. Pub. Nos.: 2002/0016294, 2002/0016442; 2002/0032175; 2002/0037998; 2004/0229777; 2005/0090450; 2005/0153877; 2005/176648; 2006/0046956; 2007/0021330; 2007/0021351; 2007/0049536; 2007/0054842; 2007/0060510; 2007/0060565; 2007/0072809; 2007/0078081; 2007/0078122; 2007/0093414; 2007/0093430; 2007/0099825; 2007/0099929; 2007/0105781, 2008/0152622, 2009/0035271, 2009/0035272, 2009/0047244, 2009/0111969, 2009/0111982, 2009/0123425, 2009/0130059, 2009/0148407, 2009/0156800, 2009/0169510, 2009/0175822, 2009/0180981, and 2009/0202480; U.S. patent application Ser. No. 12/365,127; and International Pat. App. Pub. Nos.: WO 98/17679; WO 98/22496; WO 99/07734; WO 00/09543; WO 00/59929; WO 02/08187; WO 02/08251; WO 02/08256; WO 02/08198; WO 02/48116; WO 02/48157; WO 02/48172; WO 02/60926; WO 03/53349; WO 03/64416; WO 03/64455; WO 03/64456; WO 03/66103; WO 03/99274; WO 03/99316; WO 2004/032827; WO 2004/043339; WO 2005/037214; WO 2005/037860; WO 2006/000085; WO 2006/119061; WO 2006/122188; WO 2007/001406; WO 2007/014925; WO 2007/014926; WO 2007/015824, WO 2007/056120, WO 2008/019289, WO 2008/021960, WO 2008/022006, WO 2008/086161, WO 2009/053828, WO 2009/058856, WO 2009/073713, WO 2009/073780, WO 2009/080542, WO 2009/082701, WO 2009/082697, and WO 2009/085978; the disclosure of each of which is incorporated herein by reference in its entirety.

Other protease inhibitors include thiazolidine derivatives, such as RD-1-6250, RD4 6205, and RD4 6193 (Sudo et al., Antiviral Research 1996, 32, 9-18); and thiazolidines and benzanilides (Kakiuchi et al., FEBS Lett. 1998, 421, 217-220; and Takeshita et al., Analytical Biochemistry 1997, 247, 242-246).

Suitable helicase inhibitors include, but are not limited to, those disclosed in U.S. Pat. No. 5,633,358; and International Pat. App. Pub. No. WO 97/36554.

Suitable nucleotide polymerase inhibitors include, but are not limited to, 2′-methyl ribofuranosyl nucleotides. See, e.g., WO 01/90121, WO 01/92282, WO 2004/002999, WO 2005/003147, U.S. Pat. Nos. 6,914,054; 7,608,597; 7,608,600; 7,824,851; 7,157,441; 7,635,689; 7,429,572; 7,754,699; 7,964,580; 7,105,499; 6,777,395; 8,481,712. In one embodiment, a nucleotide polymerase inhibitor is gliotoxin (Ferrari et al., Journal of Virology 1999, 73, 1649-1654), cerulenin (Lohmann et al., Virology 1998, 249, 108-118), ABT-072, ABT-333, AG-02154, ANA598, ANA773, GS-9190, HCV-796, IDX184, IDX375, IDX437, JTK-109, MK-0608, MK-3281, NM283, PF-868554, PSI-879, PSI-938, PSI-6130, PSI-7851, sofosbuvir (PSI-7977), R1626, R7128, RG7128, VCH-759, VCH-916 or VX-222 (VCH-222).

Suitable interfering RNA (iRNA) based antivirals include, but are not limited to, short interfering RNA (siRNA) based antivirals, such as Sirna-034 and those described in International Pat. App. Pub. Nos. WO/03/070750 and WO 2005/012525, and U.S. Pat. App. Pub. No. 2004/0209831.

Suitable antisense phosphorothioate oligodeoxynucleotides (S-ODN) complementary to sequence stretches in the 5′ non-coding region (NCR) of HCV virus include, but are not limited to those described in Alt et al., Hepatology 1995, 22, 707-717, and nucleotides 326-348 comprising the 3′ end of the NCR and nucleotides 371-388 located in the core coding region of HCV RNA (Alt et al., Archives of Virology 1997, 142, 589-599; and Galderisi et al., Journal of Cellular Physiology 1999, 181, 251-257).

Suitable inhibitors of IRES-dependent translation include, but are not limited to, those described in Japanese Pat. App. Pub. Nos.: JP 08268890 and JP 10101591.

Suitable ribozymes include those disclosed in, for example, U.S. Pat. Nos. 6,043,077; 5,869,253; and 5,610,054.

Suitable nucleoside analogs include, but are not limited to, the compounds described in U.S. Pat. Nos. 6,660,721; 6,777,395; 6,784,166; 6,846,810; 6,927,291; 7,094,770; 7,105,499; 7,125,855; and 7,202,224; U.S. Pat. App. Pub. Nos. 2004/0121980; 2005/0009737; 2005/0038240; and 2006/0040890; and International Pat. App. Pub. Nos: WO 99/43691; WO 01/32153; WO 01/60315; WO 01/79246; WO 01/90121, WO 01/92282, WO 02/18404; WO 02/32920, WO 02/48165, WO 02/057425; WO 02/057287; WO 2004/002422, WO 2004/002999, and WO 2004/003000.

Other miscellaneous compounds that can be used as second agents include, for example, 1-amino-alkylcyclohexanes (U.S. Pat. No. 6,034,134), alkyl lipids (U.S. Pat. No. 5,922,757), vitamin E and other antioxidants (U.S. Pat. No. 5,922,757), squalene, amantadine, bile acids (U.S. Pat. No. 5,846,964), N-(phosphonacetyl)-L-aspartic acid (U.S. Pat. No. 5,830,905), benzenedicarboxamides (U.S. Pat. No. 5,633,388), polyadenylic acid derivatives (U.S. Pat. No. 5,496,546), 2′,3′-dideoxyinosine (U.S. Pat. No. 5,026,687), benzimidazoles (U.S. Pat. No. 5,891,874), plant extracts (U.S. Pat. Nos. 5,725,859; 5,837,257; and 6,056,961), and piperidines (U.S. Pat. No. 5,830,905).

In certain embodiments, one or more compounds provided herein are administered in combination or alternation with an anti-hepatitis C virus interferon, including, but not limited to, INTRON® A (interferon alfa-2b), PEGASYS® (Peginterferon alfa-2a) ROFERON® A (recombinant interferon alfa-2a), INFERGEN® (interferon alfacon-1), and PEG-INTRON® (pegylated interferon alfa-2b). In one embodiment, the anti-hepatitis C virus interferon is INFERGEN®, IL-29 (PEG-Interferon lambda), R7025 (Maxy-alpha), BELEROFON®, oral interferon alpha, BLX-883 (LOCTERON®), omega interferon, MULTIFERON®, medusa interferon, ALBUFERON®, or REBIF®.

In certain embodiments, one or more compounds provided herein are administered in combination or alternation with an anti-hepatitis C virus polymerase inhibitor, such as ribavirin, viramidine, NM 283 (valopicitabine), ABT-072, ABT-267, ABT-333, AG-02154, ANA598, ANA773, EDP-239, deleobuvir (BI 207127), GS-9190, HCV-796, IDX184, IDX375, JTK-109, MK-0608, MK-3281, NM283, PF-868554, PSI-879, PSI-938, PSI-6130, PSI-7851, sofosbuvir (PSI-7977), R1626, HCV-796, R7128, RG7128, VCH-759, VCH-916, VX-222 (VCH-222), and those as disclosed in U.S. Pat. App. Pub. Nos. 2009/0081158 and 2009/0238790, the disclosure of each of which is incorporated herein by reference in its entirety.

In certain embodiments, one or more compounds provided herein are administered in combination with ribavirin and an anti-hepatitis C virus interferon, such as INTRON® A (interferon alfa-2b), PEGASYS® (Peginterferon alfa-2a), ROFERON® A (recombinant interferon alfa-2a), INFERGEN® (interferon alfacon-1), and PEG-INTRON® (pegylated interferon alfa-2b).

In certain embodiments, one or more compounds provided herein are administered in combination or alternation with an anti-hepatitis C virus protease inhibitor, such as ABT-450, ITMN-191, SCH 503034, VX950 (telaprevir), and TMC 435.

In certain embodiments, one or more compounds provided herein are administered in combination or alternation with an anti-hepatitis C virus vaccine, including, but not limited to, TG4040, PEVIPRO™, CGI-5005, HCV/MF59, GV1001, IC41, and INNOO101 (E1).

In certain embodiments, one or more compounds provided herein are administered in combination or alternation with an anti-hepatitis C virus monoclonal antibody, such as AB68 and XTL-6865 (formerly HepX-C); or an anti-hepatitis C virus polyclonal antibody, such as CIVACIR®.

In certain embodiments, one or more compounds provided herein are administered in combination or alternation with an anti-hepatitis C virus immunomodulator, such as ZADAXIN® (thymalfasin), NOV-205, and oglufanide.

In certain embodiments, one or more compounds provided herein are administered in combination or alternation with NEXAVAR®, doxorubicin, PI-88, amantadine, JBK-122, VGX-410C, MX-3253 (celgosivir), SUVUS® (BIVN-401 or virostat), PF-03491390 (formerly IDN-6556), G126270, UT-231B, DEBIO-025, EMZ702, ACH-0137171, MitoQ, ANA975, AVI-4065, bavituximab (tarvacin), ALINIA® (nitrazoxanide), and PYN17.

The compounds provided herein can also be administered in combination with other classes of compounds, including, but not limited to, (1) alpha-adrenergic agents; (2) antiarrhythmic agents; (3) anti-atherosclerotic agents, such as ACAT inhibitors; (4) antibiotics, such as anthracyclines, bleomycins, mitomycin, dactinomycin, and plicamycin; (5) anticancer agents and cytotoxic agents, e.g., alkylating agents, such as nitrogen mustards, alkyl sulfonates, nitrosoureas, ethylenimines, and triazenes; (6) anticoagulants, such as acenocoumarol, argatroban, bivalirudin, lepirudin, fondaparinux, heparin, phenindione, warfarin, and ximelagatran; (7) anti-diabetic agents, such as biguanides (e.g., metformin), glucosidase inhibitors (e.g., acarbose), insulins, meglitinides (e.g., repaglinide), sulfonylureas (e.g., glimepiride, glyburide, and glipizide), thiozolidinediones (e.g., troglitazone, rosiglitazone, and pioglitazone), and PPAR-gamma agonists; (8) antifungal agents, such as amorolfine, amphotericin B, anidulafungin, bifonazole, butenafine, butoconazole, caspofungin, ciclopirox, clotrimazole, econazole, fenticonazole, filipin, fluconazole, isoconazole, itraconazole, ketoconazole, micafungin, miconazole, naftifine, natamycin, nystatin, oxyconazole, ravuconazole, posaconazole, rimocidin, sertaconazole, sulconazole, terbinafine, terconazole, tioconazole, and voriconazole; (9) antiinflammatories, e.g., non-steroidal anti-inflammatory agents, such as aceclofenac, acemetacin, amoxiprin, aspirin, azapropazone, benorilate, bromfenac, carprofen, celecoxib, choline magnesium salicylate, diclofenac, diflunisal, etodolac, etoricoxib, faislamine, fenbufen, fenoprofen, flurbiprofen, ibuprofen, indometacin, ketoprofen, ketorolac, lornoxicam, loxoprofen, lumiracoxib, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinpyrazone, suprofen, tenoxicam, tiaprofenic acid, and tolmetin; (10) antimetabolites, such as folate antagonists, purine analogues, and pyrimidine analogues; (11) anti-platelet agents, such as GPIIb/IIIa blockers (e.g., abciximab, eptifibatide, and tirofiban), P2Y(AC) antagonists (e.g., clopidogrel, ticlopidine and CS-747), cilostazol, dipyridamole, and aspirin; (12) antiproliferatives, such as methotrexate, FK506 (tacrolimus), and mycophenolate mofetil; (13) anti-TNF antibodies or soluble TNF receptor, such as etanercept, rapamycin, and leflunimide; (14) aP2 inhibitors; (15) beta-adrenergic agents, such as carvedilol and metoprolol; (16) bile acid sequestrants, such as questran; (17) calcium channel blockers, such as amlodipine besylate; (18) chemotherapeutic agents; (19) cyclooxygenase-2 (COX-2) inhibitors, such as celecoxib and rofecoxib; (20) cyclosporins; (21) cytotoxic drugs, such as azathioprine and cyclophosphamide; (22) diuretics, such as chlorothiazide, hydrochlorothiazide, flumethiazide, hydroflumethiazide, bendroflumethiazide, methylchlorothiazide, trichloromethiazide, polythiazide, benzothiazide, ethacrynic acid, ticrynafen, chlorthalidone, furosenide, muzolimine, bumetanide, triamterene, amiloride, and spironolactone; (23) endothelin converting enzyme (ECE) inhibitors, such as phosphoramidon; (24) enzymes, such as L-asparaginase; (25) Factor VIIa Inhibitors and Factor Xa Inhibitors; (26) famesyl-protein transferase inhibitors; (27) fibrates; (28) growth factor inhibitors, such as modulators of PDGF activity; (29) growth hormone secretagogues; (30) HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, atavastatin, or visastatin); neutral endopeptidase (NEP) inhibitors; (31) hormonal agents, such as glucocorticoids (e.g., cortisone), estrogens/antiestrogens, androgens/antiandrogens, progestins, and luteinizing hormone-releasing hormone antagonists, and octreotide acetate; (32) immunosuppressants; (33) mineralocorticoid receptor antagonists, such as spironolactone and eplerenone; (34) microtubule-disruptor agents, such as ecteinascidins; (35) microtubule-stabilizing agents, such as pacitaxel, docetaxel, and epothilones A-F; (36) MTP Inhibitors; (37) niacin; (38) phosphodiesterase inhibitors, such as PDE III inhibitors (e.g., cilostazol) and PDE V inhibitors (e.g., sildenafil, tadalafil, and vardenafil); (39) plant-derived products, such as vinca alkaloids, epipodophyllotoxins, and taxanes; (40) platelet activating factor (PAF) antagonists; (41) platinum coordination complexes, such as cisplatin, satraplatin, and carboplatin; (42) potassium channel openers; (43) prenyl-protein transferase inhibitors; (44) protein tyrosine kinase inhibitors; (45) renin inhibitors; (46) squalene synthetase inhibitors; (47) steroids, such as aldosterone, beclometasone, betamethasone, deoxycorticosterone acetate, fludrocortisone, hydrocortisone (cortisol), prednisolone, prednisone, methylprednisolone, dexamethasone, and triamcinolone; (48) TNF-alpha inhibitors, such as tenidap; (49) thrombin inhibitors, such as hirudin; (50) thrombolytic agents, such as anistreplase, reteplase, tenecteplase, tissue plasminogen activator (tPA), recombinant tPA, streptokinase, urokinase, prourokinase, and anisoylated plasminogen streptokinase activator complex (APSAC); (51) thromboxane receptor antagonists, such as ifetroban; (52) topoisomerase inhibitors; (53) vasopeptidase inhibitors (dual NEP-ACE inhibitors), such as omapatrilat and gemopatrilat; and (54) other miscellaneous agents, such as, hydroxyurea, procarbazine, mitotane, hexamethylmelamine, and gold compounds.

The compound provided herein (e.g., compound A1 or A2), or an isotopic variant thereof, or a pharmaceutically acceptable salt thereof, or pharmaceutically acceptable solvate thereof, in the form of spray-dried particles or a pharmaceutical composition (e.g., a granular formulation, a tablet formulation, or a capsule formulation) provided herein, can also be provided as an article of manufacture using packaging materials well known to those of skill in the art. See, e.g., U.S. Pat. Nos. 5,323,907; 5,052,558; and 5,033,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

Provided herein also are kits which, when used by the medical practitioner, can simplify the administration of appropriate amounts of active ingredients to a subject. In certain embodiments, the kit provided herein includes a container and a dosage form of a compound provided herein, including a single enantiomer, a racemic mixture, a mixture of diastereomers, or an isotopic variant thereof; or a pharmaceutically acceptable salt, solvate, or prodrug thereof.

In certain embodiments, the kit includes a container comprising a dosage form of the compounds provided herein, including a single enantiomer, a racemic mixture, a mixture of diastereomers, or an isotopic variant thereof; or a pharmaceutically acceptable salt, solvate, or prodrug thereof, in a container comprising one or more other therapeutic agent(s) described herein.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

The disclosure will be further understood by the following non-limiting examples.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: kg (kilograms); g (grams); mg (milligrams); μg (micrograms); ng (nanograms); L (liter); mL (milliliters); μL (microliters); mM (millimolar); μM (micromolar); nM (nanomolar); pM (picomolar); hr(s) (hour(s)); min (minute(s)); N (Newton); kN (kiloNewton); kp (kilopond); AUC (area under curve); Cₘₐₓ (maximum concentration); GB (gastric buffer); IB (intestinal buffer); FaSSGF (fasted state simulated gastric fluid); FaSSIF (fasted state simulated intestinal fluid); PBS (phosphate buffered saline); THF (tetrahydrofuran); HPMC (hydroxypropyl methyl cellulose, hydroxypropyl methylcellulose, or hypromellose); HPMCAS (hydroxypropyl methylcellulose acetate succinate or hypromellose acetate succinate); HPMCP (hydroxypropyl methylcellulose phthalate or hypromellose phthalate); PEG (polyethylene glycol); PVP (polyvinyl pyrrolidone, polyvinylpyrrolidone, polyvidone, or povidone); TPGS (tocopherol polyethylene glycol succinate); SEM (scanning electron microscopy); HPLC (high performance liquid chromatography); and RH (relative humidity).

Compound A1 has a low aqueous solubility and low permeability and, thus, is classified as a BCS class IV compound, which presents a significant formulation challenge. Cook et al., AAPS J. 2008, 10, 306-310.

Initial pharmacokinetic studies on compound A1 were performed both in vitro and in the monkey using a solution formulation containing PEG200. Compound A1 in the PEG200 solution was determined to have bioavailability of 0.1% F when administered at a dose of 500 mg/kg to the monkey under fed conditions.

### Example 1

### Preparation of a Spray-Dried Dispersion of Compound A1 from a THF/Methanol Solution

A spray dried dispersion of amorphous compound A1 comprising 25% by weight of compound A1 and 75% by weight of polyvinyl pyrrolidone (PVP-K30) was obtained by spraying a solution containing 5% by weight of compound A1, 15% by weight of PVP-K30, 64% by weight of THF, and 16% by weight of methanol.

Briefly, to a stainless-steel solution tank equipped with an agitator were added THF (3,463 g) and methanol (866.2 g). At a temperature between 15 and 27° C., compound A1 (227 g) was added to the solvent mixture. The resulting mixture was then mixed for 1 hr, followed by the addition of PVP-K30 (856.4 g). The mixture was stirred for an additional hour at the same temperature range to form a spray solution. The resulting solution was spray-dried using GEA-Niro Mobile Minor Spray Dryer at a feed rate of 150-180 g/min, a feed pressure of 425 psig, a drying gas flow rate of 1,800 g/min, a drying gas inlet temperature of 112° C., and a drying gas outlet temperature of 45° C. The spray-dried dispersion was further dried in a tray drier between 40 and 50° C. for at least 16 hrs.

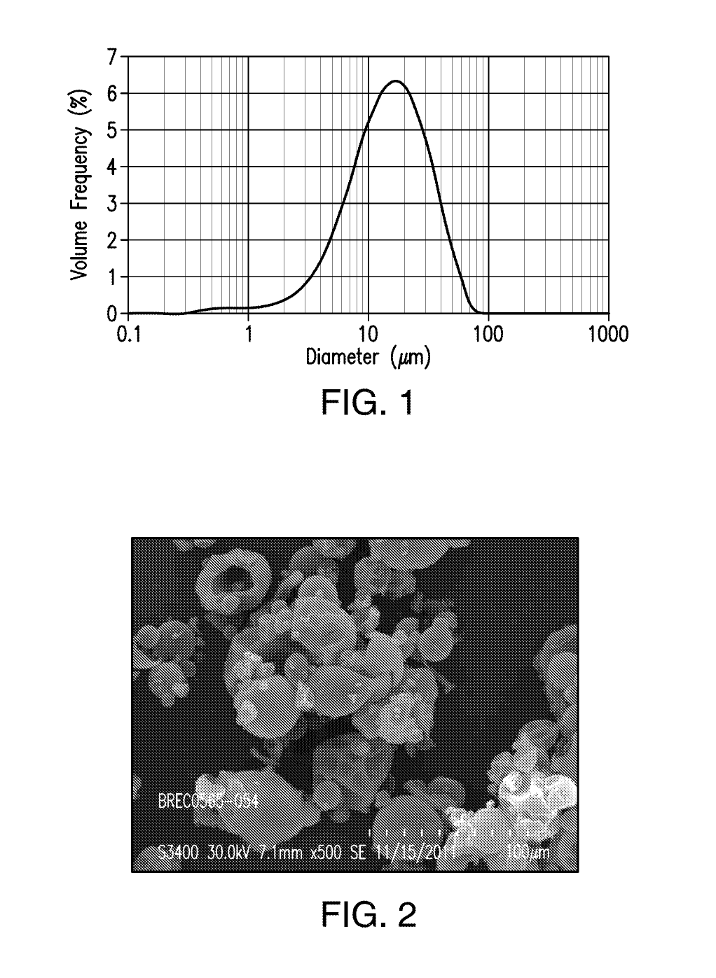

The residual water content of the spray dried dispersion was determined to be 5.7% by weight using the Karl Fischer method. The residual organic solvent content of the spray dried dispersion was analyzed using headspace gas chromatography. The residual methanol content of the spray dried dispersion was determined to be below the limit of quantitation (0.01% by weight). The residual THF content of the spray dried dispersion was determined to be below the limit of quantitation (0.02% by weight). The bulk density of the spray dried dispersion was determined to be 0.11 g/mL. The tapped density was determined to be 0.26 g/mL. The particle size distribution of the spray-dried dispersion was determined and is shown in FIG. 1. A SEM image of the spray dried dispersion was also obtained and is shown in FIG. 2. The spray dried dispersion was also determined to be stable at least for 24 months.

### Example 2

### Preparation of a Spray-Dried Dispersion of Compound A1 from a Methanol/Water Solution

A spray dried dispersion of amorphous compound A1 comprising 25% by weight of compound A1 and 75% by weight of polyvinyl pyrrolidone (PVP-K30) was obtained by spraying a solution containing 5% by weight of compound A1, 15% by weight of PVP-K30, 72% by weight of methanol, and 8% by weight of water.

Briefly, to a stainless-steel solution tank equipped with an agitator were added methanol (4,919 g) and water (546.6 g). At a temperature between 15 and 27° C., compound A1 (350 g) was added to the solvent mixture and the mixture was mixed for 30 min, followed by the addition of PVP-K30 (1,056.5 g). The resulting suspension was mixed in a Bematek High Shear Mixer at the same temperature range to form a spray solution. The resulting solution was spray-dried at a feed rate of 33 kg/hr, a feed pressure of 600 psig, a drying gas flow rate of 450 kg/hr, a drying gas inlet temperature of 125° C.; and a drying gas outlet temperature of 55° C. The spray-dried dispersion was further dried in a tray drier between 40 and 50° C. for at least 8 hrs.

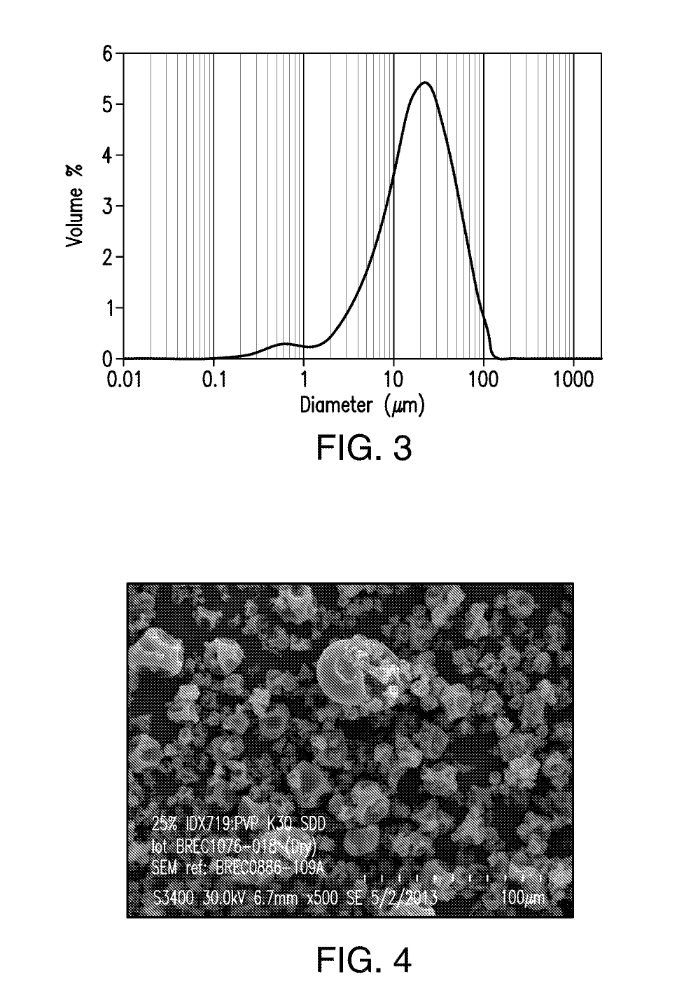

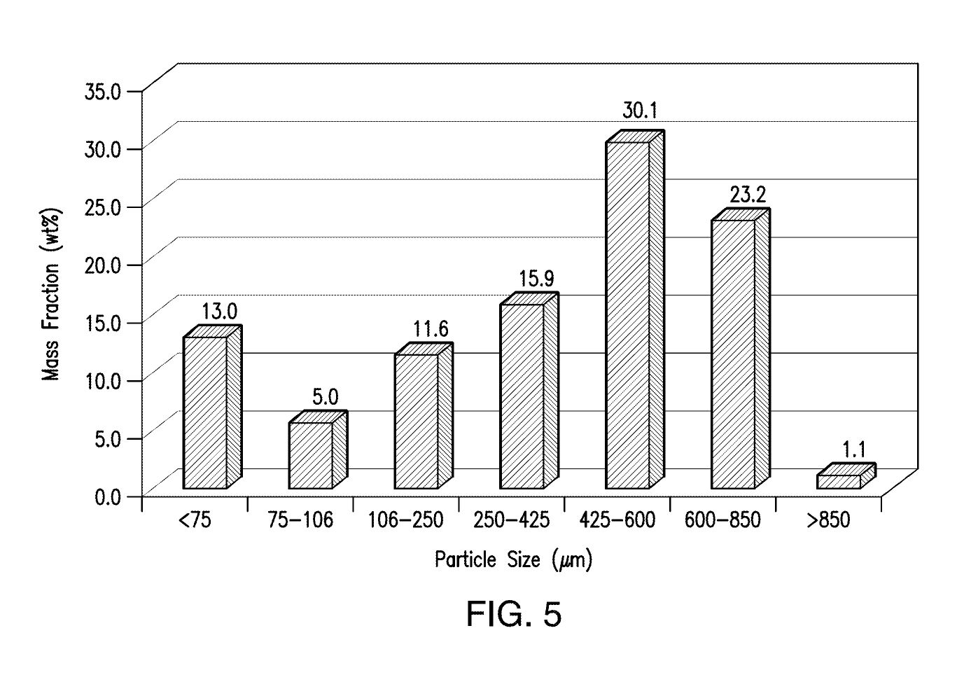

The residual water content of the spray dried dispersion was measured to be 4.5% by weight using the Karl Fischer method. The residual organic solvent content of the spray dried dispersion was analyzed using headspace gas chromatography. The residual methanol content of the spray dried dispersion was determined to be 0.02% by weight. The bulk density of the spray dried dispersion was measured to be 0.25 g/mL. The tapped density of the spray dried dispersion was measured to be 0.53 g/mL. The particle size distribution of the spray-dried dispersion was determined and is shown in FIG. 3. A SEM image of the spray dried dispersion was also obtained and is shown in FIG. 4. The spray dried dispersion was also determined to be stable at least for 6 months.

### Example 3

### Preparation of Pharmaceutical Granules and Tablets

The 25 mg and 50 mg SDD tablets of compound A1 were formulated using an approach in which the compound A1-containing SDD (e.g., SDD made according to Examples 1 and 2) was granulated with a very small number of additives to create large domains of SDD-rich granules embedded in the tablet fillers, which facilitates rapid disintegration of the tablets into the original granule domains.

Compound A1-containing SDD granules were prepared by first blending the Compound A1-containing SDD with intragranular colloidal silicon dioxide (CAB-O-SIL® M-5P) in a blender and the resulting SDD pre-mix was passed through a mill for delumping. After delumped, the SDD-premix was blended with intragranular magnesium stearate. The resulting mixture was then roller-compacted and granulated to form compound A1-containing SDD granules. The ribbon solid fractions, densities, and compressibility of the granules were measured and the results are shown in Table 1.

Compound A1-containing SDD granular tablets were prepared by blending the compound A1-containing SDD granules with extragranular AVICEL® PH-102, Povidone PVP-XL, and CAB-O-SIL® M-5P. The mixture was blended with magnesium stearate and compressed into tablets. The composition and characterization of 25 mg SDD tablets are summarized in Tables 2 and 3, respectively.

TABLE 1
Characterization of Granules and Final Pre-tableting Blend

| | Pre Granulated | | |
|---|---|---|---|
| Parameter | Blend | Granule | Final Blend |
| Ribbon solid franction | N/A | 0.61 | N/A |
| Bulk density (g/mL) | 0.21 | 0.40 | 0.38 |
| Tapped density after | 0.31 | 0.53 | 0.49 |
| 2000 taps (g/mL) | | | |
| Carr index | 32 | 25 | 22 |
| Apparent density by helium | 1.06 | N/A | 1.41 |
| pycnometer (g/mL) | | | |

TABLE 2
Tablet Formulation I (25 mg Compound A1-SDD)

| | | Composition | | |
|---|---|---|---|---|
| | | w % of | w % of | |
| Component | Function | Granulation | Tablet | mg/Tablet |

Intragranular Components

| | | | | |
|---|---|---|---|---|
| 25% Compound A1/PVP K30 | Active | 97.75 | 25.00^{a} | 100.0^{a} |
| SDD | | | | |
| Colloidal silicon dioxide (NF) | Glidant | 2.0 | 0.54^{a} | 2.16^{a} |
| (CAB-O-SIL ® M-5P) | | | | |
| Magnesium stearate (NF) | Lubricant | 0.25 | 0.07^{a} | 0.28^{a} |

Extragranular Components

| | | | | |
|---|---|---|---|---|
| Microcrystalline cellulose (NF) | Filler | NA | 63.64^{a} | 254.56^{a} |
| (AVICEL ® PH 102) | | | | |
| POLYPLASDONE ® (PVP-XL) | Disintegrant | NA | 10.00 | 40.00 |
| (NF) | | | | |
| Colloidal silicon dioxide (NF) | Glidant | NA | 0.50 | 2.00 |
| (CAB-O-SIL ® M-5P) | | | | |
| Magnesium stearate (NF) | Lubricant | NA | 0.25 | 1.00 |

| | | | |
|---|---|---|---|
| Total | 100% | 400 | mg |

^{a}Adjustments may be required to the granules and the filler (microcrystalline cellulose) to account for SDD potency.

TABLE 3
Characterization of Tablet Formulation I
(25 mg Compound A1-SDD)

| | | | Relative | |
|---|---|---|---|---|
| | Target | | Standard | |
| Parameter | Value | Actual Value | Deviation | Range |
| Weight (mg) | 400.0 | 399.2 (N = 3) | 0.09 | 398.8-399.5 |
| Height (mm) | NA | 6.52 (N = 3) | 0.23 | 6.51-6.54 |
| Hardness (kP) | 18.0 | 17.7 (N = 3) | 0.86 | 17.5-17.8 |
| Compression | NA | 4.5 (N = 3) | 0.00 | 4.5-4.5 |
| force (kN) | | | | |
| Solid fraction | 0.60 | 0.61 (N = 3) | 1.05 | |
| Disintegration time | <300 | 19 (N = 3) | NA | 17-23 |
| (sec) | | | | |
| Friability at | <0.2 | 0.20 (N = 2)_{—} | NA | NA |
| 100 drops (%) | | | | |

The corresponding 50 mg SDD tablets were also made and the composition and characterization are summarized in Tables 4 and 5, respectively.

TABLE 4
Tablet Formulation II (50 mg Compound A1-SDD)

| | | Composition | | |
|---|---|---|---|---|
| | | w % of | w % of | |
| Component | Function | Granulation | Tablet | mg/Tablet |

Intragranular Components

| | | | | |
|---|---|---|---|---|
| 25% Compound A1/PVP K30 | Active | 97.75 | 25.00^{a} | 200.00^{a} |
| SDD | | | | |
| Colloidal silicon dioxide (NF) | Glidant | 2.0 | 0.54^{a} | 4.32^{a} |
| (CAB-O-SIL ® M-5P) | | | | |
| Magnesium stearate (NF) | Lubricant | 0.25 | 0.07^{a} | 0.56^{a} |

Extragranular Components

| | | | | |
|---|---|---|---|---|
| Microcrystalline cellulose (NF) | Filler | NA | 63.64^{a} | 509.12^{a} |
| (AVICEL ® PH 102) | | | | |
| POLYPLASDONE ® (PVP- | Disintegrant | NA | 10.00 | 80.00 |
| XL) (NF) | | | | |
| Colloidal silicon dioxide (NF) | Glidant | NA | 0.50 | 4.00 |
| (CAB-O-SIL ® M-5P) | | | | |
| Magnesium stearate (NF) | Lubricant | NA | 0.25 | 2.00 |

| | | | |
|---|---|---|---|
| Total | 100% | 800 | mg |

^{a}Adjustments may be required to the granules and the filler (microcrystalline cellulose) to account for SDD potency.

TABLE 5
Characterization of Tablet Formulation I
(50 mg Compound A1-SDD)

| | | | Relative | |
|---|---|---|---|---|
| | Target | | Standard | |
| Parameter | Value | Actual Value | Deviation | Range |
| Weight (mg) | 800.0 | 799.3 (N = 82) | 0.84 | 774.9-815.2 |
| Height (mm) | NA | 6.28 (N = 15) | 0.15 | 6.25-6.29 |
| Hardness (kP) | 22.0 | 21.9 (N = 15) | 4.5 | 20.5-23.4 |
| Compression | NA | 12.0 (N = 3) | 1.0 | 11.8-12.0 |
| force (kN) | | | | |
| Ejection force (N) | <500 | 328 (N = 3) | 1.0 | 326-332 |
| Solid fraction | 0.60 | 0.61 (N = 3) | 1.05 | |
| Disintegration | <300 | 127 (N = 3) | 4.8 | 120-131 |
| time (sec) | | | | |
| Friability at | <0.2 | 0.04 (N = 2)_{—} | NA | NA |
| 100 drops (%) | | | | |
| Content | 100 | 102.6 (N = 10) | 2.56 | 97.5-107.0 |
| uniformity | | | | |
| (% LC) | | | | |
| USP <905) AV | NMT 15.0 | 7.4 (N = 1) | NA | NA |

Additional tablets of compound A1-containing SDD were prepared and their compositions are summarized in Tables 6 to 13.

TABLE 6
Tablet Formulation III

| | | |
|---|---|---|
| Ingredient | Composition (w %) | mg/Tablet |

Intragranular Components

| | | |
|---|---|---|
| 25% Compound A1/PVP K30 SDD | 25.00 | 200.00 |
| Colloidal silicon dioxide (NF) | 0.5 | 4.0 |
| (CAB-O-SIL ® M-5P) | | |
| Magnesium stearate (NF) | 0.1 | 0.8 |

Extragranular Components

| | | |
|---|---|---|
| Microcrystalline cellulose (NF) | 51.0 | 408.0 |
| (AVICEL ® PH 102) | | |
| POLYPLASDONE ® (PVP-XL) | 12.9 | 103.2 |
| (NF) | | |
| Crospovidone CL | 10 | 80.0 |
| Magnesium stearate (NF) | 0.5 | 4.0 |
| Total | 100.00 | 800.0 |

TABLE 7
Tablet Formulation IV

| | | |
|---|---|---|
| Ingredient | Composition (w %) | mg/Tablet |

Intragranular Components

| | | |
|---|---|---|
| 25% Compound A1/PVP K30 SDD | 25.00 | 200.00 |
| Colloidal silicon dioxide (NF) | 0.5 | 4.0 |
| (CAB-O-SIL ® M-5P) | | |
| Magnesium stearate (NF) | 0.1 | 0.8 |

Extragranular Components

| | | |
|---|---|---|
| Microcrystalline cellulose (NF) | 31.50 | 252.0 |
| (AVICEL ® PH 102) | | |
| Sodium Lauryl sulfate | 19.50 | 156.0 |
| Tartaric Acid | 12.9 | 103.2 |
| Crospovidone CL | 10.0 | 80.0 |
| Magnesium stearate (NF) | 5.0 | 4.0 |
| Total | 100.00 | 800.0 |

TABLE 8
Tablet Formulation V

| | | |
|---|---|---|
| Ingredient | Composition (w %) | mg/Tablet |

Intragranular Components

| | | |
|---|---|---|
| 25% Compound A1/PVP K30 SDD | 25.0 | 200.00 |
| Colloidal silicon dioxide (NF) | 0.5 | 4.0 |
| (CAB-O-SIL ® M-5P) | | |
| Magnesium stearate (NF) | 0.1 | 0.8 |

Extragranular Components

| | | |
|---|---|---|
| Microcrystalline cellulose (NF) | 63.9 | 511.2 |
| (AVICEL ® PH 102) | | |
| Crospovidone CL | 10.0 | 80.0 |
| Magnesium stearate (NF) | 5.0 | 4.0 |
| Total | 100.00 | 800.0 |

TABLE 9
Tablet Formulation VI

| | | |
|---|---|---|
| | Composition | |
| Ingredient | (w %) | mg/Tablet |

Intragranular Components

| | | |
|---|---|---|
| 25% Compound A1/HPMCAS-MG SDD | 25.0 | 200.00 |
| Colloidal silicon dioxide (NF) | 0.5 | 4.0 |
| (CAB-O-SIL ® M-5P) | | |
| Magnesium stearate (NF) | 0.1 | 0.8 |

Extragranular Components

| | | |
|---|---|---|
| Microcrystalline cellulose (NF) | 51.0 | 408.0 |
| (AVICEL ® PH 102) | | |
| Sodium lauryl sulfate | 12.9 | 103.2 |
| Crospovidone CL | 10.0 | 80.0 |
| Magnesium stearate (NF) | 5.0 | 4.0 |
| Total | 100.00 | 800.0 |

TABLE 10
Tablet Formulation VII

| | | |
|---|---|---|
| | Composition | |
| Ingredient | (w %) | mg/Tablet |

Intragranular Components

| | | |
|---|---|---|
| 25% Compound A1/HPMCAS-MG SDD | 25.0 | 200.00 |
| Colloidal silicon dioxide (NF) | 0.5 | 4.0 |
| (CAB-O-SIL ® M-5P) | | |
| Magnesium stearate (NF) | 0.1 | 0.8 |

Extragranular Components

| | | |
|---|---|---|
| Microcrystalline cellulose (NF) | 31.50 | 252.0 |
| (AVICEL ® PH 102) | | |
| Sodium lauryl sulfate | 19.50 | 156.0 |
| Tartaric acid | 12.9 | 103.2 |
| Crospovidone CL | 10.0 | 80.0 |
| Magnesium stearate (NF) | 5.0 | 4.0 |
| Total | 100.00 | 800.0 |

TABLE 11
Tablet Formulation VIII

| | | |
|---|---|---|
| | Composition | |
| Ingredient | (w %) | mg/Tablet |

Intragranular Components

| | | |
|---|---|---|
| 25% Compound A1/HPMCAS-MG SDD | 25.0 | 200.00 |
| Microcrystalline cellulose (NF) | 33.5 | 268 |
| (AVICEL ® PH 102) | | |
| Lactose | 33.5 | 268 |
| (FAST FLO ® 316) | | |
| Croscarmellose sodium (AC-DI-SOL ®) | 6.0 | 48 |
| Colloidal silicon dioxide (NF) | 1 | 8 |
| (CAB-O-SIL ® M-5P) | | |
| Magnesium stearate (NF) | 0.25 | 2 |

Extragranular Components

| | | |
|---|---|---|
| Colloidal silicon dioxide (NF) | 0.5 | 4 |
| (CAB-O-SIL ® M-5P) | | |
| Magnesium stearate (NF) | 0.25 | 2 |
| Total | 100.00 | 800.0 |

TABLE 12
Tablet Formulation IX

| | | |
|---|---|---|
| Ingredient | Composition (w %) | mg/Tablet |

Intragranular Components

| | | |
|---|---|---|
| 25% Compound A1/PVP K30 SDD | 25.0 | 200.00 |
| Colloidal silicon dioxide (NF) | 0.5 | 4.0 |
| (CAB-O-SIL ® M-5P) | | |
| Magnesium stearate (NF) | 0.1 | 0.8 |

Extragranular Components

| | | |
|---|---|---|
| Microcrystalline cellulose (NF) | 51.0 | 408.0 |
| (AVICEL ® PH 102) | | |
| Tartaric acid | 12.9 | 103.2 |
| Crospovidone CL | 10.0 | 80.0 |
| Magnesium stearate (NF) | 5.0 | 4.0 |
| Total | 100.00 | 800.0 |

TABLE 13
Tablet Formulation X

| Ingredient | Composition (w %) | mg/Tablet |
|---|---|---|
| 25% Compound A1/PVP K30 | 34.13 | 204.80 |
| SDD spray-dried granules | | |
| Microcrystalline cellulose (NF) | 55.36 | 332.2 |
| (AVICEL ® PH 102) | | |
| Crospovidone CL | 10.0 | 60.0 |
| Magnesium stearate (NF) | 0.5 | 3 |
| Total | 100.00 | 600.0 |

### Example 4

### Tablet Stability

The stability of tablet formulation II (50 mg SDD) packaged into foil/foil blisters as well as packaged into HDPE bottles was measured at 5° C., 25° C./60% RH, and 40° C./75% RH and the results are summarized in Tables 14 to 19.

TABLE 14
Storage Stability of 50 mg SDD Tablets in Blisters at 5° C.

| Time (Months) | 0 | 0.5 | 1 | 3 | 6 | 9 |
|---|---|---|---|---|---|---|
| Cmpd. A1 (%) | 100.1 | 104.8 | 102.0 | 101.4 | 106.8 | 107.5 |
| Appearance | White | White | White | White | White | White |
| | uncoated | uncoated | uncoated | uncoated | uncoated | uncoated |
| | tablets | tablets | tablets | tablets | tablets | tablets |
| Total Impurities | 0.6 | 0.7 | 0.6 | 0.6 | 0.5 | 0.5 |
| (% area) | | | | | | |
| Water Content | 5.22 | 5.83 | 5.67 | 5.53 | 5.48 | 5.70 |
| (wt %) | | | | | | |
| Dissolution at | 102 | 101 | 105 | 99 | 101 | 100 |
| 15 min (%) | | | | | | |
| Disintegration | 36 ± 4 | 40 ± 6 | 38 ± 4 | 37 ± 6 | 39 ± 6 | 43 ± 6 |
| (Sec) | | | | | | |
| XRPD | Amorphous | Amorphous | Amorphous | Amorphous | Amorphous | Amorphous |
| AUC_{IB} | 29,600 | 31,500 | 30,900 | 34,500 | 33,500 | 35,503 |
| (min · μg/mL) | | | | | | |

TABLE 15
Storage Stability of 50 mg SDD Tablets in Blisters at 25° C./60% RH

| Time (Months) | 0 | 0.5 | 1 | 3 | 6 | 9 |
|---|---|---|---|---|---|---|
| Cmpd. A1 (%) | 100.1 | 98.8 | 97.7 | 104.7 | 102.9 | 102.3 |
| Appearance | White | White | White | White | White | White |
| | uncoated | uncoated | uncoated | uncoated | uncoated | uncoated |
| | tablets | tablets | tablets | tablets | tablets | tablets |
| Total Impurities | 0.6 | 0.7 | 0.6 | 0.6 | 0.5 | 0.5 |
| (% area) | | | | | | |
| Water Content | 5.22 | 5.84 | 5.65 | 5.52 | 5.57 | 5.66 |
| (wt %) | | | | | | |
| Dissolution at | 102 | 99 | 99 | 98 | 100 | 100 |
| 15 min (%) | | | | | | |
| Disintegration | 36 ± 4 | 42 ± 5 | 65 ± 11 | 47 ± 14 | 43 ± 10 | 46 ± 3 |
| (Sec) | | | | | | |
| XRPD | Amorphous | Amorphous | Amorphous | Amorphous | Amorphous | Amorphous |
| AUC_{IB} | 29,600 | 34,600 | 30,300 | 27,000 | 37,000 | 36,067 |
| (min · μg/mL) | | | | | | |

TABLE 16
Storage Stability of 50 mg SDD Tablets in Blisters at 40° C./75% RH

| | Time (Months) | | | | |
|---|---|---|---|---|---|
| | 0 | 0.5 | 1 | 3 | 6 |
| Cmpd. A1 (%) | 100.1 | 105.5 | 105.4 | 102 | 102.8 |
| Appearance | White | White | White | White | White |
| | uncoated | uncoated | uncoated | uncoated | uncoated |
| | tablets | tablets | tablets | tablets | tablets |
| Total Impurities | 0.6 | 0.7 | 0.6 | 0.6 | 0.5 |
| (% area) | | | | | |
| Water Content | 5.22 | 5.82 | 5.66 | 5.41 | 5.50 |
| (wt %) | | | | | |
| Dissolution at 15 min | 102 | 101 | 102 | 102 | 103 |
| (%) | | | | | |
| Disintegration | 36 ± 4 | 59 ± 6 | 52 ± 6 | 48 ± 8 | 71 ± 27 |
| (Sec) | | | | | |
| XRPD | Amorphous | Amorphous | Amorphous | Amorphous | Amorphous |
| AUC_{IB} | 29,600 | 33,000 | 30,500 | 33,600 | 33,500 |
| (min · μg/mL) | | | | | |

TABLE 17
Storage Stability of 50 mg SDD Tablets in Blisters at 5° C.

| Time (Months) | 0 | 1 | 3 | 6 | 9 | 12 |
|---|---|---|---|---|---|---|
| Cmpd. A1 (%) | 103 | 102.1 | 98.1 | 104.3 | 97.1 | 104.8 |
| Appearance | White | Off-White | White | Off-White | Off-White | White |
| | uncoated | uncoated | uncoated | uncoated | uncoated | uncoated |
| | tablets | tablets | tablets | tablets | tablets | tablets |
| Total Impurities | 0.6 | 0.5 | 0.6 | 0.7 | 0.5 | 0.6 |
| (% area) | | | | | | |
| Water Content | 5.34 | 5.00 | 4.81 | 4.60 | 4.60 | 4.76 |
| (wt %) | | | | | | |
| Dissolution at | 94 | 100 | 102 | 101 | 101 | 98% |
| 15 min (%) | | | | | | |
| Disintegration | 32 ± 3 | 53 ± 23 | 55 ± 10 | 74 ± 14 | 71 ± 26 | 68 ± 15 |
| (Sec) | | | | | | |
| XRPD | Amorphous | Amorphous | Amorphous | Amorphous | Amorphous | Amorphous |
| AUC_{IB} | 32,520 | 33,300 | 32,000 | 39,800 | 27,500 | 26,800 |
| (min · μg/mL) | | | | | | |

TABLE 18
Storage Stability of 50 mg SDD Tablets in Bottles at 25° C./60% RH

| Time (Months) | 0 | 1 | 3 | 6 | 9 | 12 |
|---|---|---|---|---|---|---|
| Cmpd. A1 (%) | 103 | 96.8 | 102.0 | 106.1 | 105.2 | 102.6 |
| Appearance | White | Off-White | White | Off-White | Off-White | White |
| | uncoated | uncoated | uncoated | uncoated | uncoated | uncoated |
| | tablets | tablets | tablets | tablets | tablets | tablets |
| Total Impurities | 0.6 | 0.6 | 0.6 | 0.7 | 0.5 | 0.6 |
| (% area) | | | | | | |
| Water Content | 5.34 | 4.75 | 4.61 | 4.55 | 4.78 | 4.74 |
| (wt %) | | | | | | |
| Dissolution at | 94 | 103 | 104 | 99 | 101 | 93 |
| 15 min (%) | | | | | | |
| Disintegration | 32 ± 3 | 58 ± 33 | 67 ± 15 | 76 ± 11 | 91 ± 11 | 75 ± 16 |
| (Sec) | | | | | | |
| XRPD | Amorphous | Amorphous | Amorphous | Amorphous | Amorphous | Amorphous |
| AUC_{IB} | 32,520 | 36,200 | 37,800 | 31,700 | 27,400 | 25,600 |
| (min · μg/mL) | | | | | | |

TABLE 19
Storage Stability of 50 mg SDD Tablets in Bottles at 40° C./75% RH

| | Time (Months) | | | |
|---|---|---|---|---|
| | 0 | 1 | 3 | 6 |
| Cmpd. A1 (%) | 103 | 109.2 | 106.8 | 103.0 |
| Appearance | White | Off-White | White | Off-White |
| | uncoated | uncoated | uncoated | uncoated |
| | tablets | tablets | tablets | tablets |
| Total Impurities | 0.6 | 0.5 | 0.6 | 0.7 |
| (% area) | | | | |
| Water Content (wt %) | 5.34 | 4.70 | 4.96 | 5.17 |
| Dissolution at 15 min (%) | 94 | 103 | 100 | 102 |
| Disintegration (Sec) | 32 ± 3 | 49 ± 1 | 58 ± 13 | 84 ± 17 |
| XRPD | Amorphous | Amorphous | Amorphous | Amorphous |
| AUC_{IB} (min · μg/mL) | 32,520 | 37,200 | 39,900 | 36,000 |

### Example 5

### Phase I, Open-Label Study to Evaluate the Relative Bioavailability of Compound A1 in Healthy Subjects

This was a randomized, open-label, relative bioavailability study employing a crossover and parallel design. Subjects were randomized to one of four sequences, in which they received a single dose of each formulation, followed by a 6-day washout per treatment period.

In the open-label study, compound A1 (100 mg) was administered orally as a suspension or tablet under fasted conditions. Dosing occurred on the first day of each treatment period, Days 1 and 8, separated by a washout period of 7 days.

For treatment periods 1 and 2, subjects were randomized equally into one of four treatment groups and received a single dose of 100 mg of compound A1 as Suspension I and 100 mg of a compound A1 as two 50 mg tablets (Tablet Formulation II) with water (Sequences 1 and 2) or non-caffeinated regular (not sugar-free) COCA COLA® (Sequences 3 and 4) on days 1 and 8 in a crossover fashion as shown in Table 20.

TABLE 20
Treatment Sequences

| | | Period 1 | Period 2 |
|---|---|---|---|
| Sequences | Chaser | (Days 1 to 7) | (Days 8 to 14) |
| 1 (n-6) | Water | Suspension I | Tablet I |
| 2 (n-6) | Water | Tablet I | Suspension I |
| 3 (n-6) | COCA COLA ® | Suspension I | Tablet I |
| 4 (n-6) | COCA COLA ® | Tablet I | Suspension I |

Suspension I was an oral suspension formulation of compound A1-containing SDD as prepared in Example 1, comprising 20% (v/v) expresso flavoring syrup and 0.5% (w/v) methylcellulose (METHOCEL® A4M premium, the Dow Chemical Company) in water. The compound concentration in the suspension was 10 mg/mL.

Dosing occurred on the morning of each dosing day. Subjects observed a fasting period of approximately 10 hours prior to dosing on Days 1 and 8. On Days 1 and 8, a single oral dose of one of two formulations was administered as a 2×50 mg tablets (Tablet I) or 10 mL of a suspension containing 10 mg/mL compound A1 (Suspension I) according to the randomized treatment sequence assignment. Dosing on Days 1 and 8 was immediately followed by administration of 240 mL of water (Sequences 1 and 2) or non-caffeinated regular COCA COLA® (sequences 3 and 4).

Each subject underwent three PK sessions beginning at the predose time point on Days 1 and 8 and ending 144 hours after dosing in each treatment period. The duration of the study was up to 42 day.

The subjects in the study were healthy males and females between 19 and 65 years of age with a body mass index between 18 and 35 kg/m². A total of 24 subjects in the test were randomized to one of four sequences in which they received a single dose of each formulation followed by a six day washout per treatment period treatment. 100 mg of Compound A1 was administered orally as suspension or a tablet under fasted conditions. Dosing occurred on the first day of each treatment period, days 1, and 8, separated by a washout period of 7 days.

PK parameters were calculated from the plasma concentration vs. time data of compound A1 on Days 1 to 7 (144 hrs) and Days 8 to 14 (144 hrs) using WinNonlin Version 5.2 and SAS® Version 9.1.3. For the calculations of the PK parameters, plasma concentrations below the limit of quantitation were treated as zero before the first quantifiable concentration and as missing thereafter. Linear regression was performed using at least 3 data points. The lower limit of quantitation (LLOQ) was 0.100 ng/mL for the analysis of the plasma concentration of compound A1.

The pharmacokinetic parameters of SDD Tablet of Formulation I are summarized in Table 21.

TABLE 21
Pharmacokinetic Parameters of Compound A1

| PK | Water as a Chaser | | COCA COLA ® as a Chaser | |
|---|---|---|---|---|
| parameters | Suspension I | Tablet I | Suspension I | Tablet I |
| Cₘₐₓ (ng/mL) | 57 ± 24 | 38 ± 14 | 71 ± 26 | 58 ± 28 |
| Tₘₐₓ (hrs)^{a} | 4.0 (4.0, 4.0) | 4 (3.0, 6.0) | 4.0 (4.0, 6.0) | 4.0 (3.0, 6.0) |
| AUC₀₋ₜ (ng · hr/mL) | 1054 ± 375 | 815 ± 310 | 1362 ± 622 | 1089 ± 627 |
| AUC₀₋₁₂ₕ (ng · hr/mL) | 458 ± 187 | 325 ± 116 | 558 ± 194 | 463 ± 229 |
| AUC₀₋₂₄ₕ (ng · hr/mL) | 675 ± 259 | 498 ± 180 | 848 ± 300 | 693 ± 346 |
| AUC_{0 inf} (ng · hr/mL) | 1064 ± 378 | 823 ± 315 | 1381 ± 647 | 1103 ± 644 |
| T_{1/2z} (hrs) | 22 ± 3 | 21 ± 3 | 21 ± 6 | 22 ± 7 |
| Lambda z (1/hr) | 0.032 ± 0.004 | 0.033 ± 0.005 | 0.035 ± 0.009 | 0.033 ± 0.010 |
| CL/F (L/hr) | 110 ± 53 | 135 ± 42 | 85 ± 32 | 160 ± 212 |
| Vz/F (L) | 3472 ± 1656 | 4074 ± 1314 | 2403 ± 672 | 6628 ± 12901 |

^{a}Tₘₐₓ is presented as Median (Minimum, Maximum).

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1-89. (canceled)

90. Spray-dried particles comprising a compound of [(S)-1-((S)-2-{6-[6-(4-{(S)-2-[1-((R)-2-methoxycarbonylamino-2-phenylacetyl)-pyrrolidin-2-yl]-3H-imidazol-4-yl}-phenyl)-thieno[3,2-b]thiophen-3-yl]-1H-benzoimidazol-2-yl}-pyrrolidine-1-carbonyl)-2-methylpropyl]-carbamic acid methyl ester, having the structure of Formula A1; 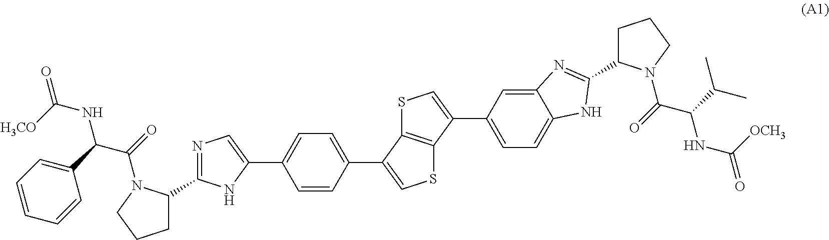
or [(S)-1-((S)-2-{5-[4-(6-{(S)-2-[1-((R)-2-methoxycarbonylamino-2-phenylacetyl)-pyrrolidin-2-yl]-3H-benzoimidazol-5-yl}-thieno[3,2-b]-thiophen-3-yl)-phenyl]-1H-imidazol-2-yl}pyrrolidine-1-carbonyl)-2-methylpropyl]-carbamic acid methyl ester, having the structure of Formula A2; 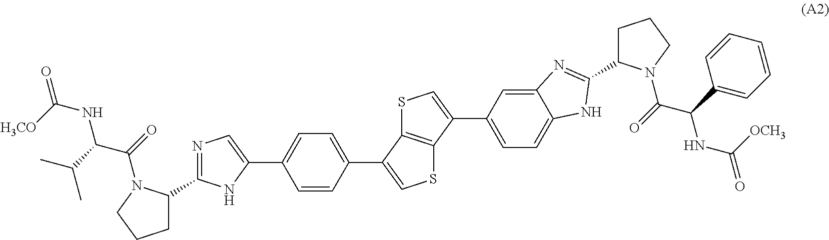
or an isotopic variant thereof, or a pharmaceutically acceptable salt thereof, or a pharmaceutically acceptable solvate thereof; and a first pharmaceutically acceptable excipient.

91. The spray-dried particles of claim 90, wherein the compound is an amorphous solid.

92. The spray-dried particles of claim 90, having:
(i) an average particle size ranging from about 0.1 to about 500 jam;
(ii) a bulk density ranging from about 0.01 to about 1 g/mL;
(iii) a tapped density ranging from about 0.01 to about 1 g/mL;
(iv) a residual water content of no greater than about 30% by weight;
(v) a residual organic solvent content of no greater than about 5,000 ppm;
(vi) a residual methanol content of no greater than about 3,000 ppm;
(vii) a residual tetrahydrofuran content of no greater than about 750 ppm; or
(viii) a stability at a 25° C. and 60% relative humidity for a period from about 6 months to about 10 years.

93. The spray-dried particles of claim 90, wherein the first pharmaceutically acceptable excipient is a hypromellose, a hypromellose acetate succinate, a hypromellose phthalate, a methacrylic acid and ethyl acrylate copolymer, a poloxamer, a polyethylene glycol, a povidone, a tocopherol polyethylene glycol succinate, or a mixture thereof.

94. The spray-dried particles of claim 90, having:
(i) an average particle size ranging from about 1 to about 50 m;
(ii) a bulk density ranging from about 0.05 to about 0.3 g/mL;
(iii) a tapped density ranging from about 0.2 to about 0.6 g/mL;
(iv) a residual water content ranging from about 1% to about 20% by weight;
(v) a residual organic solvent content of no greater than about 0.05% by weight,
(vi) a residual methanol content of no greater than about 3,000 ppm;
(vii) a residual THF content of no greater than about 750 ppm; or
(viii) a stability at a 25° C. and 60% relative humidity for a period from about 6 months to about 10 years.

95. The spray-dried particles of claim 94, wherein the first pharmaceutically acceptable excipient is:
(i) hypromellose 2910 substitution type;
(ii) a hypromellose acetate succinate having an average molecular weight of about 18,000 Da;
(iii) a hypromellose phthalate having an average molecular weight of about 84,000 Da;
(iv) a methacrylic acid and ethyl acrylate copolymer having an average molecular weight of about 320,000 Da;
(v) a poloxamer having an average molecular weight of about 1,800 Da;
(vi) a poloxamer having an average molecular weight of about 4,000 Da;
(vii) a polyvinyl pyrrolidone having an average molecular weight from about 40,000 Da to about 60,000 Da; or
(viii) a polyethylene glycol having an average molecular weight of about 8,000 Da; or
(ix) a mixture of a tocopherol polyethylene glycol succinate and a poloxamer having an average molecular weight of about 1,800 Da.

96. A pharmaceutical composition comprising the spray-dried particles of claim 90 and a second pharmaceutically acceptable excipient, wherein the second pharmaceutically acceptable excipient is a disintegrant, a filler, a glidant, a lubricant, an organic acid, a surfactant, or a mixture thereof.

97. The pharmaceutical composition of claim 96, comprising (i) from about 10 to about 50% of the spray-dried particles; and (ii) from about 0.5 to about 25% by weight of a disintegrant, from about 25 to about 80% by weight of a filler, from about 0.1 to about 5% by weight of a glidant, and from about 0.05 to about 2% by weight of a lubricant.

98. The pharmaceutical composition of claim 97, wherein the spray-dried particles comprise the compound of Formula A1.

99. A method for treating or preventing a Flaviviridae infection or one or more symptoms thereof in a subject, comprising administering to the subject the spray-dried particles of claim 90.

100. The method of claim 99, wherein the Flaviviridae is a hepatitis C virus.

101. The method of claim 110, wherein the method comprises administering to the subject a second antiviral agent, in combination or alternation.

102. The method of claim 101, wherein the second antiviral agent is an interferon, ribavirin, amantadine, an interleukin, a NS3 protease inhibitor, a cysteine protease inhibitor, a phenathrenequinone, a thiazolidine, a benzanilide, a helicase inhibitor, a polymerase inhibitor, a nucleotide analogue, a liotoxin, acerulenin, an antisense phosphorothioate oligodeoxynucleotide, an inhibitor of IRES-dependent translation, a ribozyme, or a mixture thereof.

103. The method of claim 102, wherein the subject is a human.
